SEQUENCE LISTING (96 sequences)

SEQ ID NO: 1 - DNA, 600 bp, synthetic construct
  misc_feature 1..600: note = Description of Artificial Sequence: Synthetic polynucleotide
  misc_feature 1..600: note = Codon optimized
  source 1..600: mol_type = other DNA
aggcccaagcaccccatcaagcatcaggggttgccacaggaagtcctcaatgaaaatctgctgaggttcttcgtggctcctttcccagaagttttcggaaaggaaaaagttaacgagctcagcaaagacatcggctctgaatccaccgaagaccaagcaatggaggacattaagcaaatggaagctgagagtatatcctcatccgaagaaatcgtcccaaacagcgtagaacaaaagcatattcagaaagaagatgttcctagtgaaagatacctcgggtatttggagcaacttctgagactgaaaaagtacaaagtgccccagctcgagatcgttccaaactccgccgaagaacgtctgcatagtatgaaggaggggatacatgcacaacagaaggaacccatgatcggagttaaccaggaactcgcttacttctaccctgaactcttcaggcagttttatcagcttgacgcttatccctccggtgcttggtactatgtaccacttggaacacaatacacagacgcaccatcattttctgacatacccaaccctatcgggtctgagaacagtgaaaaaacaacaatgcctctgtggtaa

SEQ ID NO: 2 - DNA, 510 bp, synthetic construct
  misc_feature 1..510: note = Description of Artificial Sequence: Synthetic polynucleotide
  misc_feature 1..510: note = Codon optimized
  source 1..510: mol_type = other DNA
caggagcagaaccaagaacaacctatcaggtgcgaaaaggatgagaggttcttttccgataaaattgcaaagtacattcctattcaatatgtactgtctcgctaccccagttatggacttaactactaccaacagaaacccgttgcccttataaacaatcagttcctcccttatccttactatgcaaagcctgctgccgtgcgtagtcccgcacagattctccagtggcaggttctcagcaatactgttcccgcaaaaagctgtcaggctcaacctactactatggcacgtcatcctcacccccatttgagctttatggccatccctccaaagaagaaccaagataagactgaaatcccaactataaacacaatcgcatccggggagcctacctctactcccactattgaggctgtcgaatctactgttgcaactttggaagctagtcccgaagtcaccgagagcccccctgagatcaacaccgtacaggttacatctaccgctgtatga

SEQ ID NO: 3 - DNA, 630 bp, synthetic construct
  misc_feature 1..630: note = Description of Artificial Sequence: Synthetic polynucleotide
  misc_feature 1..630: note = Codon optimized
  source 1..630: mol_type = other DNA
agggagctggaagagctgaacgtccctggcgaaatagtagagtccctcagctcatcagaagagtccattactcgtattaacaaaaagattgaaaagtttcagtcagaagagcaacaacagactgaggacgaactccaagacaagattcaccctttcgcacagacacagagtctggtctacccatttcctggtcctattcccaattcccttccccagaatatacctcccctcacccagactcctgtggtggtccccccattcctccaaccagaggttatgggtgtttctaaagtcaaagaagcaatggcccctaagcacaaagagatgccattccccaagtatcccgttgagccctttaccgagtctcagagccttacactgaccgacgtagaaaatctccatctcccactcccattgttgcaatcttggatgcaccagccccatcagcctttgccccctactgtcatgtttcccccccagagtgttctgtccttgagccaaagcaaagtgctccctgtgccccagaaagccgtaccttatccccaaagagacatgccaatacaggcctttttgctctaccaggagcctgttctcggtcccgtaagaggccctttccctatcatcgtgtag

SEQ ID NO: 4 - DNA, 624 bp, synthetic construct
  misc_feature 1..624: note = Description of Artificial Sequence: Synthetic polynucleotide
  misc_feature 1..624: note = Codon optimized
  source 1..624: mol_type = other DNA
aaaaacactatggagcacgtaagctcatccgaggagagtataatctcccaggaaacatataaacaagaaaaaaatatggcaattaacccatccaaggaaaacctgtgttccaccttttgtaaggaagtcgtgcgtaatgctaatgaagaagagtattcaatcggctccagttcagaggagtctgcagaagtagccacagaggaggttaagattactgttgatgataaacactaccaaaaggcccttaacgagataaatcagttctatcaaaaatttcctcaatatttgcaatacttgtatcagggacctattgttctcaatccttgggatcaggttaagcgtaacgccgtaccaattacaccaactctcaacagggagcagctcagcacctccgaggaaaactccaaaaagacagtggatatggagtcaactgaggtcttcacaaaaaagacaaagctcaccgaggaagagaagaacagactcaactttttgaaaaaaatatcacaaagataccaaaagtttgcactgccccaatatctcaagactgtttaccaacaccaaaaggctatgaagccctggattcaaccaaagaccaaagtcataccctacgtgaggtatttgtaa

SEQ ID NO: 5 - DNA, 612 bp, synthetic construct
  misc_feature 1..612: note = Description of Artificial Sequence: Synthetic polynucleotide
  misc_feature 1..612: note = Codon optimized
  source 1..612: mol_type = other DNA
cgtccgaaacatccaatcaagcaccaaggcctcccacaggaggtcctcaacgaaaaccttttacgtttttttgtggcacccttccctgaggtcttcggaaaggaaaaagtgaatgaactttcaaaggacattggcagcgaatccacggaagaccaggcgatggaggatattaagcagatggaggctgaaagtattagctcttccgaggagatagttcctaattccgtggaacaaaagcacattcaaaaagaggatgtcccgagtgagagatacctgggctatctcgaacagcttctgagactaaaaaagtataaggtcccgcaactggaaattgttccaaatagcgccgaagaaaggttacattccatgaaagaaggcattcatgctcagcaaaaggaacctatgatcggagtaaaccaggaacttgcctatttttacccggagttgttccgtcagttctatcagttggatgcatacccatcaggggcatggtactatgtacctctcggtacccaatacacggacgctccttctttctccgatatacccaatcccataggtagcgaaaactctgagaaaacaactatgcccctctggcatgacgaactttag

SEQ ID NO: 6 - DNA, 522 bp, synthetic construct
  misc_feature 1..522: note = Description of Artificial Sequence: Synthetic polynucleotide
  misc_feature 1..522: note = Codon optimized
  source 1..522: mol_type = other DNA
caagaacagaatcaggagcaacccattaggtgtgagaaggacgaaaggtttttttcagacaaaatcgcgaaatacatacctattcagtacgttctcagcagataccctagttatggacttaactactaccagcaaaagcctgtggcattgataaataaccagttccttccgtacccgtactatgcgaaaccggcagcggtacgaagcccagcccagattttgcaatggcaggtattgagtaacaccgtcccggcgaaaagttgtcaagcgcaaccgaccacaatggcccgacacccgcatccacatctcagcttcatggcaatcccacccaagaaaaaccaagataaaactgaaataccgacaataaacactatagcttcaggcgagccaactagcacacccactattgaagcggtagagagtacggtcgcaaccctagaggcaagcccggaagtgactgaatctccgccggaaattaacaccgtccaggtaacctcaacagcggttcatgacgaactctag

SEQ ID NO: 7 - DNA, 639 bp, synthetic construct
  misc_feature 1..639: note = Description of Artificial Sequence: Synthetic polynucleotide
  misc_feature 1..639: note = Codon optimized
  source 1..639: mol_type = other DNA
cgtgaactagaagagcttaatgtgcctggtgagatagtcgaaagtttgtccagctcagaagaatcaattacacgtatcaacaaaaaaatagaaaagtttcaatctgaggaacaacaacagacagaggacgaattacaagataaaatacacccatttgctcagacgcaaagcttagtctatccattcccaggaccaattccgaatagcttacctcaaaacatcccgccgctcacgcagacccctgtagtcgtgccgccgtttttacaacccgaggtcatgggcgtcagcaaggtaaaagaggcaatggctcctaagcataaggagatgcctttccctaaatatcccgtcgagcctttcaccgagagccaatctttaaccttaacggacgtagagaacctacatcttcctctaccactgttacaatcctggatgcatcagccgcaccaacctcttccccctacagtaatgttccccccgcagtccgtcctatctctctctcaatccaaggtcctaccagttcctcagaaggctgtcccctaccctcagcgagacatgccgatccaggctttcttgctataccaagagccggtactaggccctgtccgagggccgtttccgataattgtccacgatgaactt

SEQ ID NO: 8 - DNA, 636 bp, synthetic construct
  misc_feature 1..636: note = Description of Artificial Sequence: Synthetic polynucleotide
  misc_feature 1..636: note = Codon optimized
  source 1..636: mol_type = other DNA
aaaaacactatggagcatgtgagcagctctgaggagtccattatatcccaggaaacatataaacaagaaaagaacatggctattaacccgtcaaaggagaatctgtgttccacgttctgtaaggaagtggtgaggaacgctaatgaggaagagtactcaatcggcagttcatccgaagaatctgctgaggtggcgacagaagaagttaagattactgtcgacgacaaacactaccagaaggccctaaatgagataaatcaattttatcagaagttcccgcagtatttgcaatatctatatcaggggccaatcgttctcaatccatgggatcaggtgaaaagaaacgcagtacctatcacacctacgctgaacagggaacaactgagcacctctgaggaaaactcaaaaaagaccgtagacatggagtccactgaggtctttaccaagaaaacaaagctaacagaggaggagaaaaatcgactgaattttctgaagaaaatcagccagcgttatcaaaagttcgctctccctcagtacctaaagacggtatatcaacaccaaaaagctatgaagccgtggatacagcccaagacgaaagtgattccatacgtgcgttatcttcacgatgaattatag

SEQ ID NO: 9 - DNA, 675 bp, synthetic construct
  misc_feature 1..675: note = Description of Artificial Sequence: Synthetic polynucleotide
  misc_feature 1..675: note = Codon optimized
  source 1..675: mol_type = other DNA
atgggttactctaagacactggttgctggcctgtttgccacacttctgctggctcccgtggtgttggccactgatcccgatccacttcaagacttctgcgtagctgacctcgatggtaaagctgtaagtgtcaacggccatccttgcaagccaatgtcaaaagcaggggacgatttcttgttttcctctaaactggctaaagctggtaacacttccacccctaatggtagtgctgtgacagagcttgacgttgcagaatggcccggaactaacactttgggagtcagtatgaaccgtgtcgattttgcccctggggggacaaatccacctcatattcacccacgtgccacagaaattggcatagtcatgaagggcgaacttctcgtgggcatccttggcagcttggactcaggcaataaactttattcccgcgtagtcagggccggtgagactttcttgattcccaggggactgatgcacctgcagttcaacgtaggaaaaactgaagccagtatggtagtctccttcaattctcaaaatcccggtattgtgttcgtccccctcactctgttctcctctaaccccccaatcccaactcccgtacttaccaaggctcttagagtcgaggctggagttgtagagctgttgaagtcaaagtttgcagccggattttaa

SEQ ID NO: 10 - DNA, 176 bp, synthetic construct
  misc_feature 1..176: note = Description of Artificial Sequence: Synthetic polynucleotide
  misc_feature 1..176: note = This sequence has elements from Arabidopsis thaliana and Glycine max.
  source 1..176: mol_type = other DNA
agagccagagagcagcctcagcaaaacaggtcgtgatatgattcaattagcttccgactcattcatccaaataccgagtcgccaaaattcaaactagactcgttaaatgaatgaatgatgcggtagacaaattggatcattgattctctttgatttgctgaggctgctctctggct

SEQ ID NO: 11 - DNA, 1644 bp, Glycine max
  source 1..1644: mol_type = other DNA
atggcgtcaattactttttctcttcttcaatttcatcgtgctcctattcttctgctaattctgctcgcgggtttcggtcactgccatattccgtcaaccctcgaaggtccctttgatcccgtcaccgttccgttcgaccccgccttgcgcggcgtcgccgtcgacttgccggaaaccgatcctcgagttcgccgccgcgtccggggtttcgagcccgaacagatttcggtttctctctctacctcccatgactccgtttggatatcttgggttacaggggagttccaaataggtctcgacatcaagcctttagaccctaaaactgtatcaagtgttgttcaatatggaacttcaagatttgaattagtgcatgaagctagaggccagtctctcatctacaaccagctctatccttttgaaggccttcagaattacacatctggaatcatccatcacgttcaactcaaaggattggaaccaagcacactatactattatcaatgtggagatccttcattgcaagccatgagtgatatatactatttcaggaccatgccaatttctggttcaaagagctacccaggcaaagtagctgtagtaggagatcttggtcttacttataatacaactactaccatcggtcacctgactagtaatgaacctgatcttcttctattgattggtgatgtaacctacgcgaatctgtacctcacaaatggaactggctctgattgttatagttgctcgtttccactaactcctatacatgaaacctaccagcctcgatgggattattggggaaggtttatgcagaatctagtttctaacgttccaataatggtggtagaaggaaatcatgaaatagaaaaacaggctgaaaacaggacatttgtggcctacagttctaggtttgcattcccctctcaagaaagtggatcttcatctacattctactattctttcaatgctggaggcattcattttattatgcttggggcttatattaactatgataaaacggctgaacaatacaagtggttggagagagatctggaaaatgttgatagatcaataactccctggcttgtagttacttggcatccaccatggtatagttcttatgaagcccattacagagaagcagagtgcatgagggtggagatggaggacctattatacgcatatggtgtggatataatatttaatggacatgttcatgcctatgagaggtcaaaccgagtttacaattacaatttagatccatgtggtcctgtatatattacagttggggatgggggcaacagagagaagatggcaatcaaattcgcagacgagcctggtcattgtcccgatccattaagtactcctgatccttatatgggtggcttttgtgcaacaaattttacgtttggtacaaaagtgagtaagttttgttgggatcgccagccagattacagtgctttcagagaaagtagctttggctatgggattctagaggtgaaaaatgaaacttgggctttgtggagttggtatcgtaatcaggactcttacaaggaagttggggatcaaatttacatagtgagacaacctgatatatgccccatccatcaaagggtgaacatagattgcattgcttcgatataa

SEQ ID NO: 12 - DNA, 1284 bp, synthetic construct
  misc_feature 1..1284: note = Description of Artificial Sequence: Synthetic polynucleotide
  misc_feature 1..1284: note = Codon optimized
  source 1..1284: mol_type = other DNA
atggtcccacttgcaagtaccaccaccggcaccgggaccgctaccggtacatcaactgcagccgaaccttccgccactgtgccatttgcaagtacagatccaaaccccgtcctctggaatgaaacatcagatccagcacttgtgaagcctgagcgcaatcagctcggggctactatccagggtcctgataatctgcctatcgatttgcagaacccagatctgttggctcctcctaccactgaccatggttttgtagggaatgcaaagtggccattctcatttagcaaacaaagacttcaaaccggcgggtgggcaagacagcaaaatgaagtcgttcttcctctcgctaccaatctcgcttgtactaatatgaggctggaagccggggcaattagagagctccattggcataaaaacgccgaatgggcttacgtgttgaaaggtagtacccagatttccgctgtggacaatgagggaaggaactacatcagtactgtcggtccaggcgatctgtggtatttcccccctggaatcccccactcccttcaagccaccgccgacgaccctgaggggtccgagttcattttggtgtttgatagtggagccttcaatgatgacggaacctttcttctcaccgactggctgagtcacgtccctatggaagtcattctgaaaaattttagggccaagaaccccgctgcttggtcccatatacccgcccagcagttgtatatttttcccagtgagcctcccgccgataaccagccagaccccgtcagtccccagggaaccgtccccctcccatattcctttaatttctcaagtgtggaacctacccagtactcagggggtaccgccaaaattgccgatagtacaacttttaatattagtgtcgcaatcgcagtggcagaagtaacagttgagcctggagcactcagagaacttcattggcaccccaccgaagatgagtggacattcttcatctcaggcaacgcacgcgtgactattttcgcagcacaaagtgtagccagtacttttgattaccagggcggagatatagcatatgttcccgccagtatgggccactacgtcgagaacatagggaatactaccttgacctaccttgaagtgttcaacacagacagattcgccgacgtgagtcttagccagtggctggccctcacacctccatccgttgttcaagcacacctgaacctcgatgacgaaactctggccgaacttaagcagttcgccaccaaggctacagtagtgggccccgtgaat

SEQ ID NO: 13 - DNA, 1024 bp, Glycine max
  source 1..1024: mol_type = other DNA
aaagaagtaaatcatgagcttttccaactcaacatctattttttttctctcaacctttttcacatcttaagtagtctcaccctttatatatataacttatttcttaccttttacattatgtaacttttatcaccaaaaccaacaactttaaaattttattaaatagactccacaagtaacttgacactcttacattcatcgacattaacttttatctgttttataaatattattgtgatataatttaatcaaaataaccacaaactttcataaaaggttcttattaagcatggcatttaataagcaaaaacaactcaatcactttcatataggaggtagcctaagtacgtactcaaaatgccaacaaataaaaaaaaagttgctttaataatgccaaaacaaattaataaaacacttacaacaccggattttttttaattaaaatgtgccatttaggataaatagttaatatttttaataattatttaaaaagccgtatctactaaaatgatttttatttggttgaaaatattaatatgtttaaatcaacacaatctatcaaaattaaactaaaaaaaaaataagtgtacgtggttaacattagtacagtaatataagaggaaaatgagaaattaagaaattgaaagcgagtctaatttttaaattatgaacctgcatatataaaaggaaagaaagaatccaggaagaaaagaaatgaaaccatgcatggtcccctcgtcatcacgagtttctgccatttgcaatagaaacactgaaacacctttctctttgtcacttaattgagatgccgaagccacctcacaccatgaacttcatgaggtgtagcacccaaggcttccatagccatgcatactgaagaatgtctcaagctcagcaccctacttctgtgacgtgtccctcattcaccttcctctcttccctataaataaccacgcctcaggttctccgcttcacaactcaaacattctctccattggtccttaaacactcatcagtcatcacc

SEQ ID NO: 14 - DNA, 1055 bp, Glycine max
  source 1..1055: mol_type = other DNA
tttacaataaatactcaatttatctttcacaatcaaaagattgagatgttgtaagatctccgataatatacttatatcttttcatttattacgttttcaaatttgaattttaatgtgtgttgtaagtataaatttaaaataaaaataaaaacaattattatatcaaaatggcaaaaacatttaatacgtattatttaagaaaaaaatatgtaataatatatttatattttaatatctattcttatgtattttttaaaaatctattatatattgatcaactaaaatatttttatatctacacttattttgcatttttatcaattttcttgcgttttttggcatatttaataatgactattctttaataatcaatcattattcttacatggtacatattgttggaaccatatgaagtgtccattgcatttgactatgtggatagtgttttgatccaggcctccatttgccgcttattaattaatttggtaacagtccgtactaatcagttacttatccttcctccatcataattaatcttggtagtctcgaatgccacaacactgactagtctcttggatcataagaaaaagccaaggaacaaaagaagacaaaacacaatgagagtatcctttgcatagcaatgtctaagttcataaaattcaaacaaaaacgcaatcacacacagtggacatcacttatccactagctgatcaggatcgccgcgtcaagaaaaaaaaactggaccccaaaagccatgcacaacaacacgtactcacaaaggtgtcaatcgagcagcccaaaacattcaccaactcaacccatcatgagcccacacatttgttgtttctaacccaacctcaaactcgtattctcttccgccacctcatttttgtttatttcaacacccgtcaaactgcatgccaccccgtggccaaatgtccatgcatgttaacaagacctatgactataaatatctgcaatctcggcccaggttttcatcatcaagaaccagttcaatatcctagtacaccgtattaaagaatttaagatatact

SEQ ID NO: 15 - DNA, 1045 bp, Glycine max
  source 1..1045: mol_type = other DNA
ccttctcatcctctctgaatattttgagtgctcttcctagttatctagtaatgcatgaaattaaacttactaaatgtttcttcaatttaaagaaataattgtttatctgtttcaatttttttaagagaattttaaaaagataattgtttcggggagagagatataaaaaagaaaagggagaaatattaaaatgtactaaataatatgataagaaaagagagaaaaataaaagagaaaatttgtatatagttataattattcatgtaataaggattcatctctcaactgaaaatatacttaatgcagaagaaaaaatcattatttacaaacgttgagtcttgagtgggaaaagaggaggcgccgttactatacaatataagatcatagtactgacaaaatgcacagtaaaacagttcaaattgagaaggattcttaacacaccatagtatttaatatatatctttacagagacaattatgctggaggattcaggcaaagattatatattgtggatttgttttttaataattaacgcatcatatgaaagatcgatgatatatactaatggttataagaaaaatatttaacagtttctataacctttttcttttatcttttactgtaatattatttattttatttcacatttttaatcagcttatctcatttataaacgaaattgtataaaaatatacatgatgaactgaatagaacaatattgatctgatattctcatattgtataagaggatagactttgagacgcggagaatctgtaggaggggaccattcagagtgcctccaattttggtgttgttcattgtaccattgcaaatataaacgaagcatgcatgcttatgtatgaggtgtaacaaaattggaaacaatagccatgcaaggtgaagaatgtcacaaactcagcaacccttattcattgacgtgtccctcagtcactctcctctcatacctataaatcaccactcctcatgttctttccaattcaccaactccttcaaacttaattattaacacttccttagttcaat

SEQ ID NO: 16 - DNA, 1186 bp, Glycine max
  source 1..1186: mol_type = other DNA
gtctatttgcatgttcttctgcatggtattaagaagttcttagagaattaatctaagtacattttttttggtctggatcagacatcatatggatgctttcaaattcatgcgttggagattaattttactcataataggtaattatattaattaaaagaaattttacataaaaatacaacataaattattccattaaatatattattccctgtgactacaatgagataatctaagtgtatttgaaagtggaacagtagaaattataaaaattgcaatgagttgaataaaaaaggttggattaagaaagtaatctaagtacatttggaagtggaatagtagaaataaaattaaatgagttgaaattgaaaataattaaaaaaagtagggctaagaaatttctccttcaacttcatgatagcaaatattccattaggccatttgtagtttatgaatgagtatatataatcatgattttaggaattcgatctgctcgacacaaccgtgttacactttttttaaaatgtcatcataaaaataaaaaataaaagacatgttataattaagaataaggtgatcagtataaaaataagtaattttgggaaatattaaagttcaaaaaagaactattgaaagaaagaatattattatttaaaaagagaaaagaaaatgatgaaatgctattttcagttaaagaaaataagaaaaaaaaatacaaagaataattcaatgctggggctgtatatatgtttaagatgataattaatttttttttaaaaaaaagataagaattaaatattttctcctttaatttctgaatcacggttttggttctgataagacactgattagtcacccatcaaatataatgaactaattctcctattctatttcaaaattttgattatacttagattaattttctaatatacttggacctgtttttcatgcagaagatgcagatatagctagacagcacctagtaatcgtggaaccaacaccaatgtccatatcatgcatgtgtgccacctttcaaatgtaatccagtagtaaaaaaagccatgacatgtaactccacgacagagtaaaactctcagaagtacctctcgtttcatatctgcaaatcctctaatataaataactcacttcacgggttcttttctcttcacagcaaaaacaattaataaag

SEQ ID NO: 17 - DNA, 179 bp, Glycine max
  source 1..179: mol_type = unassigned DNA
ctacgaagggcgttgcggttgagggtgcagcgagagagggcaacaccggcacactggaatggcttgttgttagggttccatgtctcaatgagccctccttctgactctatacggttatccggtttgagggcattgagtttttggatctggcactcgttttgctgaggctgctctctgga

SEQ ID NO: 18 - DNA, 1000 bp, Glycine max
  source 1..1000: mol_type = other DNA
aaaatagtgtttgattttttgacacattattaagtgttttatttttaagtttaaaagcattggtatcctttcataaaaggaggtaatcttatttaagtcaaggagaattattatgggaaataaaacctttttttttaaagtgtttaatataattatatactcaaaattcgatttatgattaaatctaagtgacatttaaaaaaaattagtgtgaaaataatttatatataattttgaaaaatttatcattaatttttttttataaataaatgttaatttattagtttttattataaatgtgaatagaatggattcgaagcagcaatttctctctttctccttttccatgccaaccttatatatggtgacgaactgcatatacagtaaaacagttcaaattgagaaagattttaaacatcatagtatttgatatatatcttttacagagacaattatgctgcaggagttagataagattattgtggatgtcattttcttttttaatatttaacgcattatataaaagatgatatagtatggttataaaaaaattatttaacagtttataaaacctttttttttatcttttacagtaatattatttattttatttcacatttttttcatatccttatctcatttataaaggaaattaattgtataaaaaaaatatgatgcactgaatagaatgctgatcttattgtataaggaggatagaatttgagacgcggagaatctgtagagggggaccattcagggtgcctgcaattttggtgttgttcatgtacggttgcagatataaacgaagcatagcttatgtatgaggtgtaacaaaattggaaacaatagccatgcaaggtgaagaatgtcaccaactcagaaacccttcttcattgacgtgtccctcactcactctcctctcttcactataaatcgccactcttcgtgttctccacttcaccaactccttcaaacttattaacactttccttagttcaat

SEQ ID NO: 19 - DNA, 146 bp, Glycine max
  source 1..146: mol_type = other DNA
tttctgtctcatttggcattgcgtattgggaaaagcagaaccccagtcacaacaagtgcctccgaagttgcaatagcgagaaagactcctacaggaaccaagcatgccacgctcgttgcaacctccttaaggtggaggaagaagaa

SEQ ID NO: 20 - DNA, 150 bp, Glycine max
  source 1..150: mol_type = other DNA
ttcttcttcctccaccttaaggaggttgcaacgagcgtggcatgcttggttcctgtaggagtctttctcgctattgcaacttcggaggcacttgttgtgactggggttctgcttttcccaatacgcaatgccaaatgagacagaaactga

SEQ ID NO: 21 - DNA, 114 bp, Glycine max
  source 1..114: mol_type = other DNA
atcgagaattttaaggttgagtgtcctaatgtgaagtacaccgagactgagattcagtccgtgtacaactacgaaaccaccgaacttgttcacgagaacaggaatggcacctat

SEQ ID NO: 22 - DNA, 114 bp, Glycine max
  source 1..114: mol_type = other DNA
ataggtgccattcctgttctcgtgaacaagttcggtggtttcgtagttgtacacggactgaatctcagtctcggtgtacttcacattaggacactcaaccttaaaattctcgat

SEQ ID NO: 23 - DNA, 1081 bp, Glycine max
  source 1..1081: mol_type = other DNA
acaaaattaagaactgatacatcttgttttttgtcactgaagataaacacgtgatctttggcaaaacataaaggccaacaaaacaaacttgtctcatccctgaatgattcgaatgccatcgtatgcgtgtcacaaagtggaatacagcaatgaacaaatgctatcctcttgagaaaagtgaatgcagcagcagcagcagactagagtgctacaaatgcttatcctcttgagaaaagtgaatgcagcggcagcagacctgagtgctatatacaattagacacagggtctattaattgaaattgtcttattattaaatatttcgttttatattaattttttaaattttaattaaatttatatatattatatttaagacagatatatttatttgtgattataaatgtgtcactttttcttttagtccatgtattcttctattttttcaatttaactttttatttttatttttaagtcactcttgatcaagaaaacattgttgacataaaactattaacataaaattatgttaacatgtgataacatcatattttactaatataacgtcgcattttaacgtttttttaacaaatatcgactgtaagagtaaaaatgaaatgtttgaaaaggttaattgcatactaactattttttttcctataagtaatcttttttgggatcaattgtatatcattgagatacgatattaaatatgggtaccttttcacaaaacctaacccttgttagtcaaaccacacataagagaggatggatttaaaccagtcagcaccgtaagtatatagtgaagaaggctgataacacactctattattgttagtacgtacgtatttccttttttgtttagtttttgaatttaattaattaaaatatatatgctaacaacattaaattttaaatttacgtctaattatatattgtgatgtataataaattgtcaacctttaaaaattataaaagaaatattaattttgataaacaacttttgaaaagtacccaataatgctagtataaataggggcatgactccccatgcatcacagtgcaatttagctgaagcaaagca

SEQ ID NO: 24 - DNA, 179 bp, Glycine max
  source 1..179: mol_type = other DNA
tccagagagcagcctcagcaaaacgagtgccagatccaaaaactcaatgccctcaaaccggataaccgtatagagtcagaaggagggctcattgagacatggaaccctaacaacaagccattccagtgtgccggtgttgccctctctcgctgcaccctcaaccgcaacgcccttcgtag

SEQ ID NO: 25 - DNA, 211 bp, Solanum tuberosum
  source 1..211: mol_type = other DNA
ccaattggtaagtttctgcttctacctttgatatatatataataattatcattaattagtagtaatataatatttcaaatatttttttcaaaataaaagaatgtagtatatagcaattgcttttctgtagtttataagtgtgtatattttaatttataacttttctaatatatgaccaaaatttgttgatgtgcagttgggaaattgggtt

SEQ ID NO: 26 - DNA, 57 bp, synthetic construct
  misc_feature 1..57: note = Description of Artificial Sequence: Synthetic oligonucleotide
  misc_feature 1..57: note = Codon optimized
  source 1..57: mol_type = other DNA
atggctaagttggttttttctctctgttttttgctcttttccggctgttgctttgca

SEQ ID NO: 27 - DNA, 63 bp, Arabidopsis thaliana
  source 1..63: mol_type = other DNA
atggactctaaaagtttcctcctgctgttgctccttttttgcttcttatttttgcacgacgca

SEQ ID NO: 28 - DNA, 221 bp, unidentified
  misc_feature 1..221: note = Description of Unknown: Agrobacterium tumefaciens sequence
  source 1..221: mol_type = other DNA
gaaccgcaacgttgaaggagccactgagccgcgggtttctggagtttaatgagctaagcacatacgtcagaaaccattattgcgcgttcaaaagtcgcctaaggtcactatcagctagcaaatatttcttgtcaaaaatgctccactgacgttccataaattcccctcggtatccaattagagtctcatattcactctcaactcgatcgaggggatctacc

SEQ ID NO: 29 - DNA, 403 bp, Cauliflower mosaic virus
  source 1..403: mol_type = other DNA
tgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgacatctccactgacgtaagggatgacgcacaatcccactatccttcgcaagacccttcctctatataaggaagttcatttcatttggagaggacaacaattaccaacaacaacaaacaacaaacaacattacaattactatttacaattaca

SEQ ID NO: 30 - DNA, 1014 bp, Glycine max
  source 1..1014: mol_type = other DNA
atcagaattaaactttaattctagttaattagaaaattttaggtttaaatacaacttcagtgatcttattttatttattctgtaattttagtctctttattttgaaataaaaattttgatccttcaattttaaaaaattcacaattaattttgatttcattttcaattttgtcatttatttattttatttcttatattttaattgaacaaataatttattgatgacactttaaatgaattttttaggtttaagattaagttaaattaaaataaaaagcataaaacataaataaaattgagaactaaactaaaattatattttttaaaataaaaaaatctctatttctgaaataggtgaactaaaattaccaatagaaaaaaataattaaatgataaacttttgaataatctcactaatcacttttaagatctcttattcaataaatttttcttttacattcatagaactcatatccgaaacctaaggaccgaatcaataccactcgatatgttgataaataataattattttaaaatctaaatctagttaaaataatttttatttggttgaaaatgttaatatctttataaaagtacagtattacaagaacaaaatgagaaagaaattgaaattcagtctaatttataaataatcaacctgcatgtaaaaggaaagaaagaagcgagcaggaagaaaagaaatgaaaccatgcatggtccccccacccccaggacatcatgggtttctgccatttgcaatacaaacactgaaacacctttctctttgtcacgtaatcgagattccgaagccaccttacaccattaacttaatgaggtgtaagacagaagggttccatagccatgcatactgaagaatgtcttaagctcagcaccccacttctgagacgtgtccctcattcaccttcctctcttccctataaataaccacgcctcaggttctccgcttcacaacacaaacattctctccatttgtccttgaatataatactcagc

SEQ ID NO: 31 - DNA, 403 bp, Cauliflower mosaic virus; identical to SEQ ID NO: 29
  source 1..403: mol_type = other DNA

SEQ ID NO: 32 - DNA, 382 bp, unidentified
  misc_feature 1..382: note = Description of Unknown: Agrobacterium tumefaciens sequence
  source 1..382: mol_type = other DNA
ttttcaaatcagtgcgcaagacgtgacgtaagtatccgagtcagtttttatttttctactaatttggtcgtttatttcggcgtgtaggacatggcaaccgggcctgaatttcgcgggtattctgtttctattccaactttttcttgatccgcagccattaacgacttttgaatagatacgctgacacgccaagcctcgctagtcaaaagtgtaccaaacaacgctttacagcaagaacggaatgcgcgtgacgctcgcggtgacgccatttcgccttttcagaaatggataaatagccttgcttcctattatatcttcccaaattaccaatacattacactagcatctgaatttcataaccaatctcgatacaccaaatcga

SEQ ID NO: 33 - DNA, 720 bp, synthetic construct
  misc_feature 1..720: note = Description of Artificial Sequence: Synthetic polynucleotide
  misc_feature 1..720: note = Original GFP sequence was from jellyfish. This has changes that increase the fluorescence of the protein as well as codon optimizations for expression in plants.
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccttcagctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactcacggcatggacgagctgtacaagtaa

SEQ ID NO: 34 - DNA, 552 bp, synthetic construct
  misc_feature 1..552: note = Description of Artificial Sequence: Synthetic polynucleotide
  misc_feature 1..552: note = Codon optimized
  source 1..552: mol_type = other DNA
atgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggtccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtgccgccccgtccggtcctgcccgtcaccgaaatctga

SEQ ID NO: 35 - DNA, 253 bp, unidentified
  misc_feature 1..253: note = Description of Unknown: Agrobacterium tumefaciens sequence
  source 1..253: mol_type = other DNA
gatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatc

SEQ ID NO: 36 - protein, 190 aa, Bos taurus
MMKSFFLVVTILALTLPFLGAQEQNQEQPIRCEKDERFFSDKIAKYIPIQYVLSRYPSYGLNYYQQKPVALINNQFLPYPYYAKPAAVRSPAQILQWQVLSNTVPAKSCQAQPTTMARHPHPHLSFMAIPPKKNQDKTEIPTINTIASGEPTSTPTTEAVESTVATLEDSPEVIESPPEINTVQVTSTAV

SEQ ID NO: 37 - protein, 214 aa, Bos taurus
MKLLILTCLVAVALARPKHPIKHQGLPQEVLNENLLRFFVAPFPEVFGKEKVNELSKDIGSESTEDQAMEDIKQMEAESISSSEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSEKTTMPLW

SEQ ID NO: 38 - protein, 222 aa, Bos taurus
MKFFIFTCLLAVALAKNTMEHVSSSEESIISQETYKQEKNMAINPSKENLCSTFCKEVVRNANEEEYSIGSSSEESAEVATEEVKITVDDKHYQKALNEINQFYQKFPQYLQYLYQGPIVLNPWDQVKRNAVPITPTLNREQLSTSEENSKKTVDMESTEVFTKKTKLTEEEKNRLNFLKKISQRYQKFALPQYLKTVYQHQKAMKPWIQPKTKVIPYVRYL

SEQ ID NO: 39 - protein, 224 aa, Bos taurus
MKVLILACLVALALARELEELNVPGEIVESLSSSEESITRINKKIEKFQSEEQQQTEDELQDKIHPFAQTQSLVYPFPGPIPNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRGPFPIIV

SEQ ID NO: 40 - protein, 536 aa, synthetic construct
  REGION 1..536: note = Description of Artificial Sequence: Synthetic polypeptide
MSSSFLSSTAFFLLLCLGFCHVSSQLRPRERPRGCPCTGRASSLARDSAAAASDPGTIVHNFSRTEPRTEPAGGSHSGSSSKLQALFAHPLYNVPEEPPLLGAEDSLLASQEALRYYRRKVARWNRRHKMYREQMNLTSLDPPLQLRLEASWVQFHLGINRHGLYSRSSPVVSKLLQDMRHFPTISADYSQDEKALLGACDCTQIVKPSGVHLKLVLRFSDFGKAMFKPMRQQRDEETPVDFFYFIDFQRHNAEIAAFHLDRILDFRRVPPTVGRIVNVTKEILEVTKNEILQSVFFVSPASNVCFFAKCPYMCKTEYAVCGNPHLLEGSLSAFLPSLNLAPRLSVPNPWIRSYTLAGKEEWEVNPLYCDTVKQIYPYNNSQRLLNVIDMAIFDFLIGNMDRHHYEMFTKFGDDGFLIHLDNARGFGRHSHDEISILSPLSQCCMIKKKTLLHLQLLAQADYRLSDVMRESLLEDQLSPVLTEPHLLALDRRLQTILRTVEGCIVAHGQQSVIVDGPVEQLAPDSGQANLTSHDEL

SEQ ID NO: 41 - protein, 586 aa, synthetic construct
  REGION 1..586: note = Description of Artificial Sequence: Synthetic polypeptide
MSSSFLSSTAFFLLLCLGFCHVSSLDLLPRLERRGARPSGEPGCSCAQPAAEVAAPGWAQVRGRPGEPPAASSAAGDAGWPNKHTLRILQDFSSDPSSNLSSHSLEKLPPAAEPAERALRGRDPGALRPHDPAHRPLLRDPGPRRSESPPGPGGDASLLARLFEHPLYRVAVPPLTEEDVLFNVNSDTRLSPKAAENPDWPHAGAEGAEFLSPGEAAVDSYPNWLKFHIGINRYELYSRHNPAIEALLHDLSSQRITSVAMKSGGTQLKLIMTFQNYGQALFKPMKQTREQETPPDFFYFSDYERHNAEIAAFHLDRILDFRRVPPVAGRMVNMTKEIRDVTRDKKLWRTFFISPANNICFYGECSYYCSTEHALCGKPDQIEGSLAAFLPDLSLAKRKTWRNPWRRSYHKRKKAEWEVDPDYCEEVKQTPPYDSSHRILDVMDMTIFDFLMGNMDRHHYETFEKFGNETFIIHLDNGRGFGKYSHDELSILVPLQQCCRIRKSTYLRLQLLAKEEYKLSLLMAESLRGDQVAPVLYQPHLEALDRRLRVVLKAVRDCVERNGLHSVVDDDLDTEHRAASARHDEL

SEQ ID NO: 42 - DNA, 426 bp, Cauliflower mosaic virus
  source 1..426: mol_type = other DNA
gtcaacatggtggagcacgacactctggtctactccaaaaatgtcaaagatacagtctcagaagatcaaagggctattgagacttttcaacaaaggataatttcgggaaacctcctcggattccattgcccagctatctgtcacttcatcgaaaggacagtagaaaaggaaggtggctcctacaaatgccatcattgcgataaaggaaaggctatcattcaagatctctctgccgacagtggtcccaaagatggacccccacccacgaggagcatcgtggaaaaagaagaggttccaaccacgtctacaaagcaagtggattgatgtgacatctccactgacgtaagggatgacgcacaatcccactatccttcgcaagacccttcctctatataaggaagttcatttcatttggagaggacacgc

SEQ ID NO: 43 - protein, 391 aa, Homo sapiens
MSGPVPSRARVYTDVNTHRPREYWDYESHVVEWGNQDDYQLVRKLGRGKYSEVFEAINITNNEKVVVKILKPVKKKKIKREIKILENLRGGPNIITLADIVKDPVSRTPALVFEHVNNTDFKQLYQTLTDYDIRFYMYEILKALDYCHSMGIMHRDVKPHNVMIDHEHRKLRLIDWGLAEFYHPGQEYNVRVASRYFKGPELLVDYQMYDYSLDMWSLGCMLASMIFRKEPFFHGHDNYDQLVRIAKVLGTEDLYDYIDKYNIELDPRFNDILGRHSRKRWERFVHSENQHLVSPEALDFLDKLLRYDHQSRLTAREAMEHPYFYTVVKDQARMGSSSMPGGSTPVSSANMMSGISSVPTPSPLGPLAGSPVIAAANPLGMPVPAAAGAQQ

SEQ ID NO: 44 - DNA, 714 bp, unidentified
  misc_feature 1..714: note = Description of Unknown: Agrobacterium tumefaciens sequence
  source 1..714: mol_type = genomic DNA
gtcctgctttaatgagatatgcgagaagcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggccgggtaaccgacttgctgccccgagaattatgcagcatttttttggtgtatgtgggccccaaatgaagtgcaggtcaaaccttgacagtgacgacaaatcgttgggcgggtccagggcgaattttgcgacaacatgtcgaggctcagcaggac

SEQ ID NO: 45 - protein, 386 aa, Gallus gallus
MGSIGAASMEFCFDVFKELKVHHANENIFYCPIAIMSALAMVYLGAKDSTRTQINKVVRFDKLPGFGDSIEAQCGTSVNVHSSLRDILNQITKPNDVYSFSLASRLYAEERYPILPEYLQCVKELYRGGLEPINFQTAADQARELINSWVESQTNGIIRNVLQPSSVDSQTAMVLVNAIVFKGLWEKAFKDEDTQAMPFRVTEQESKPVQMMYQIGLFRVASMASEKMKILELPFASGTMSMLVLLPDEVSGLEQLESIINFEKLTEWTSSNVMEERKIKVYLPRMKMEEKYNLTSVLMAMGITDVFSSSANLSGISSAESLKISQAVHAAHAEINEAGREVVGSAEAGVDAASVSEEFRADHPFLFCIKHIATNAVLFFGRCVSP

SEQ ID NO: 46 - protein, 705 aa, Gallus gallus
MKLILCTVLSLGIAAVCFAAPPKSVIRWCTISSPEEKKCNNLRDLTQQERISLTCVQKATYLDCIKAIANNEADAISLDGGQAFEAGLAPYKLKPIAAEVYEHTEGSTTSYYAVAVVKKGTEFTVNDLQGKTSCHTGLGRSAGWNIPIGTLLHRGAIEWEGIESGSVEQAVAKFFSASCVPGATIEQKLCRQCKGDPKTKCARNAPYSGYSGAFHCLKDGKGDVAFVKHTTVNENAPDQKDEYELLCLDGSRQPVDNYKTCNWARVAAHAVVARDDNKVEDIWSFLSKAQSDFGVDTKSDFHLFGPPGKKDPVLKDLLFKDSAIMLKRVPSLMDSQLYLGFEYYSAIQSMRKDQLTPSPRENRIQWCAVGKDEKSKCDRWSVVSNGDVECTVVDETKDCIIKIMKGEADAVALDGGLVYTAGVCGLVPVMAERYDDESQCSKTDERPASYFAVAVARKDSNVNWNNLKGKKSCHTAVGRTAGWVIPMGLIHNRTGTCNFDEYFSEGCAPGSPPNSRLCQLCQGSGGIPPEKCVASSHEKYFGYTGALRCLVEKGDVAFIQHSTVEENTGGKNKADWAKNLQMDDFELLCTDGRRANVMDYRECNLAEVPTHAVVVRPEKANKIRDLLERQEKRFGVNGSEKSKFMMFESQNKDLLFKDLTKCLFKVREGTTYKEFLGDKFYTVISSLKTCNPSDILQMCSFLEGK

SEQ ID NO: 47 - protein, 210 aa, Gallus gallus
MAMAGVFVLFSFVLCGFLPDAAFGAEVDCSRFPNATDKEGKDVLVCNKDLRPICGTDGVTYTNDCLLCAYSIEFGTNISKEHDGECKETVPMNCSSYANTTSEDGKVMVLCNRAFNPVCGTDGVTYDNECLLCAHKVEQGASVDKRHDGGCRKELAAVSVDCSEYPKPDCTAEDRPLCGSDNKTYGNKCNFCNAVVESNGTLTLSHFGKC

SEQ ID NO: 48 - protein, 439 aa, Gallus gallus
MGALLALLDPVQPTRAPDCGGILTPLGLSYLAEVSKPHAEVVLRQDLMAQRASDLFLGSMEPSRNRITSVKVADLWLSVIPEAGLRLGIEVELRVAPLHAVPMPVRISIRADLHVDMGPDGNLQLLTSACRPTVQAQSTREAESKSSRSILDKVVDVDKLCLDVSKLLLFPNEQLMSLTALFPVTPNCQLQYLPLAAPVFSKQGIALSLQTTFQVAGAVVPVPVSPVPFSMPELASTSTSHLILALSEHFYTSLYFTLERAGAFNMTIPSMLTTATLAQKITQVGSLYHEDLPITLSAALRSSPRVVLEEGRAALKLFLTVHIGAGSPDFQSFLSVSADVTAGLQLSVSDTRMMISTAVIEDAELSLAASNVGLVRAALLEELFLAPVCQQVPAWMDDVLREGVHLPHLSHFTYTDVSVVVHKDYVLVPCKLKLRSTMA

SEQ ID NO: 49 - protein, 1463 aa, Bos taurus
MFSFVDLRLLLLLAATALLTHGQEEGQEEGQEEDIPPVTCVQNGLRYHDRDVWKPVPCQICVCDNGNVLCDDVICDELKDCPNAKVPTDECCPVCPEGQESPTDQETTGVEGPKGDTGPRGPRGPAGPPGRDGIPGQPGLPGPPGPPGPPGPPGLGGNFAPQLSYGYDEKSTGISVPGPMGPSGPRGLPGPPGAPGPQGFQGPPGEPGEPGASGPMGPRGPPGPPGKNGDDGEAGKPGRPGERGPPGPQGARGLPGTAGLPGMKGHRGFSGLDGAKGDAGPAGPKGEPGSPGENGAPGQMGPRGLPGERGRPGAPGPAGARGNDGATGAAGPPGPTGPAGPPGFPGAVGAKGEGGPQGPRGSEGPQGVRGEPGPPGPAGAAGPAGNPGADGQPGAKGANGAPGIAGAPGFPGARGPSGPQGPSGPPGPKGNSGEPGAPGSKGDTGAKGEPGPTGIQGPPGPAGEEGKRGARGEPGPAGLPGPPGERGGPGSRGFPGADGVAGPKGPAGERGAPGPAGPKGSPGEAGRPGEAGLPGAKGLTGSPGSPGPDGKTGPPGPAGQDGRPGPPGPPGARGQAGVMGFPGPKGAAGEPGKAGERGVPGPPGAVGPAGKDGEAGAQGPPGPAGPAGERGEQGPAGSPGFQGLPGPAGPPGEAGKPGEQGVPGDLGAPGPSGARGERGFPGERGVQGPPGPAGPRGANGAPGNDGAKGDAGAPGAPGSQGAPGLQGMPGERGAAGLPGPKGDRGDAGPKGADGAPGKDGVRGLTGPIGPPGPAGAPGDKGEAGPSGPAGPTGARGAPGDRGEPGPPGPAGFAGPPGADGQPGAKGEPGDAGAKGDAGPPGPAGPAGPPGPIGNVGAPGPKGARGSAGPPGATGFPGAAGRVGPPGPSGNAGPPGPPGPAGKEGSKGPRGETGPAGRPGEVGPPGPPGPAGEKGAPGADGPAGAPGTPGPQGIAGQRGVVGLPGQRGERGFPGLPGPSGEPGKQGPSGASGERGPPGPMGPPGLAGPPGESGREGAPGAEGSPGRDGSPGAKGDRGETGPAGPPGAPGAPGAPGPVGPAGKSGDRGETGPAGPAGPIGPVGARGPAGPQGPRGDKGETGEQGDRGIKGHRGFSGLQGPPGPPGSPGEQGPSGASGPAGPRGPPGSAGSPGKDGLNGLPGPIGPPGPRGRTGDAGPAGPPGPPGPPGPPGPPSGGYDLSFLPQPPQEKAHDGGRYYRADDANVVRDRDLEVDTTLKSLSQQIENIRSPEGSRKNPARTCRDLKMCHSDWKSGEYWIDPNQGCNLDAIKVFCNMETGETCVYPTQPSVAQKNWYISKNPKEKRHVWYGESMTGGFQFEYGGQGSDPADVAIQLTFLRLMSTEASQNITYHCKNSVAYMDQQTGNLKKALLLQGSNEIEIRAEGNSRFTYSVTYDGCTSHTGAWGKTVIEYKTTKTSRLPIIDVAPLDVGAPDQEFGFDVGPACFL

SEQ ID NO: 50 - DNA, 1341 bp, Glycine max
  source 1..1341: mol_type = other DNA
ggttcaacaacacaagcttcaagttttaaaaggaaaaatgtcagccaaaaactttaaataaaatggtaacaaggaaattattcaaaaattacaaacctcgtcaaaataggaaagaaaaaaagtttagggatttagaaaaaacatcaatctagttccaccttattttatagagagaagaaactaatatataagaactaaaaaacagaagaatagaaaaaaaaagtattgacaggaaagaaaaagtagctgtatgcttataagtactttgaggatttgaattctctcttataaaacacaaacacaatttttagattttatttaaataatcatcaatccgattataattatttatatatttttctattttcaaagaagtaaatcatgagcttttccaactcaacatctattttttttctctcaacctttttcacatcttaagtagtctcaccctttatatatataacttatttcttaccttttacattatgtaacttttatcaccaaaaccaacaactttaaaattttattaaatagactccacaagtaacttgacactcttacattcatcgacattaacttttatctgttttataaatattattgtgatataatttaatcaaaataaccacaaactttcataaaaggttcttattaagcatggcatttaataagcaaaaacaactcaatcactttcatataggaggtagcctaagtacgtactcaaaatgccaacaaataaaaaaaaagttgctttaataatgccaaaacaaattaataaaacacttacaacaccggattttttttaattaaaatgtgccatttaggataaatagttaatatttttaataattatttaaaaagccgtatctactaaaatgatttttatttggttgaaaatattaatatgtttaaatcaacacaatctatcaaaattaaactaaaaaaaaaataagtgtacgtggttaacattagtacagtaatataagaggaaaatgagaaattaagaaattgaaagcgagtctaatttttaaattatgaacctgcatatataaaaggaaagaaagaatccaggaagaaaagaaatgaaaccatgcatggtcccctcgtcatcacgagtttctgccatttgcaatagaaacactgaaacacctttctctttgtcacttaattgagatgccgaagccacctcacaccatgaacttcatgaggtgtagcacccaaggcttccatagccatgcatactgaagaatgtctcaagctcagcaccctacttctgtgacgtgtccctcattcaccttcctctcttccctataaataaccacgcctcaggttctccgcttc

SEQ ID NO: 51 - protein, 147 aa, Gallus gallus
MRSLLILVLCFLPLAALGKVFGRCELAAAMKRHGLDNYRGYSLGNWVCAAKFESNFNTQATNRNTDGSTDYGILQINSRWWCNDGRTPGSRNLCNIPCSALLSSDITASVNCAKKIVSDGNGMNAWVAWRNRCKGTDVQAWIRGCRL

SEQ ID NO: 52 - protein, 517 aa, Gallus gallus
MTDWVLHHKVGPLDMTTRYIFPLLPLPFLPHSESKRAVCAPRCSAMRTARQFVQVALALCCFADIAFGIEVNCSLYASGIGKDGTSWVACPRNLKPVCGTDGSTYSNECGICLYNREHGANVEKEYDGECRPKHVTIDCSPYLQVVRDGNTMVACPRILKPVCGSDSFTYDNECGICAYNAEHHTNISKLHDGECKLEIGSVDCSKYPSTVSKDGRTLVACPRILSPVCGTDGFTYDNECGICAHNAEQRTHVSKKHDGKCRQEIPEIDCDQYPTRKTTGGKLLVRCPRILLPVCGTDGFTYDNECGICAHNAQHGTEVKKSHDGRCKERSTPLDCTQYLSNTQNGEAITACPFILQEVCGTDGVTYSNDCSLCAHNIELGTSVAKKHDGRCREEVPELDCSKYKTSTLKDGRQVVACTMIYDPVCATNGVTYASECTLCAHNLEQRTNLGKRKNGRCEEDITKEHCREFQKVSPICTMEYVPHCGSDGVTYSNRCFFCNAYVQSNRTLNLVSMAAC

SEQ ID NO: 53 - protein, 225 aa, Gallus gallus
VVSTAAPTDPRRRMAVSVLLFISVALAGVLSTASQACKLEPVKIDLANPQLAGKWYFIQVATEVELYQARFANIDNSYFISTPDVKLNKTNIKEYSQLGDLCLSTNTDYVVLENGYEFTDGAKNINNFRIIKSKIDNMLIIDYQYQIEKMDYHMLTLFKRNPTASKEEMEIFESYTKCLGLDKDKIKAFPRNKSECKEEKQINSFNATTQAQDFLEEKVLQNRNI

SEQ ID NO: 54 - DNA, 886 bp, Glycine max
  source 1..886: mol_type = other DNA
gtgtgttgtaagtataaatttaaaataaaaataaaaacaattattatatcaaaatggcaaaaacatttaatacgtattatttaagaaaaaaatatgtaataatatatttatattttaatatctattcttatgtattttttaaaaatctattatatattgatcaactaaaatatttttatatctacacttattttgcatttttatcaattttcttgcgttttttggcatatttaataatgactattctttaataatcaatcattattcttacatggtacatattgttggaaccatatgaagtgtccattgcatttgactatgtggatagtgttttgatccaggcctccatttgccgcttattaattaatttggtaacagtccgtactaatcagttacttatccttcctccatcataattaatcttggtagtctcgaatgccacaacactgactagtctcttggatcataagaaaaagccaaggaacaaaagatcacaaaacacaatgagagtatcctttgcatagcaatgtctaagttcataaaattcaaacaaaaacgcaatcacacacagtggacatcacttatccactagctgatcaggatcgccgcgtcaagaaaaaaaaactggaccccaaaagccatgcacaacaacacgtactcacaaaggtgtcaatcgagcagcccaaaacattcaccaactcaacccatcatgagcccacacatttgttgtttctaacccaacctcaaactcgtattctcttccgccacctcatttttgtttatttcaacacccgtcaaactgcatgccaccccgtggccaaatgtccatgcatgttaacaagacctatgactataaatatctgcaatctcggcccaggttttcatc

SEQ ID NO: 55 - protein, 208 aa, Gallus gallus
ALTSGLGPDVYQFLQDMGMKFFTNSKIRQPTVCTRETVRPPSYFLNAGFTASTHHVKLSAEVAREERGKRHILETIREFFPETWIWDIILINSTGKASVSYTIPDTITEWKASAFCVLPPNVVEESARASVSVLGDILGSAMQNTQNLLQMPYGCGEQNMVLFAPNIYVLDYLNETQQLSEDMKSKTIGYLESGYQKQLSYKHPDGSY

SEQ ID NO: 56 - protein, 152 aa, Gallus gallus
MVHATSPLLLLLLLSLALVAPGLSARKCSLTGKWTNDLGSNMTIGAVNSRGEFTGTYITAVTATSNEIKESPLHGTQNTINKRTQPTFGFTVNWKFSESTTVFTGQCFIDRNGKEVLKTMWLLRSSVNDIGDDWKATRVGINIFTRLRTQKE

SEQ ID NO: 57 - protein, 148 aa, Gallus gallus
MVGSPRAPLLLLASLIVALALALAVSPAAAQGPRKGRLLGGLMEADVNEEGVQEALSFAVSEFNKRSNDAYQSRVVRVVRARKQVVSGMNYFLDVELGRTTCTKSQANLDSCPFHNQPHLKREKLCSFQVYVVPWMNTINLVKFSCQD

SEQ ID NO: 58 - DNA, 62 bp, Tobacco mosaic virus
  source 1..62: mol_type = other DNA
atttttacaacaattaccaacaacaacaaacaacaaacaacattacaattacatttacaatt

SEQ ID NO: 59 - DNA, 739 bp, Arabidopsis thaliana
  source 1..739: mol_type = other DNA
ttgcttcttctctctttttttggttcaatatgaaccttttgatgtccactatccttttatataatgaaatgataaggtttttgatatgttatgtggttcttgataacattatacaattacttaatatctacatatgaaaggttggaattttttttaagtcaccacaatagaggtgacacgtgtaagcacctcgttaatcttatctcatccaagatgggggtaggaagaggatatgaatgtatgattgaggttggttttgagttttttttttttttttttttgagtccttcaacttgttattttaatttttttttggtgggggaggaggggggttgaaatatttatcatatagtagtccaaagtaaattgatagctagagtacttgtttgcttgcttatattgtcctcaactttatgtaataccatgattccaacttagacactcttttaagttgtaattttcattattttctttttttagagttttatgttgaattcgcataattttcaatcggataatacaagaaaaataatattttagtataattttatacatgaaatttcgggaaggtaggatatacggattgtttgtcggatcagagactttactcgtacctttgtaactgttgatcccaaatacagatagtgactccagagttatatattatacctatagagactaatatgatttagtgttattaaaattagtattactaattaattgtaatgccccatgaatt

SEQ ID NO: 60 - DNA, 4968 bp, synthetic construct
  misc_feature 1..4968: note = Description of Artificial Sequence: Synthetic polynucleotide
  source 1..4968: mol_type = other DNA
tctgtgaagacaaactagaattcgagctcggagtgagaccgcagctggcacgacaggtttgccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaggatccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgacggtctcacgctttacttgtcttctgcacgaagtggtttaaactatcagtgtttgacaggatatattggcgggtaaacctaagagaaaagagcgtttattagaataatcggatatttaaaagggcgtgaaaaggtttatccgttcgtccatttgtatgtgcatgccaaccacagggttccccagatcaggcgctggctgctgaacccccagccggaactgaccccacaaggccctagcgtttgcaatgcaccaggtcatcattgacccaggcgtgttccaccaggccgctgcctcgcaactcttcgcaggcttcgccgacctgctcgcgccacttcttcacgcgggtggaatccgatccgcacatgaggcggaaggtttccagcttgagcgggtacggctcccggtgcgagctgaaatagtcgaacatccgtcgggccgtcggcgacagcttgcggtacttctcccatatgaatttcgtgtagtggtcgccagcaaacagcacgacgatttcctcgtcgatcaggacctggcaacgggacgttttcttgccacggtccaggacgcggaagcggtgcagcagcgacaccgattccaggtgcccaacgcggtcggacgtgaagcccatcgccgtcgcctgtaggcgcgacaggcattcctcggccttcgtgtaataccggccattgatcgaccagcccaggtcctggcaaagctcgtagaacgtgaaggtgatcggctcgccgataggggtgcgcttcgcgtactccaacacctgctgccacaccagttcgtcatcgtcggcccgcagctcgacgccggtgtaggtgatcttcacgtccttgttgacgtggaaaatgaccttgttttgcagcgcctcgcgcgggattttcttgttgcgcgtggtgaacagggcagagcgggccgtgtcgtttggcatcgctcgcatcgtgtccggccacggcgcaatatcgaacaaggaaagctgcatttccttgatctgctgcttcgtgtgtttcagcaacgcggcctgcttggcctcgctgacctgttttgccaggtcctcgccggcggtttttcgcttcttggtcgtcatagttcctcgcgtgtcgatggtcatcgacttcgccaaacctgccgcctcctgttcaagacgacgcgaacgctccacggcggccgatggcgcgggcagggcagggggagccagttgcacgctgtcgcgctcgatcttggccgtagcttgctggaccatcgagccgacggactggaaggtttcgcggggcgcacgcatgacggtgcggcttgcgatggtttcggcatcctcggcggaaaaccccgcgtcgatcagttcttgcctgtatgccttccggtcaaacgtccgattcattcaccctccttgcgggattgccccgactcacgccggggcaatgtgcccttattcctgatttgacccgcctggtgccttggtgtccagataatccaccttatcggcaatgaagtcggtcccgtagaccgtctggccgtccttctcgtacttggtattccgaatcttgccctgcacgaataccagcgaccccttgcccaaatacttgccgtgggcctcggcctgagagccaaaacacttgatgcggaagaagtcggtgcgctcctgcttgtcgccggcatcgttgcgccacatctaggatctgccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagaaccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacgaattggccagcgctgccatttttggggtgaggccgttcgcggccgaggggcgcagcccctggggggatgggaggcccgcgttagcgggccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccggtgagcgtcgcaaaggagatcctgatctgactgatgggctgcctgtatcgagtggtgattttgtgccgagctgccggtcggggagctgttggctggctggtggcaggatatattgtggtgtaaacaaattgacgcttagacaacttaataacacattgcggacgtttttaatgtactggggtggatgcagtgggccccac

SEQ ID NO: 61 - DNA, 790 bp, synthetic construct
  misc_feature 1..790: note = Description of Artificial Sequence: Synthetic polynucleotide
  source 1..790: mol_type = other DNA
gccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctg

SEQ ID NO: 62 - DNA, 618 bp, synthetic construct
  misc_feature 1..618: note = Description of Artificial Sequence: Synthetic polynucleotide
  source 1..618: mol_type = other DNA
ccgggagggttcgagaagggggggcaccccccttcggcgtgcgcggtcacgcgcacagggcgcagccctggttaaaaacaaggtttataaatattggtttaaaagcaggttaaaagacaggttagcggtggccgaaaaacgggcggaaacccttgcaaatgctggattttctgcctgtggacagcccctcaaatgtcaataggtgcgcccctcatctgtcagcactctgcccctcaagtgtcaaggatcgcgcccctcatctgtcagtagtcgcgcccctcaagtgtcaataccgcagggcacttatccccaggcttgtccacatcatctgtgggaaactcgcgtaaaatcaggcgttttcgccgatttgcgaggctggccagctccacgtcgccggccgaaatcgagcctgcccctcatctgtcaacgccgcgccgggtgagtcggcccctcaagtgtcaacgtccgcccctcatctgtcagtgagggccaagttttccgcgaggtatccacaacgccggcggccgcggtgtctcgcacacggcttcgacggcgtttctggcgcgtttgcagggccatagacggccgccagcccagcggcgagggcaaccagcccgg

SEQ ID NO: 63 - DNA, 537 bp, synthetic construct
  misc_feature 1..537: note = Description of Artificial Sequence: Synthetic polynucleotide
  source 1..537: mol_type = other DNA
ccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcttgcatgcctgcaggtcgactctagaggatccccgggtaccgagctcgaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatcc

SEQ ID NO: 64 - DNA, 128 bp, synthetic construct
  misc_feature 1..128: note = Description of Artificial Sequence: Synthetic polynucleotide
  source 1..128: mol_type = other DNA
aaactatcagtgtttgacaggatatattggcgggtaaacctaagagaaaagagcgtttattagaataatcggatatttaaaagggcgtgaaaaggtttatccgttcgtccatttgtatgtgcatgcca

SEQ ID NO: 65 - DNA, 15 bp, synthetic construct
  misc_feature 1..15: note = Description of Artificial Sequence: Synthetic oligonucleotide
  source 1..15: mol_type = other DNA
cacgacgaattgtaa

SEQ ID NO: 66 - DNA, 1674 bp, synthetic construct
  misc_feature 1..1674: note = Description of Artificial Sequence: Synthetic polynucleotide
  source 1..1674: mol_type = other DNA
ctagatttactcccccgactggagcgacgtggagcgcgtccgtctggcgaaccaggatgttcttgtgcgcaaccagctgcggaagtcgctgcgccaggttgggcacaagttcgagggaggcctggggagccaccagctgctagctccgctgcgggcgatgccggatggccaaataaacacactctaagaatactacaggacttctcaagcgacccttcctctaatctctcatcacactcacttgagaagttacctcccgcagccgagccggccgagcgagcgcttagggggcgtgaccccggggcattacgacctcacgatcctgcacacaggcctttgttacgtgatccagggccgcgaagaagcgaatcccctcctggacccggaggcgacgcatcccttctggcgaggttatttgaacacccattgtacagagtcgccgtgcctcccctgactgaagaggacgtcttgttcaacgttaactctgacaccagacttagtcctaaagcggccgagaatcctgattggccgcatgccggggcagagggcgctgagttccttagcccaggagaggccgcggtggactcctatccgaactggttgaaattccatattggaataaatcgatacgaactatattctcgacacaatccggccatcgaggccctattacatgatctgtcctcacaacgtattacgtcagtggccatgaagagcggcggcacgcaactaaagctgattatgacatttcaaaactatggtcaagccctgtttaagccaatgaagcagacaagagaacaggagacgcctcccgacttcttctactttagtgattatgaacgacataatgctgaaatagcggcctttcacctagacaggatcttagacttccgtagggttcccccagttgcaggtaggatggttaacatgactaaagagataagggatgtaaccagggataagaagctatggaggacgttctttatttctcctgcgaacaatatctgtttttacggcgagtgctcctactattgcagcacagaacatgcactatgtggaaagcctgaccagattgaaggttccctggcggcctttcttccagacctctctcttgcgaaacgtaaaacttggcgtaatccatggagacgaagctatcacaaacgtaagaaagcggagtgggaggtagaccccgattactgcgaggaggttaagcaaaccccgccgtatgactctagccaccgaatattagacgttatggatatgacgatctttgattttcttatgggcaacatggatcgtcaccactacgaaacgttcgagaagtttggaaatgagacgtttattatccatctggacaacggcagaggctttgggaaatatagtcatgacgaattatccattcttgttcctttacagcaatgctgcagaatcaggaaatcaacgtatttacgtctccaactcctggcaaaagaggaatataagcttagcctcctaatggctgagtctctgaggggcgaccaagttgcgcctgttctgtatcaaccacatttagaagcacttgatagacgtttgcgtgttgtacttaaagctgtccgagactgtgtcgagagaaatggccttcattcagttgtggacgacgatctagataccgaacatagggcagcgtccgcacgt

SEQ ID NO: 67 - DNA, 1524 bp, synthetic construct
  misc_feature 1..1524: note = Description of Artificial Sequence: Synthetic polynucleotide
  source 1..1524: mol_type = other DNA
caactcaggccaagggaacgtccccgtgggtgcccgtgtaccggaagggcatctagtttggcgcgagattctgctgctgcggcgtcagacccagggacaattgtgcataatttttcccgtactgagccaagaacagagccggccggtgggagccatagcggaagttcatctaagttgcaagcactattcgcgcatcctttgtataatgtacctgaagaacctcctcttttaggagcggaggactcactcttagccagccaggaagcccttagatactaccgtagaaaggttgcccgatggaaccgtaggcataaaatgtaccgtgagcaaatgaatttaactagtcttgatcctcctctccaactgaggcttgaggctagttgggttcagttccatttgggtatcaatcgacacggcctttactctaggtcaagtcctgtggtatctaaacttcttcaagacatgcgtcattttccgacaatatctgccgattactcccaggatgagaaggctttgcttggagcatgtgactgtacccagatagtaaaacctagtggcgtccacttaaaactggtcctccgttttagcgattttgggaaagctatgtttaaacccatgcgacaacaacgtgatgaagaaacgccagttgatttcttctacttcattgacttccagcgtcacaacgcggagatagcagcattccacttagacagaattctagactttcgtcgagtcccccctaccgttggccgtatagtgaatgtaactaaagagattttggaggtgacaaaaaacgagatcttacaaagcgtcttttttgtatccccggcctccaacgtctgtttttttgcgaaatgtccctatatgtgcaaaactgaatatgcggtctgcggcaacccccatttactcgaaggtagtctcagtgcatttctccccagtctcaacttagctccacgtctaagcgtgccaaacccttggattaggagctatacgctagcgggcaaggaggagtgggaggtgaacccactttattgtgacacagtcaagcaaatctatccttataataactcacagagactactcaatgtcattgatatggctatcttcgatttcctgataggaaacatggataggcaccattatgagatgttcaccaagttcggggacgatggttttctgatacatctagacaacgcgcgtggcttcgggcgacactcccacgacgaaatctccattcttagccccctgagccagtgttgcatgatcaaaaagaaaacacttctgcatcttcagctcctcgctcaagctgactatcgactttccgacgtgatgcgagaatcactgcttgaagatcagctcagcccagtgcttactgaaccgcacctactggcactagatcgtagattacagacgatcttgaggacagtcgaggggtgcatcgttgctcacggccaacaaagcgtcatagttgatgggcctgtggaacagctagctccggattccggacaagcaaatttaaccagc

SEQ ID NO: 68 - DNA, 1674 bp, synthetic construct
  misc_feature 1..1674: note = Description of Artificial Sequence: Synthetic polynucleotide
  source 1..1674: mol_type = other DNA
ctcctacccaaattagaacgatctgctgcacgtccgagcggcgaacctggctgtagctgcgcacagccagctgccgaagctgccgcgcctggatgggctcaagctaggggtcatcccggtggagaacttgaagcggccgctagcgccgccggggatgcaggctggccaaataagcacactctgaggattctgcaagacttcagttccgaccccagttccaacctaacgagccactcactggaaaagctgcctccggctgccgaagctgcggaaggtgcaccgccaggccaagatccaggagttcgtagacctcccgacccagcgcataggccactcccgcgagatccgggtcctagaggccctgtcttgcccccaggtcttagcggagacgggtccttacttacgcgtcttttccaacacccgctataccaggtgcccataccgcccctaacagaaggcgatgttctctttaatgtcaatagcgatataagattcaaccccaaagctgcaaccgccgagaacccagattggccacacgaggggccggaagatgagtttttacctactggtgaagcggcagttgactcttacccgaattggctgaagtttcatattgggatcaatagatacgagctttacagccgacataatccggccgtgggagcgctcttacaagacctcgggacgcaaaagattacttctgtcgctatgaaatctggcgggacacagctcaaacttattatgactttccagaattatggccaagctctgttcaagccgatgaagcagactagagagcaggagacaccccctgacttcttctacttcagcgactatgaaaggcataatgcagaaattgcggcattccaccttgataggatcttagacttccgaagggtaccaccggtagcaggtagactagtcaatatgactaaagagattagagatgtcactcgtgacaagaaactatggcgtacattctttataagccctgctaacaatgtatgcttttatggcgaatgttcttactattgctctacagaacatgcactgtgtggaaaacccgaccagattgaggggtcactagccgcatttctgccagacttggcattggccaagcgtaagacgtggcgtaatccgtggcgacgtagttaccacaagagaaagaaggcggagtgggaagtagacccagactactgcgaggaggttagacaaacacctccatatgattctagtcatagactgttggatgttatggacatgacaatttttgattttctcatggggaacatggatcgtcaccactacgaaacctttgagaaattcggcaatgagacattcattatccacttagataatggtcgaggttttggcaaacacagccatgacgaactatctatattagtgcctttacaacagtgctgtagaatccgaaggtctacctatttgagacttcaactgttggcccaagaggagcatcgtctatcacttttaatggccgaggctctaagggctgatcgtgtggctcccgtactctttcagcctcacttagaggctttagatcgtcgacttcgtatagtgcttcgagcggtaggcgattgcgtggagaaagatggactgcacagtgttgtagaggatgatttggggcctgagcacagggcggccgcgggacgt

SEQ ID NO: 69 - DNA, 1503 bp, synthetic construct
  misc_feature 1..1503: note = Description of Artificial Sequence: Synthetic polynucleotide
  source 1..1503: mol_type = other DNA
caccttggtcccagggtacgatccagactgcaaccgagggaacgaccgttggggtgcccttgtgcgcgtcgtgccgctagtcctgctccgggacctgccccgagtgcgcccaggcgtgtggaaccaagcggcggtggcgatccagggtccaaactcagggcacttttcgcacacccattgtataacgaaccagaggaacctccgctgcttgggcccgaggatagcctgctggcgggtcccgaagcattacgttattaccgaagaaaagtagcaaggtggaatagacgtcgaaagatgtacaaagaacaattaaacctaaccagcccagaaccgccggtgcaattgagacaagaggcatcatgggtacaatttcacttggggatcaataggcatgggctgtatcctcgtagctctccggtggtttctaaactccttcaggatatgagacacttccctacgatcagcgcggattatagccaggacgaaaaggctcttcttggcgcatgtgactgttctcaaattgttaagccctccggcgttcatctgaagctcgtacttcgtttttctgattttgggaaggcgatgtttaaacccatgagacagcagagggacgaggagacgccggaggacttcttctatttcatcgacttccagagacataatgccgaaattgctgccttccatctagatcgtattttggactttagacgtgtcccgccaacagtagggaggctcgtaaatgtgacaagggaaatactagaagtcactcgtaatgagattttacaaagcgtgtttttcgtgagtccggcaaacaatgtgtgcttctttgcgaaatgcccttatatgtgcaagacggagtatgctgtttgcgggtcaccccacctactagaaggttctttaagtgccttccttcccagcctcaacctcgctccacgtttcagtatgcctagtccatggatcaggtcatattccctagctggtcgtgaagagtgggaggtaaacccgctatactgcgaagcggtgaaacaggcttacccccacaatagcagttcacgtctattaaatattatcgatatggcgatcttcgactttctaatagggaacatggacaggcaccactacgaaatgttcactaagttcggtgaggacgggttcctaatacatttggataatgcccgtggttttgggagacattcacacgatgaagtctctattttagcccctttattccaatgttgtaggataaaaagaaaaaccctgctgcatcttcaactcctggctcaggctgattaccgtctttcagacgttatgcgtgagtctttgctagaggaccaattgagtcctgtactaacagaaccacatctgctagctttggacagacgtttgcagactgtgctaagaacggtccaggattgcatcgaggcccatggggaacaatcagtggtagccgacggcccggtgggccaatgggctcccgacagtagtagagcgaacgcaacttct

SEQ ID NO: 70 - DNA, 383 bp, unidentified
  misc_feature 1..383: note = Description of Unknown: Agrobacterium tumefaciens sequence
  source 1..383: mol_type = other DNA
atttttcaaatcagtgcgcaagacgtgacgtaagtatccgagtcagtttttatttttctactaatttggtcgtttatttcggcgtgtaggacatggcaaccgggcctgaatttcgcgggtattctgtttctattccaactttttcttgatccgcagccattaacgacttttgaatagatacgctgacacgccaagcctcgctagtcaaaagtgtaccaaacaacgctttacagcaagaacggaatgcgcgtgacgctcgcggtgacgccatttcgccttttcagaaatggataaatagccttgcttcctattatatcttcccaaattaccaatacattacactagcatctgaatttcataaccaatctcgatacaccaaatcg

SEQ ID NO: 71 - DNA, 72 bp, synthetic construct
  misc_feature 1..72: note = Description of Artificial Sequence: Synthetic oligonucleotide
  source 1..72: mol_type = other DNA
atgagttctagctttttatcatccactgcgttcttcctgctcctttgcttaggcttctgccatgtgtcatct

SEQ ID NO: 72 - DNA, 63 bp, synthetic construct
  misc_feature 1..63: note = Description of Artificial Sequence: Synthetic oligonucleotide
  source 1..63: mol_type = other DNA
atggcaaacaagctcttcctcgtctgcgcaactttcgccctctgcttcctcctcaccaacgct

SEQ ID NO: 73 - DNA, 63 bp, synthetic construct
  misc_feature 1..63: note = Description of Artificial Sequence: Synthetic oligonucleotide
  source 1..63: mol_type = other DNA
tcgtacccctacgacgttcctgactacgcccatcatcaccatcaccaccatgatgagttgtag

SEQ ID NO: 74 - DNA, 714 bp, unidentified; identical to SEQ ID NO: 44
  misc_feature 1..714: note = Description of Unknown: Agrobacterium tumefaciens sequence
  source 1..714: mol_type = other DNA

SEQ ID NO: 75 - DNA, 732 bp, synthetic construct
  misc_feature 1..732: note = Description of Artificial Sequence: Synthetic polynucleotide
  source 1..732: mol_type = other DNA
atgggtggtggaggaagtggaggtggaggaagtgtcagtaaaggagaagctgtcataaaggagttcatgaggtttaaggtacacatggaagggtcaatgaacgggcatgaatttgagatcgagggggagggagaaggaaggccttatgagggcactcaaactgctaagttgaaggtcactaagggggggcctcttcctttttcttgggacatactcagccctcagttcatgtatggctcaagggcctttacaaaacaccctgccgatatccccgattactataagcagagctttcccgaaggcttcaagtgggagagggttatgaattttgaagatggcggtgctgttacagtcacacaggacacaagtcttgaggatgggaccctgatttataaagttaagctcagggggacaaatttcccaccagatggaccagtcatgcagaagaaaactatgggatgggaagcctctaccgagcgtctttatcccgaagatggcgttcttaaaggcgatattaagatggctttgaggctcaaagatggcggtcgttacctggccgatttcaaaaccacttacaaagccaaaaaaccagtacagatgccaggcgcttacaacgttgatcgtaagctggacatcacttcccacaacgaggattacacagttgttgaacagtatgagagatcagagggtagacatagtaccggcggaatggatgagctgtacaaaagttcg

SEQ ID NO: 76 - DNA, 263 bp, unidentified
  misc_feature 1..263: note = Description of Unknown: Agrobacterium tumefaciens sequence
  source 1..263: mol_type = other DNA
gtcaagcagatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcga

SEQ ID NO: 77 - DNA, 627 bp, synthetic construct
  misc_feature 1..627: note = Description of Artificial Sequence: Synthetic polynucleotide
  source 1..627: mol_type = other DNA
agagaacttgaagaattgaatgttccaggagagattgttgaaagtctctcctcttctgaggaaagcatcacaagaatcaacaagaaaattgagaagtttcagagtgaagagcaacaacagactgaagatgaattacaagataagattcatccttttgctcaaacacagtcacttgtgtatccattccctggaccaattccaaattctttaccacaaaacattcctcctctgactcaaactcctgtcgttgttcctccgttcttgcaaccagaagttatgggagtttcaaaggttaaagaagcaatggctccaaagcataaagagatgccattcccaaaataccctgtggagcctttcacagaatctcaaagcttgactctcactgatgttgagaatcttcatttgcctcttccattgcttcaatcatggatgcatcaacctcatcagcctttgccaccaacagtgatgtttccacctcaatctgttctctctctttctcagtctaaagttcttccggttccgcagaaagctgtgccttatcctcagagagatatgcctattcaagcttttcttctctaccaagaaccagttttgggtcctgttcgtggtccatttcccatcatagtt

SEQ ID NO: 78 - DNA, 507 bp, synthetic construct
  misc_feature 1..507: note = Description of Artificial Sequence: Synthetic polynucleotide
  source 1..507: mol_type = other DNA
caggagcaaaaccaagagcaacctattcgttgtgaaaaagacgagagattttttagtgataaaattgcaaagtacatcccaatccagtacgttttaagtcgatatccgagctacggtcttaattactatcaacagaagccagttgccttgataaacaaccagtttctcccatatccctactatgctaagccggccgccgtccgttctcctgctcagatattgcagtggcaggttttatccaacactgtcccggcgaagtcttgccaggcgcaacccaccaccatggctagacacccgcatccgcacctttcctttatggctattcctcccaaaaagaaccaagacaagacagagatcccgaccataaatactatcgcttctggtgagccaacttcaacaccgactattgaagcggtggagagcacggtcgccacacttgaagcatccccagaggtgactgaatcacccccggagataaacacggtacaggtcacctcaacggctgtt

SEQ ID NO: 79 - DNA, 507 bp, synthetic construct; identical to SEQ ID NO: 78
  misc_feature 1..507: note = Description of Artificial Sequence: Synthetic polynucleotide
  source 1..507: mol_type = other DNA

SEQ ID NO: 80 - protein, 241 aa, synthetic construct
  REGION 1..241: note = Description of Artificial Sequence: Synthetic polypeptide
MANKLFLVCATFALCFLLTNARPKHPIKHQGLPQEVLNENLLRFFVAPFPEVFGKEKVNELSKDIGSESTEDQAMEDIKQMEAESISSSEEIVPNSVEQKHIQKEDVPSERYLGYLEQLLRLKKYKVPQLEIVPNSAEERLHSMKEGIHAQQKEPMIGVNQELAYFYPELFRQFYQLDAYPSGAWYYVPLGTQYTDAPSFSDIPNPIGSENSEKTTMPLWSSYPYDVPDYAHHHHHHHDEL

SEQ ID NO: 81 - protein, 250 aa, synthetic construct
  REGION 1..250: note = Description of Artificial Sequence: Synthetic polypeptide
MANKLFLVCATFALCFLLTNARELEELNVPGEIVESLSSSEESITRINKKIEKFQSEEQQQTEDELQDKIHPFAQTQSLVYPFPGPIPNSLPQNIPPLTQTPVVVPPFLQPEVMGVSKVKEAMAPKHKEMPFPKYPVEPFTESQSLTLTDVENLHLPLPLLQSWMHQPHQPLPPTVMFPPQSVLSLSQSKVLPVPQKAVPYPQRDMPIQAFLLYQEPVLGPVRGPFPIIVSYPYDVPDYAHHHHHHHDEL

SEQ ID NO: 82 - protein, 210 aa, synthetic construct
  REGION 1..210: note = Description of Artificial Sequence: Synthetic polypeptide
MANKLFLVCATFALCFLLTNAQEQNQEQPIRCEKDERFFSDKIAKYIPIQYVLSRYPSYGLNYYQQKPVALINNQFLPYPYYAKPAAVRSPAQILQWQVLSNTVPAKSCQAQPTTMARHPHPHLSFMAIPPKKNQDKTEIPTINTIASGEPTSTPTIEAVESTVATLEASPEVTESPPEINTVQVTSTAVSYPYDVPDYAHHHHHHHDEL

SEQ ID NO: 83 - protein, 559 aa, synthetic construct
  REGION 1..559: note = Description of Artificial Sequence: Synthetic polypeptide
MLLPKLERSAARPSGEPGCSCAQPAAEAAAPGWAQARGHPGGELEAAASAAGDAGWPNKHTLRILQDFSSDPSSNLTSHSLEKLPPAAEAAEGAPPGQDPGVRRPPDPAHRPLPRDPGPRGPVLPPGLSGDGSLLTRLFQHPLYQVPIPPLTEGDVLFNVNSDIRFNPKAATAENPDWPHEGPEDEFLPTGEAAVDSYPNWLKFHIGINRYELYSRHNPAVGALLQDLGTQKITSVAMKSGGTQLKLIMTFQNYGQALFKPMKQTREQETPPDFFYFSDYERHNAEIAAFHLDRILDFRRVPPVAGRLVNMTKEIRDVTRDKKLWRTFFISPANNVCFYGECSYYCSTEHALCGKPDQIEGSLAAFLPDLALAKRKTWRNPWRRSYHKRKKAEWEVDPDYCEEVRQTPPYDSSHRLLDVMDMTIFDFLMGNMDRHHYETFEKFGNETFIIHLDNGRGFGKHSHDELSILVPLQQCCRIRRSTYLRLQLLAQEEHRLSLLMAEALRADRVAPVLFQPHLEALDRRLRIVLRAVGDCVEKDGLHSVVEDDLGPEHRAAAGR

SEQ ID NO: 84 - protein, 506 aa, synthetic construct
  REGION 1..506: note = Description of Artificial Sequence: Synthetic polypeptide
MHVSSHLGPRVRSRLQPRERPLGCPCARRAASPAPGPAPSAPRRVEPSGGGDPGSKLRALFAHPLYNEPEEPPLLGPEDSLLAGPEALRYYRRKVARWNRRRKMYKEQLNLTSPEPPVQLRQEASWVQFHLGINRHGLYPRSSPVVSKLLQDMRHFPTISADYSQDEKALLGACDCSQIVKPSGVHLKLVLRFSDFGKAMFKPMRQQRDEETPEDFFYFIDFQRHNAEIAAFHLDRILDFRRVPPTVGRLVNVTREILEVTRNEILQSVFFVSPANNVCFFAKCPYMCKTEYAVCGSPHLLEGSLSAFLPSLNLAPRFSMPSPWIRSYSLAGREEWEVNPLYCEAVKQAYPHNSSSRLLNIIDMAIFDFLIGNMDRHHYEMFTKFGEDGFLIHLDNARGFGRHSHDEVSILAPLFQCCRIKRKTLLHLQLLAQADYRLSDVMRESLLEDQLSPVLTEPHLLALDRRLQTVLRTVQDCIEAHGEQSVVADGPVGQWAPDSSRANATS

SEQ ID NO: 85 - protein, 382 aa, Bacillus subtilus
MKVPKTMLLSTAAGLLLSLTATSVSAHYVNEEHHFKVTAHTETDPVASGDDAADDPAIWVHEKHPEKSKLITTNKKSGLVVYDLDGKQLHSYEFGKLNNVDLRYDFPLNGEKIDIAAASNRSEGKNTIEVYAIDGDKGKLKSITDPNHPISTNISEVYGFSLYHSQKTGAFYALVTGKQGEFEQYEIVDGGKGYVTGKKVREFKLNSQTEGLVADDEYGNLYIAEEDEAIWKFNAEPGGGSKGQVVDRATGDHLTADIEGLTIYYAPNGKGYLMASSQGNNSYAMYERQGKNRYVANFEITDGEKIDGTSDTDGIDVLGFGLGPKYPYGIFVAQDGENIDNGQAVNQNFKIVSWEQIAQHLGEMPDLHKQVNPRKLKDRSDG

SEQ ID NO: 86 - protein, 467 aa, Aspergillus niger
MGVSAVLLPLYLLSGVTSGLAVPASRNQSSCDTVDQGYQCFSETSHLWGQYAPFFSLANESVISPEVPAGCRVTFAQVLSRHGARYPTDSKGKKYSALIEEIQQNATTFDGKYAFLKTYNYSLGADDLTPFGEQELVNSGIKFYQRYESLTRNIVPFIRSSGSSRVIASGKKFIEGFQSTKLKDPRAQPGQSSPKIDVVISEASSSNNTLDPGTCTVFEDSELADTVEANFTATFVPSIRQRLENDLSGVTLTDTEVTYLMDMCSFDTISTSTVDTKLSPFCDLFTHDEWINYDYLQSLKKYYGHGAGNPLGPTQGVGYANELIARLTHSPVHDDTSSNHTLDSSPATFPLNSTLYADFSHDNGIISILFALGLYNGTKPLSTTTVENITQTDGFSSAWTVPFASRLYVEMMQCQAEQEPLVRVLVNDRVVPLHGCPVDALGRCTRDSFVRGLSFARSGGDWAECFA

SEQ ID NO: 87 - protein, 479 aa, Aspergillus niger [sequence omitted]

SEQ ID NO: 88 - protein, 532 aa, Arabidopsis thaliana
MTFLLLLLFCFLSPAISSAHSIPSTLDGPFVPVTVPLDTSLRGQAIDLPDTDPRVRRRVIGFEPEQISLSLSSDHDSIWVSWITGEFQIGKKVKPLDPTSINSVVQFGTLRHSLSHEAKGHSLVYSQLYPFDGLLNYTSGIIHHVRITGLKPSTIYYYRCGDPSRRAMSKIHHFRTMPVSSPSSYPGRIAVVGDLGLTYNTTDTISHLIHNSPDLILLIGDVSYANLYLTNGTSSDCYSCSFPETPIHETYQPRWDYWGRFMENLTSKVPLMVIEGNHEIELQAENKTFEAYSSRFAFPFNESGSSSTLYYSFNAGGIHFVMLGAYIAYDKSAEQYEWLKKDLAKVDRSVTPWLVASWHPPWYSSYTAHYREAECMKEAMEELLYSYGTDIVFNGHVHAYERSNRVYNYELDPCGPVYIVIGDGGNREKMAIEHADDPGKCPEPLTTPDPVMGGFCAWNFTPSDKFCWDRQPDYSALRESSFGHGILEMKNETWALWTWYRNQDSSSEVGDQIYIVRQPDRCPLHHRLVNHC

SEQ ID NO: 89 - protein, 512 aa, Glycine max
MWLASFRSLLCKCFIPRWLGLCRLIKTTLIPLERRMLLAMLLNLVLASFVFLSFIRDGSAGITSSFIRSEWPAVDIPLDHEAFAVPKGYNAPQQVHITQGDYDGKAVIISWVTTEEPGHSHIQYGTSENKFQTSEEGTVTNYTFHKYKSGYIHHCLIEGLEYETKYYYRIGSGDSSREFWFKTPPKVDPDSPYKFGIIGDLGQTFNSLSTLEHYIQSGAQTVLFVGDLSYADRYQYNDVGLRWDTWGRFVERSTAYHPWLWSAGNHEIDYMPYMGEVVPFKNYLYRYTTPYLASNSSSPLWYAVRRASAHIIVLSSYSPFVKYTPQYMWLKEELKRVEREKTPWLIVLMHVPLYNSNGAHYMEGESMRSVFESWFIEYKVDVIFAGHVHAYERSYRYSNVDYNITGGNRYPLPNKSAPVYITVGDGGNQEGLASRFLDPQPEYSAFREASYGHSTLEIKNRTHAIYHWNRNDDGKKVPTDSFVLHNQYWGHNRRRRKLKHFLLKVIDEVASM

SEQ ID NO: 90 - protein, 385 aa, Bacillus subtilis
MKKQNDIPQPIRGDKGATVKIPRNIERDRQNPDMLVPPETDHGTVSNMKFSFSDTHNRLEKGGYAREVTVRELPISENLASVNMRLKPGAIRELHWHKEAEWAYMIYGSARVTIVDEKGRSFIDDVGEGDLWYFPSGLPHSIQALEEGAEFLLVFDDGSFSENSTFQLTDWLAHTPKEVIAANFGVTKEEISNLPGKEKYIFENQLPGSLKDDIVEGPNGEVPYPFTYRLLEQEPIESEGGKVYIADSTNFKVSKTIASALVTVEPGAMRELHWHPNTHEWQYYISGKARMTVFASDGHARTFNYQAGDVGYVPFAMGHYVENIGDEPLVFLEIFKDDHYADVSLNQWLAMLPETFVQAHLDLGKDFTDVLSKEKHPVVKKKCSK

SEQ ID NO: 91 - protein, 472 aa, Aspergillus niger
MKPSTLYSSLPWVITSLLTVAVHGAPTGTKSNPPLRGSENLLGYSASNTVTDQSTDEIPYVPVPGQTDAADLGVYLDFEDIENPQPVRGSTGGTDPGPRNDYYDRINSDKLAPPGTDNGQTINAQWPMGLSHNRLGLNESGWARQENEVVMPGATEMAGVDMRLEAGAYRELHWHVASEWSLVLNGSCRIEAVNENGQTFVDDVSAGDVWFFPPGVPHSIQALDSGVEFLLIFDDGSFSEDNTFLATEVFAHQPREVLAKNFDLPVAAFDDIPEDELYIFPGTPAPQNIEEQNVTGSAGVLPKSQSYSYHFSEQPAHEVQGGSVKIVDSLTFPISTNTAAALVTVHPGGMREIHWHPSSDEWTFFISGKARATLFTAPSTATTFDYRPGDVGYFPQSNSHYIENTGDEDLVFLEVLQTEQFSDISLGQWIGSTPKQIVSDTLNLPQSALDRLKTEKMYVVAGSNETDVAATA

SEQ ID NO: 92 - protein, 447 aa, Flammulina velutipes
MFNNFQRLLTVILLSGFTAGVPLASTTTGTGTATGTSTAAEPSATVPFASTDPNPVLWNETSDPALVKPERNQLGATIQGPDNLPIDLQNPDLLAPPTTDHGFVGNAKWPFSFSKQRLQTGGWARQQNEVVLPLATNLACTNMRLEAGAIRELHWHKNAEWAYVLKGSTQISAVDNEGRNYISTVGPGDLWYFPPGIPHSLQATADDPEGSEFILVFDSGAFNDDGTFLLTDWLSHVPMEVILKNFRAKNPAAWSHIPAQQLYIFPSEPPADNQPDPVSPQGTVPLPYSFNFSSVEPTQYSGGTAKIADSTTFNISVAIAVAEVTVEPGALRELHWHPTEDEWTFFISGNARVTIFAAQSVASTFDYQGGDIAYVPASMGHYVENIGNTTLTYLEVFNTDRFADVSLSQWLALTPPSVVQAHLNLDDETLAELKQFATKATVVGPVN

SEQ ID NO: 93 - protein, 514 aa, Arabidopsis thaliana
MDSDTLSGLLENVAKKFPDRRALSVSGKFNLTHARLHDLIERAASRLVSDAGIKPGDVVALTFPNTVEFVIMFLAVIRARATAAPLNAAYTAEEFEFYLSDSDSKLLLTSKEGNAPAQEAASKLKISHVTATLLDAGSDLVLSVADSDSVVDSATELVNHPDDGALFLHTSGTTSRPKGVPLTQLNLASSVKNIKAVYKLTESDSTVIVLPLFHVHGLLAGLLSSLGAGAAVTLPAAGRFSATTFWPDMKKYNATWYTAVPTIHQIILDRHASHPETEYPKLRFIRSCSASLAPVILSRLEEAFGAPVLEAYAMTEATHLMSSNPLPEEGPHKPGSVGKPVGQEMAILNEKGEIQEPNNKGEVCIRGPNVTKGYKNNPEANKAGFEFGWFHTGDIGYFDTDGYLHLVGRIKELINRGGEKISPIEVDAVLLTHPDVSQGVAFGVPDEKYGEEINCAVIPREGTTVTEEDIKAFCKKNLAAFKVPKRVFITDNLPKTASGKIQRRIVAQHFLEKP

SEQ ID NO: 94 - protein, 523 aa, Glycine max
MKDTMETPTTLTTLLRHVAAKFPSRRAISVAAKFDLTHSRLHRLVESAAAQLVSAGVKPGDVVALTFPNTIEFVVMFLAVIRARATAAPLNSAYTAEEFEFYLSDSESKLLLTSPEGNKPAQAAASKLSIPHATASITKAENEEAELSLSLLNHPELNSVNSVESLVNDPDDVALFLHTSGTTSRPKGVPLTQYNLLSSVKNIDSVYRLTESDSTVIVLPLFHVHGLIAGLLSSLGAGAAVALPAAGRFSASAFWKDMIKYSATWYTAVPTIHQIILDRHSSNPEPVYPRLRFIRSCSASLAPVILGKLEEAFGAPVLEAYAMTEASHLMASNPLPQDGAHKSGSVGKPVGQEMGILDESGRVQEAGISGEVCIRGSNVTKGYKNNVAANTASFLFDWFHTGDIGYFDSDGYLHLVGRIKELINRGGEKISGREVDAVLLSHPEIAQAVAFGVPDAKYGEEIYCAVIPREGSNVDEAEVLRFSKTNLASFKVPKKVFITDSLPKTATGKILRRLVAEHFVSQI

SEQ ID NO: 95 - protein, 523 aa, Glycine max
MKDTMETPTTLTTLLRHVAAKFPSRRAISVAAKFDLTHSRLHRLVESAAAQLVSAGVKPGDVVALTFPNTIEFVVMFLAVIRARATAAPLNSAYTAEEFEFYLSDSESKLLLTSPEGNKPAQAAASKLSIPHATASITKAENEEAELSLSLLNHPELNSVNSVESLVNDPDDVALFLHTSGTTSRPKGVPLTQYNLLSSVKNIDSVYRLTESDSTVIVLPLFHVHGLIAGLLSSLGAGAAVALPAAGRFSASAFWKDMIKYSATWYTAVPTIHQIILDRHSSNPEPVYPRLRFIRSCSASLAPVILGKLEEAFGAPVLEAYAMTEASHLMASNPLPQDGAHKSGSVGKPVGQEMGILDESGRVQEAGISGEVCIRGSNVTKGYKNNVAANTASFLFDWFHTGDIGYFDSDGYLHLVGRIKELINRGGEKISPIEVDAVLLSHPEIAQAVAFGVPDAKYGEEIYCAVIPREGSNVDEAEVLRFSKTNLASFKVPKKVFITDSLPKTATGKILRRLVAEHFVSQI

SEQ ID NO: 96 - DNA, 1533 bp, Glycine max
  source 1..1533: mol_type = other DNA
atgttcatcgagaattttaaggttgagtgtcctaatgtgaagtacaccgagactgagattcagtccgtgtacaactacgaaaccaccgaacttgttcacgagaacaggaatggcacctatcagtggattgtcaaacccaaatctgtcaaatacgaatttaaaaccaacatccatgttcctaaattaggggtaatgcttgtgggttggggtggaaacaacggctcaaccctcaccggtggtgttattgctaaccgagagggcatttcatgggctacaaaggacaagattcaacaagccaattactttggctccctcacccaagcctcagctatccgagttgggtccttccagggagaggaaatctatgccccattcaagagcctgcttccaatggttaaccctgacgacattgtgtttgggggatgggatatcagcaacatgaacctggctgatgccatggccagggcaaaggtgtttgacatcgatttgcagaagcagttgaggccttacatggaatccatgcttccactccccggaatctatgacccggatttcattgctgccaaccaagaggagcgtgccaacaacgtcatcaagggcacaaagcaagagcaagttcaacaaatcatcaaagacatcaaggcgtttaaggaagccaccaaagtggacaaggtggttgtactgtggactgccaacacagagaggtacagtaatttggttgtgggccttaatgacaccatggagaatctcttggctgctgtggacagaaatgaggctgagatttctccttccaccttgtatgccattgcttgtgttatggaaaatgttcctttcattaatggaagccctcagaacacttttgtaccagggctgattgatcttgccatcgcgaggaacactttgattggtggagatgacttcaagagtggtcagaccaaaatgaaatctgtgttggttgatttccttgtgggggctggtatcaagccaacatctatagtcagttacaaccatctgggaaacaatgatggtatgaatctttcggctccacaaactttccgttccaaggaaatctccaagagcaacgttgttgatgatatggtcaacagcaatgccatcctctatgagcctggtgaacatccagaccatgttgttgttattaagtatgtgccttacgtaggggacagcaagagagccatggatgagtacacttcagagatattcatgggtggaaagagcaccattgttttgcacaacacatgcgaggattccctcttagctgctcctattatcttggacttggtccttcttgctgagctcagcactagaatcgagtttaaagctgaaaatgagggaaaattccactcattccacccagttgctaccatcctcagctacctcaccaaggctcctctggttccaccgggtacaccagtggtgaatgcattgtcaaagcagcgtgcaatgctggaaaacataatgagggcttgtgttggattggccccagagaataacatgattctcgagtacaagtga

(19) United States
(12) Patent Application Publication
(10) Pub. No.: US 2025/0354161 A1
(43) Pub. Date: Nov. 20, 2025

(54) RECOMBINANT MICELLE AND METHOD OF IN VIVO ASSEMBLY

(71) Applicant: Mozza Foods, Inc., Los Angeles, CA (US)

(72) Inventors: Cory J. Tobin, Los Angeles, CA (US); Noah BROOKES, Los Angeles, CA (US)

(73) Assignee: Mozza Foods, Inc., Los Angeles, CA (US)

(21) Appl. No.: 19/224,244

(22) Filed: May 30, 2025

Related U.S. Application Data
(63) Continuation of application No. 17/826,021, filed on May 26, 2022, now Pat. No. 12,359,212.
Application No. 17/826,021 is a continuation-in-part of application No. 17/717,000, filed on Apr. 8, 2022, now Pat. No. 11,718,856.
Application No. 17/717,000 is a continuation of application No. 16/741,680, filed on Jan. 13, 2020, now Pat. No. 11,326,176.
(60) Provisional application No. 62/939,247, filed on Nov. 22, 2019.
(60) Provisional application No. 63/281,069, filed on Nov. 18, 2021.

(51) Int. Cl. C12N 15/82; B01D 61/14

(52) U.S. Cl. CPC C12N 15/8202; B01D 61/145; B01D 61/146; B01D 61/147; C12N 15/8218; B01D 2315/16

(57) ABSTRACT

A plant cell co-expressing at least one casein protein and at least one kinase. The at least one casein protein is phosphorylated by the at least one kinase in vivo. Casein micelles comprising phosphorylated κ-casein and at least one of αS1-casein, αS2-casein, and β-casein can be made in vivo and/or in vitro. The casein micelles can be used to make food products including milk and cheese.

### CROSS REFERENCE TO RELATED APPLICATION(S)

This application is a continuation of U.S. patent application Ser. No. 17/826,021, filed on May 26, 2022, which is a continuation-in-part (CIP) of U.S. patent application Ser. No. 17/717,000 filed on Apr. 8, 2022, now patented as U.S. Pat. No. 11,718,856, issued on Aug. 8, 2023, claims the benefit of U.S. Provisional Patent Application No. 63/281,069, filed on Nov. 18, 2021, is also a continuation of U.S. patent application Ser. No. 16/741,680, filed on Jan. 13, 2020, now patented as U.S. Pat. No. 11,326,176, issued on May 10, 2022, which claims the benefit of U.S. Provisional Patent Application No. 62/939,247, filed on Nov. 22, 2019, all of which are incorporated herein by reference in their entireties.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### SEQUENCE LISTING

The present application is being filed along with a Sequence Listing in electronic format. The Sequence Listing is provided as a file entitled SEQLIST_701002003USCON.xml, created on May 29, 2025, which is 161 kilobytes in size. The information in the electronic format of the Sequence Listing is incorporated by reference in its entirety.

### TECHNICAL FIELD

An embodiment of the present disclosure relates generally to a micelle and more particularly to recombinant micelle and method of in vivo assembly in a plant cell.

### BACKGROUND

Casein micelles account for more than 80% of the protein in bovine milk and are a key component of all dairy cheeses. Casein micelles include individual casein proteins are produced in the mammary glands of bovines and other ruminants. The industrial scale production of the milk that is processed to yield these casein micelles, primarily in the form of curds for cheese production, typically occurs on large-scale dairy farms and is often inefficient, damaging to the environment, and harmful to the animals. Dairy cows contribute substantially to greenhouse gasses, consume significantly more water than the milk they produce, and commonly suffer from dehorning, disbudding, mastitis, routine forced insemination, and bobby calf slaughter.

Accordingly, there is a need for an in vivo plant-based casein expression system which allows for purification of biologically active casein proteins that is cost effective at industrial scale.

Protein phosphorylation is a post-translational modification of proteins in which a phosphate group is added to an amino acid in the protein. Chemical phosphorylation of food proteins can be achieved by using chemicals. However, chemical phosphorylation disrupts the native structure of food proteins because of the harsh reaction conditions. Moreover, unwanted chemical reagents from the final product can be difficult to remove. Enzymatic phosphorylation with ATP is a more desirable method to phosphorylate food proteins due to improved food safety. However, this method does not fit the needs of industrial-scale production due to the high cost of ATP and enzymes.

Solutions to these problems have been long sought but prior developments have not taught or suggested any solutions and, thus, solutions to these problems have long eluded those skilled in the art.

### DISCLOSURE OF INVENTION

An embodiment of the present invention provides a method of in vivo assembly of a recombinant micelle including: introducing a plasmid into a plant cell, wherein: the plasmid includes a segment of deoxyribonucleic acid (DNA) for encoding a ribonucleic acid (RNA) for a protein in a casein micelle, the segment of DNA is transcribed and translated; forming recombinant casein proteins in the plant cell, wherein: the recombinant casein proteins include a κ-casein and at least one of an αS₁-casein, an αS₂-casein, a β-casein; and assembling in vivo a recombinant micelle within the plant cell, wherein: an outer layer of the recombinant micelle is enriched with the κ-casein, an inner matrix of the recombinant micelle include at least one of the αS₁-casein, the αS₂-casein, the β-casein.

An embodiment of the present invention provides a recombinant micelle including: an outer layer enriched with a κ-casein; and an inner matrix including at least one of a αS₁-casein, a αS₂-casein, a β-casein.

An embodiment of the present invention provides a plasmid including a segment of deoxyribonucleic acid (DNA) for encoding a protein in a casein micelle wherein the segment of DNA includes a promoter and a N-terminal signal peptide.

Certain embodiments of the disclosure have other steps or elements in addition to or in place of those mentioned above. The steps or elements will become apparent to those skilled in the art from a reading of the following detailed description when taken with reference to the accompanying drawings.

Some aspects of the present disclosure provide methods of in vivo assembly of a recombinant micelle comprising introducing a plasmid into a plant cell, wherein the plasmid includes a segment of deoxyribonucleic acid (DNA) for encoding a ribonucleic acid (RNA) for a protein in a casein micelle, the segment of DNA is transcribed and translated; forming recombinant casein proteins in the plant cell, wherein the recombinant casein proteins include a κ-casein and at least one of an αS1-casein, an αS₂-casein, and a β-casein; and assembling in vivo a recombinant micelle within the plant cell, wherein an outer layer of the recombinant micelle is enriched with the κ-casein and an inner matrix of the recombinant micelle include at least one of the αS1-casein, the αS2-casein, the β-casein.

In some cases, the plasmid includes a further segment of DNA encoding a N-terminal signal peptide that targets the recombinant casein proteins to a vacuole in the plant cell. In some cases, the plasmid includes a further segment of DNA encoding a selectable marker or a screenable marker. In some cases, the plasmid includes a further segment of DNA encoding interference RNA to suppress expression of a native protein or a native peptide in the plant cell. In some cases, the plasmid includes a further segment of DNA encoding a protein capable of altering an intracellular environment of the plant cell.

In some cases, the disclosed method further comprises introducing a further plasmid into the plant cell; wherein the further plasmid includes a further segment of DNA for encoding a further RNA for a further protein in the casein micelle; the further segment of DNA is transcribed and translated; and the further segment of DNA is at least one of the encoding a N-terminal signal peptide that targets the recombinant casein proteins to an endoplasmic reticulum in the plant cell, a further N-terminal signal peptide that targets the recombinant casein proteins to a vacuole in the plant cell, a selectable marker or a screenable marker, and a protein capable of altering an intracellular environment of the plant cell. In some cases, the plasmid includes a further segment of DNA including one or more nucleotide sequences selected from SEQ ID NO:36 to SEQ ID NO:43.

Some aspects of the present disclosure provides a recombinant micelle comprising an outer layer enriched with a κ-casein; and an inner matrix including at least one of a αS1-casein, a αS2-casein, a β-casein. In some cases, the inner matrix includes a calcium and a phosphate.

Some aspects of the present disclosure provide plasmids comprising a segment of deoxyribonucleic acid (DNA) for encoding a protein in a casein micelle wherein the segment of DNA includes a promoter and a N-terminal signal peptide. In some cases, the plasmid includes a further segment of DNA encoding a N-terminal signal peptide that targets the recombinant casein proteins to a vacuole in a plant cell. In some cases, the plasmid includes a further segment of DNA encoding a selectable marker or a screenable marker. In some cases, the plasmid includes a further segment of DNA encoding interference RNA to suppress expression of a native protein or a native peptide in a plant cell. In some cases, the plasmid includes the plasmid includes a further segment of DNA encoding a protein capable of altering an intracellular environment of a plant cell. In some cases, the plasmid includes a further segment of DNA including one or more nucleotide sequences selected from SEQ ID NO:36 to SEQ ID NO:43.

Some aspects of the present disclosure provide methods of isolating a recombinant micelle comprising processing a seed including a cytoplasm with the recombinant micelle; microfiltering the cytoplasm to remove a particulate above 2 um; ultrafiltering the cytoplasm microfiltered to a further particulate greater than 100 nm; and collecting the recombinant micelle from the cytoplasm ultrafiltered. In some cases, the disclosed methods further comprise processing the seed includes cleaning, and deshelling or dehulling the seed, flaking the seed cleaned to 0.005-0.02 inch thickness, extracting with a solvent of oil from the seed flaked, desolventizing the seed flaked without cooking and collecting the de-oiled, cleaned separating the recombinant micelle into a slurry by hydrating, agitating and wet milling the seed flaked, passing the slurry through a mesh screen to remove a particulate above 0.5 mm in size and collecting a permeate; and microfiltering the cytoplasm includes microfiltering the permeate.

In some cases, the disclosed methods further comprise microfiltering the cytoplasm includes microfiltering a permeate; ultrafiltering the cytoplasm microfiltered includes ultrafiltering the permeate microfiltered; and collecting the recombinant micelle from the cytoplasm ultrafiltered includes collecting a retentate from the permeate ultrafiltered.

In some cases, the disclosed methods further comprise microfiltering the cytoplasm includes microfiltering a permeate; ultrafiltering the cytoplasm microfiltered includes ultrafiltering the permeate microfiltered; collecting the recombinant micelle from the cytoplasm ultrafiltered includes collecting a retentate from the permeate ultrafiltered; and diafiltering the retentate at a rate that the permeate is collected and passing the retentate through the ultrafiltering. In some cases, the disclosed methods further comprise processing the seed milled from a maize, a rice, a sorghum, a cowpea, a soybean, a cassava, a coyam, a sesame, a peanut, a pea, a cotton, a yam, or a combination thereof.

The current disclosure provides compositions, methods and systems for phosphorylation of proteins in plants. Described herein, in some aspects, are vectors for expressing a phosphorylated payload protein in a plant, wherein a vector may comprise at least one of a polynucleotide sequence encoding: a first kinase, a second kinase, a first payload protein, a promoter sequence, a terminator sequence, a second payload protein, and combinations thereof. In some instances, described herein are vectors for expressing a phosphorylated payload protein in a plant, wherein a vector may comprise, for example, a polynucleotide sequence encoding: a first kinase, a second kinase, a first payload protein, a promoter sequence, a terminator sequence, and optionally a second payload protein.

Contemplated promoters include CaMV 35S, AtuMas Pro+5′UTR, RbcS2 promoter, a soybean GY1 Promoter, soybean CG1 Promoter, or other suitable promoters.

Contemplated terminator sequence can be octopine synthase terminator (Ocst), Octopine (OCS) terminator, NOS terminator or other suitable terminator sequences. It is contemplated that the first or the second kinase can be a human kinase or a non-human kinase, for example, a bovine kinase. In some instances, at least one of the first and the second kinase is FAM20A, FAM20C, casein Kinase II or a tyrosine kinase. In some instances, at least one of the first kinase and the second kinase has at least 50%, 60%, 70%, 80%, 85%, 90%, 95%, or 99% sequence identity to SEQ ID NO: 40, SEQ ID NO: 41, SEQ ID NO: 43, SEQ ID NO: 83, or SEQ ID NO: 84. In some instances, the first kinase is different from the second kinase. For example, the first kinase can any one of the kinases mentioned herein, and the second kinase can be a different kinase mentioned herein.

In some instances, the first or second payload (e.g., casein) protein is a mammalian protein, for example, a human protein, a ruminant protein, a primate protein. In some instances, the ruminant animal includes, for example, a cow, a buffalo, a yak, a deer, a bovine, a goat, and a sheep. In some instances, the first or second payload protein comprises a whey protein, including, for example, α-lactalbumin, β-lactoglobulin, serum albumin, immunoglobulins, and proteose peptone. In some instances, the payload protein comprises an egg white protein, including, for example, ovalbumin, ovotransferrin, ovomucoid, ovoglobulin g2, ovoglobulin g3, ovomucin, lysozyme, ovoinhibitor, ovoglycoprotein, flavoprotein, ovomacroglobulin, avidin, and cystatin. In some instances, the egg white protein has at least 50%, 60%, 70%, 80%, 85%, 90%, 95%, or 99% sequence identity to the amino acid SEQ ID NO: 80, SEQ ID NO: 81, SEQ ID NO: 82, SEQ ID NO: 83, SEQ ID NO: 86, SEQ ID NO: 87, SEQ ID NO: 88, SEQ ID NO: 90, SEQ ID NO: 91, or SEQ ID NO: 92.

In some instances, the payload protein is a collagen protein, including, for example, Collagen I, Collagen II, Collagen III, Collagen IV, Collagen V, Collagen VI, Collagen VII, Collagen VIII, Collagen IX, Collagen X, Collagen XI, Collagen XII, Collagen XIII, Collagen XIV, Collagen XV, Collagen XVI, Collagen XVII, Collagen XVIII, Collagen XIX, Collagen XX, Collagen XXI, Collagen XXII, Collagen XXIII, Collagen XXIV, Collagen XXV, Collagen XXVI, Collagen XXVII, and Collagen XXVIII. In some instances, the collagen protein comprises one or more a chains, for example, wild type Bovine Collagen Alpha-1(I) Chain. In some instances, the collagen protein expressed has at least 50%, 60%, 70%, 80%, 85%, 90%, 95%, or 99% sequence identity to the amino acid SEQ ID NO: 49.

In some instances, the first or second payload protein is a casein protein, including, for example, αS1-casein, αS2-casein, β-casein, and κ-casein. The casein protein can be from any mammalian species (including human) including from a ruminant animal. In some instances, the casein protein has at least 50%, 60%, 70%, 80%, 85%, 90%, 95%, or 99% sequence identity to the amino acid SEQ ID NO:36, SEQ ID NO:37, SEQ ID NO:38, SEQ ID NO:39, SEQ ID NO:77, SEQ ID NO:78, SEQ ID NO:79, SEQ ID NO:80, SEQ ID NO:81, or SEQ ID NO: 82. In some instances, the second payload protein is different from the first payload protein. For example, the first payload protein is κ-casein, and the second payload protein is at least one of αS1-casein, αS2-casein, and β-casein. It is contemplated that the same vector can express casein proteins from different species, for example, the first pay load protein is human κ-casein, and second pay load protein is a bovine αS1-casein, αS2-casein, or β-casein. As another example, the first pay load protein κ-casein is a bovine casein, and second pay load protein is a human β-casein.

TABLE 1
Examples of certain proteins used in the current
disclosure and their sequences.

| | | |
|---|---|---|
| SEQ | Wild type | MMKSFFLVVTILALTLPFLGAQEQN |
| ID NO: | Bovine κ | QEQPIRCEKDERFFSDKIAKYIPIQ |
| 36 | casein | YVLSRYPSYGLNYYQQKPVALINNO |
| | | FLPYPYYAKPAAVRSPAQILQWQVL |
| | | SNTVPAKSCQAQPTTMARHPHPHLS |
| | | FMAIPPKKNQDKTEIPTINTIASGE |
| | | PTSTPTTEAVESTVATLEDSPEVIE |
| | | SPPEINTVQVTSTAV |
| SEQ | Wild type | MKLLILTCLVAVALARPKHPIKHQG |
| ID | Bovine αs1- | LPQEVLNENLLRFFVAPFPEVFGKE |
| NO: 37 | casein | KVNELSKDIGSESTEDQAMEDIKQM |
| | | EAESISSSEEIVPNSVEQKHIQKED |
| | | VPSERYLGYLEQLLRLKKYKVPOLE |
| | | IVPNSAEERLHSMKEGIHAQQKEPM |
| | | IGVNQELAYFYPELFRQFYQLDAYP |
| | | SGAWYYVPLGTQYTDAPSFSDIPNP |
| | | IGSENSEKTTMPLW |
| SEQ | Wild type | MKFFIFTCLLAVALAKNTMEHVSSS |
| ID | Bovine αS1- | EESIISQETYKQEKNMAINPSKENL |
| NO: 38 | casein | CSTFCKEVVRNANEEEYSIGSSSEE |
| | | SAEVATEEVKITVDDKHYQKALNEI |
| | | NQFYQKFPQYLQYLYQGPIVLNPWD |
| | | QVKRNAVPITPTLNREQLSTSEENS |
| | | KKTVDMESTEVFTKKTKLTEEEKNR |
| | | LNFLKKISQRYQKFALPQYLKTVYQ |
| | | HQKAMKPWIQPKTKVIPYVRYL |
| SEQ | Wild type | MKVLILACLVALALARELEELNVPG |
| ID | Bovine β- | EIVESLSSSEESITRINKKIEKFQS |
| NO: 39 | casein | EEQQQTEDELQDKIHPFAQTQSLVY |
| | | PFPGPIPNSLPQNIPPLTQTPVVVP |
| | | PFLQPEVMGVSKVKEAMAPKHKEMP |
| | | FPKYPVEPFTESQSLTLTDVENLHL |
| | | PLPLLQSWMHQPHOPLPPTVMFPPQ |
| | | SVLSLSQSKVLPVPOKAVPYPQRDM |
| | | PIQAFLLYQEPVLGPVRGPFPIIV |
| SEQ | FAM20A | MSSSFLSSTAFFLLLCLGFCHVSSO |
| ID | | LRPRERPRGCPCTGRASSLARDSAA |
| NO: 40 | | AASDPGTIVHNFSRTEPRTEPAGGS |
| | | HSGSSSKLQALFAHPLYNVPEEPPL |
| | | LGAEDSLLASQEALRYYRRKVARWN |
| | | RRHKMYREQMNLTSLDPPLQLRLEA |
| | | SWVQFHLGINRHGLYSRSSPVVSKL |
| | | LQDMRHFPTISADYSQDEKALLGAC |
| | | DCTQIVKPSGVHLKLVLRFSDFGKA |
| | | MFKPMRQQRDEETPVDFFYFIDFOR |
| | | HNAEIAAFHLDRILDFRRVPPTVGR |
| | | IVNVTKEILEVTKNEILQSVFFVSP |
| | | ASNVCFFAKCPYMCKTEYAVCGNPH |
| | | LLEGSLSAFLPSLNLAPRLSVPNPW |
| | | IRSYTLAGKEEWEVNPLYCDTVKQI |
| | | YPYNNSQRLLNVIDMAIFDFLIGNM |
| | | DRHHYEMFTKFGDDGFLIHLDNARG |
| | | FGRHSHDEISILSPLSQCCMIKKKT |
| | | LLHLQLLAQADYRLSDVMRESLLED |
| | | QLSPVLTEPHLLALDRRLQTILRTV |
| | | EGCIVAHGQQSVIVDGPVEQLAPDS |
| | | GQANLTSHDEL |
| SEQ | FAM20C | MSSSFLSSTAFFLLLCLGFCHVSSL |
| ID | | DLLPRLERRGARPSGEPGCSCAQPA |
| NO: 41 | | AEVAAPGWAQVRGRPGEPPAASSAA |
| | | GDAGWPNKHTLRILQDFSSDPSSNL |
| | | SSHSLEKLPPAAEPAERALRGRDPG |
| | | ALRPHDPAHRPLLRDPGPRRSESPP |
| | | GPGGDASLLARLFEHPLYRVAVPPL |
| | | TEEDVLFNVNSDTRLSPKAAENPDW |
| | | PHAGAEGAEFLSPGEAAVDSYPNWL |
| | | KFHIGINRYELYSRHNPAIEALLHD |
| | | LSSQRITSVAMKSGGTQLKLIMTFO |
| | | NYGQALFKPMKQTREQETPPDFFYF |
| | | SDYERHNAEIAAFHLDRILDFRRVP |
| | | PVAGRMVNMTKEIRDVTRDKKLWRT |
| | | FFISPANNICFYGECSYYCSTEHAL |
| | | CGKPDQIEGSLAAFLPDLSLAKRKT |
| | | WRNPWRRSYHKRKKAEWEVDPDYCE |
| | | EVKQTPPYDSSHRILDVMDMTIFDF |
| | | LMGNMDRHHYETFEKFGNETFIIHL |
| | | DNGRGFGKYSHDELSILVPLQQCCR |
| | | IRKSTYLRLQLLAKEEYKLSLLMAE |
| | | SLRGDQVAPVLYQPHLEALDRRLRV |
| | | VLKAVRDCVERNGLHSVVDDDLDTE |
| | | HRAASARHDEL |
| SEQ | CaMV 35S | GTCAACATGGTGGAGCACGACACTC |
| ID | Promoter | TGGTCTACTCCAAAAATGTCAAAGA |
| NO: 42 | | TACAGTCTCAGAAGATCAAAGGGCT |
| | | ATTGAGACTTTTCAACAAAGGATAA |
| | | TTTCGGGAAACCTCCTCGGATTCCA |
| | | TTGCCCAGCTATCTGTCACTTCATC |
| | | GAAAGGACAGTAGAAAAGGAAGGTG |
| | | GCTCCTACAAATGCCATCATTGCGA |
| | | TAAAGGAAAGGCTATCATTCAAGAT |
| | | CTCTCTGCCGACAGTGGTCCCAAAG |
| | | ATGGACCCCCACCCACGAGGAGCAT |
| | | CGTGGAAAAAGAAGAGGTTCCAACC |
| | | ACGTCTACAAAGCAAGTGGATTGAT |
| | | GTGACATCTCCACTGACGTAAGGGA |
| | | TGACGCACAATCCCACTATCCTTCG |
| | | CAAGACCCTTCCTCTATATAAGGAA |
| | | GTTCATTTCATTTGGAGAGGACACG |
| | | C |
| SEQ | Wild Type | MSGPVPSRARVYTDVNTHRPREYWD |
| ID | Human | YESHVVEWGNQDDYQLVRKLGRGKY |
| NO: 43 | Casein | SEVFEAINITNNEKVVVKILKPVKK |
| | Kinase II | KKIKREIKILENLRGGPNIITLADI |
| | | VKDPVSRTPALVFEHVNNTDFKQLY |
| | | QTLTDYDIRFYMYEILKALDYCHSM |
| | | GIMHRDVKPHNVMIDHEHRKLRLID |
| | | WGLAEFYHPGQEYNVRVASRYFKGP |
| | | ELLVDYQMYDYSLDMWSLGCMLASM |
| | | IFRKEPFFHGHDNYDQLVRIAKVLG |
| | | TEDLYDYIDKYNIELDPRFNDILGR |
| | | HSRKRWERFVHSENQHLVSPEALDF |
| | | LDKLLRYDHQSRLTAREAMEHPYFY |
| | | TVVKDQARMGSSSMPGGSTPVSSAN |
| | | MMSGISSVPTPSPLGPLAGSPVIAA |
| | | ANPLGMPVPAAAGAQQ |
| SEQ | octopine | GTCCTGCTTTAATGAGATATGCGAG |
| ID | synthase | AAGCCTATGATCGCATGATATTTGC |
| NO: 44 | terminator | TTTCAATTCTGTTGTGCACGTTGTA |
| | | AAAAACCTGAGCATGTGTAGCTCAG |
| | | ATCCTTACCGCCGGTTTCGGTTCAT |
| | | TCTAATGAATATATCACCCGTTACT |
| | | ATCGTATTTTTATGAATAATATTCT |
| | | CCGTTCAATTTACTGATTGTACCCT |
| | | ACTACTTATATGTACAATATTAAAA |
| | | TGAAAACAATATATTGTGCTGAATA |
| | | GGTTTATAGCGACATCTATGATAGA |
| | | GCGCCACAATAACAAACAATTGCGT |
| | | TTTATTATTACAAATCCAATTTTAA |
| | | AAAAAGCGGCAGAACCGGTCAAACC |
| | | TAAAAGACTGATTACATAAATCTTA |
| | | TTCAAATTTCAAAAGTGCCCCAGGG |
| | | GCTAGTATCTACGACACACCGAGCG |
| | | GCGAACTAATAACGCTCACTGAAGG |
| | | GAACTCCGGTTCCCCGCCGGCGCGC |
| | | ATGGGTGAGATTCCTTGAAGTTGAG |
| | | TATTGGCCGTCCGCTCTACCGAAAG |
| | | TTACGGGCACCATTCAACCCGGTCC |
| | | AGCACGGCGGCCGGGTAACCGACTT |
| | | GCTGCCCCGTGCAGGTCAAACCTTG |
| | | ACAGTGACGACAAATCGTTGGGCGG |
| | | GTCCAGGGCGAATTTTGCGACAACA |
| | | TGTCGAGGCTCAGCAGGAC |
| SEQ | Ovalbumin | MGSIGAASMEFCFDVFKELKVHHAN |
| ID | | ENIFYCPIAIMSALAMVYLGAKDST |
| NO: 45 | | RTQINKVVRFDKLPGFGDSIEAQCG |
| | | TSVNVHSSLRDILNQITKPNDVYSF |
| | | SLASRLYAEERYPILPEYLQCVKEL |
| | | YRGGLEPINFQTAADQARELINSWV |
| | | ESQTNGIIRNVLOPSSVDSQTAMVL |
| | | VNAIVFKGLWEKAFKDEDTQAMPFR |
| | | VTEQESKPVQMMYQIGLFRVASMAS |
| | | EKMKILELPFASGTMSMLVLLPDEV |
| | | SGLEQLESIINFEKLTEWTSSNVME |
| | | ERKIKVYLPRMKMEEKYNLTSVLMA |
| | | MGITDVFSSSANLSGISSAESLKIS |
| | | QAVHAAHAEINEAGREVVGSAEAGV |
| | | DAASVSEEFRADHPFLFCIKHIATN |
| | | AVLFFGRCVSP |
| SEQ | Ovotransferrin | MKLILCTVLSLGIAAVCFAAPPKSV |
| ID | | IRWCTISSPEEKKCNNLRDLTQQER |
| NO: 46 | | ISLTCVQRATYLDCTKAIANNEADA |
| | | ISLDGGQAFEAGLAPYKLKPIAAEV |
| | | YEHTEGSTTSYYAVAVVKKGTEFTV |
| | | NDLQGKTSCHTGLGRSAGWNIPIGT |
| | | LLHRGAIEWEGIESGSVEQAVARFF |
| | | SASCVPGATIEQKLCRQCKGDPKTK |
| | | CARNAPYSGYSGAFHCLKDGKGDVA |
| | | FVKHTTVNENAPDQKDEYELLCLDG |
| | | SRQPVDNYKTCNWARVAAHAVVARD |
| | | DNKVEDIWSFLSKAQSDFGVDTKSD |
| | | FHLFGPPGKKDPVLKDLLFKDSAIM |
| | | LKRVPSLMDSQLYLGFEYYSAIQSM |
| | | RKDQLTPSPRENRIQWCAVGKDEKS |
| | | KCDRWSVVSNGDVECTVVDETKDCI |
| | | IKIMKGEADAVALDGGLVYTAGVCG |
| | | LVPVMAERYDDESQCSKTDERPASY |
| | | FAVAVARKDSNYNWNNLKGKKSCHT |
| | | AVGRTAGWVIPMGLIHNRTGTCNFD |
| | | EYFSEGCAPGSPPNSRLCQLCQGSG |
| | | GIPPEKCVASSHEKYFGYTGALRCL |
| | | VEKGDVAFIQHSTVEENTGGKNKAD |
| | | WAKNLQMDDFELLCTDGRRANVMDY |
| | | RECNLAEVPTHAVVVRPEKANKIRD |
| | | LLERQEKRFGVNGSEKSKFMMFESO |
| | | NKDLLFKDLTKCLFKVREGTTYKEF |
| | | LGDKFYTVISSLKTCNPSDILQMCS |
| | | FLEGK |
| SEQ | Ovomucoid | MAMAGVFVLFSFVLCGFLPDAAFGA |
| ID | | EVDCSRFPNATDKEGKDVLVCNKDL |
| NO: 47 | | RPICGTDGVTYTNDCLLCAYSIEFG |
| | | TNISKEHDGECKETVPMNCSSYANT |
| | | TSEDGKVMVLCNRAFNPVCGTDGVT |
| | | YDNECLLCAHKVEQGASVDKRHDGG |
| | | CRKELAAVSVDCSEYPKPDCTAEDR |
| | | PLCGSDNKTYGNKCNFCNAVVESNG |
| | | TLTLSHFGKC |
| SEQ | Ovoglobulin | MGALLALLDPVQPTRAPDCGGILTP |
| ID | G2 | LGLSYLAEVSKPHAEVVLRQDLMAQ |
| NO: 48 | | RASDLFLGSMEPSRNRITSVKVADL |
| | | WLSVIPEAGLRLGIEVELRVAPLHA |
| | | VPMPVRISIRADLHVDMGPDGNLQL |
| | | LTSACRPTVQAQSTREAESKSSRSI |
| | | LDKVVDVDKLCLDVSKLLLFPNEQL |
| | | MSLTALFPVTPNCQLQYLPLAAPVF |
| | | SKQGIALSLOTTFQVAGAVVPVPVS |
| | | PVPFSMPELASTSTSHLILALSEHF |
| | | YTSLYFTLERAGAFNMTI |
| | | PSMLTTATLAQKITQVGSLYHEDLP |
| | | ITLSAALRSSPRVVLEEGRAALKLF |
| | | LTVHIGAGSPDFQSFLSVSADVTAG |
| | | LQLSVSDTRMMISTAVIEDAELSLA |
| | | ASNVGLVRAALLEELFLAPVCQQVP |
| | | AWMDDVLREGVHLPHLSHFTYTDVS |
| | | VVVHKDYVLVPCKLKLRSTMA |
| SEQ | Wild type | MFSFVDLRLLLLLAATALLTHGQEE |
| ID | Bovine | GQEEGQEEDIPPVTCVQNGLRYHDR |
| NO: 49 | Collagen | DVWKPVPCQICVCDNGNVLCDDVIC |
| | Alpha-1(I) | DELKDCPNAKVPTDECCPVCPEGQE |
| | Chain | SPTDQETTGVEGPKGDTGPRGPRGP |
| | | AGPPGRDGIPGQPGLPGPPGPPGPP |
| | | GPPGLGGNFAPQLSYGYDEKSTGIS |
| | | VPGPMGPSGPRGLPGPPGAPGPQGF |
| | | QGPPGEPGEPGASGPMGPRGPPGPP |
| | | GKNGDDGEAGKPGRPGERGPPGPQG |
| | | ARGLPGTAGLPGMKGHRGFSGLDGA |
| | | KGDAGPAGPKGEPGSPGENGAPGQM |
| | | GPRGLPGERGRPGAPGPAGARGNDG |
| | | ATGAAGPPGPTGPAGPPGFPGAVGA |
| | | KGEGGPQGPRGSEGPQGVRGEPGPP |
| | | GPAGAAGPAGNPGADGQPGAKGANG |
| | | APGIAGAPGFPGARGPSGPQGPSGP |
| | | PGPKGNSGEPGAPGSKGDTGAKGEP |
| | | GPTGIQGPPGPAGEEGKRGARGEPG |
| | | PAGLPGPPGERGGPGSRGFPGADGV |
| | | AGPKGPA |
| | | GERGAPGPAGPKGSPGEAGRPGEAGL |
| | | PGAKGLTGSPGSPGPDGKTGPPGPA |
| | | GQDGRPGPPGPPGARGQAGVMGFPG |
| | | PKGAAGEPGKAGERGVPGPPGAVGP |
| | | AGKDGEAGAQGPPGPAGPAGERGEQ |
| | | GPAGSPGFQGLPGPAGPPGEAGKPG |
| | | EQGVPGDLGAPGPSGARGERGFPGE |
| | | RGVQGPPGPAGPRGANGAPGNDGAK |
| | | GDAGAPGAPGSQGAPGLQGMPGERG |
| | | AAGLPGPKGDRGDAGPKGADGAPGK |
| | | DGVRGLTGPIGPPGPAGAPGDKGEA |
| | | GPSGPAGPTGARGAPGDRGEPGPPG |
| | | PAGFAGPPGADGQPGAKGEPGDAGA |
| | | KGDAGPPGPAGPAGPPGPIGNVGAP |
| | | GPKGARGSAGPPGATGFPGAAGRVG |
| | | PPGPSGNAGPPGPPGPAGKEGSKGP |
| | | RGETGPAGRPGEVGPPGPPGPAGEK |
| | | GAPGADGPAGAPGTPGPQGIAGQRG |
| | | VVGLPGQRGERGFPGLPGPSGEPGK |
| | | QGPSGASGERGPPGPMGPPGLAGPP |
| | | GESGREGAPGAEGSPGRDGSPGAKG |
| | | DRGETGPAGPPGAPGAPGAPGPVGP |
| | | AGKSGDRGETGPAGPAGPIGPVGAR |
| | | GPAGPQGPRGDKGETGEQGDRGIKG |
| | | HRGFSGLQGPPGPPGSPGEQGPSGA |
| | | SGPAGPRGPPGSAGSPGKDGLNGLP |
| | | GPIGPPGPRGRTGDAGPAGPPGPPG |
| | | PPGPPGPPSGGYDLSFLPQPPQEKA |
| | | HDGGRYYRADDANVVRDRDLEVDTT |
| | | LKSLSQQIENIRSPEGSRKNPARTC |
| | | RDLKMCHSDWKSGEYWIDPNQGCNL |
| | | DAIKVFCNMETGETCVYPTQPSVAQ |
| | | KNWYISKNPKEKJRFIVWYGESMTG |
| | | GFQFEYGGQGSDPADVAIQLTFLRL |
| | | MSTEASQNITYHCKNSVAYMDQQTG |
| | | NLKKALLLQGSNEIEIRAEGNSRFT |
| | | YSVTYDGCTSHTGAWGKTVIEYKTT |
| | | KTSRLPIIDVAPLDVGAPDQEFGFD |
| | | VGPACFL |
| SEQ | Soybean | ggttcaacaacacaagcttcaagtt |
| ID | GY1 | ttaaaaggaaaaatgtcagccaaaa |
| NO: 50 | Promoter | actttaaataaaatggtaacaagat |
| | | ctagttccaccttattttatagaga |
| | | gaagaaactaatatataagaactaa |
| | | aaaacagaagaatagaaaaaaaaag |
| | | tattgacaggaaagaaaaagtagct |
| | | gtatgcttataagtactttgaggat |
| | | ttgaattctctcttataaaacacaa |
| | | acacaatttttagattttatttaaa |
| | | taatcatcaatccgattataattat |
| | | ttatatatttttctattttcaaaga |
| | | agtaaatcatgagcttttccaactc |
| | | aacatctattttttttctctcaacc |
| | | tttttcacatcttaagtagtctcac |
| | | cctttatatatataacttatttctt |
| | | accttttacattatgtaacttttat |
| | | caccaaaaccaacaactttaaaatt |
| | | ttattaaatagactccacaagtaac |
| | | ttgacactcttacattcatcgacat |
| | | taacttttatctgttttataaatat |
| | | tattgtgatataatttaatcaaaat |
| | | aaccacaaactttcataaaaggttc |
| | | ttattaagcatggcatttaataagc |
| | | aaaaacaactcaatcactttcatat |
| | | aggaggtagcctaagtacgtactca |
| | | aaatgccaacaaataaaaaaaaagt |
| | | tgctttaataatgccaaaacaaatt |
| | | aataaaacacttacaacaccggatt |
| | | ttttttaattaaaatgtgccattta |
| | | ggataaatagttaatatttttaata |
| | | attatttaaaaagccgtatctacta |
| | | aaatgatttttatttggttgaaaat |
| | | attaatatgtttaaatcaacacaat |
| | | ctatcaagaaattgaaagcgagtct |
| | | aatttttaaattatgaacctgcata |
| | | tataaaaggaaagaaagaatccagg |
| | | aagaaaagaaatgaaaccatgcatg |
| | | gtcccctcgtcatcacgagtttctg |
| | | ccatttgcaatagaaacactgaaac |
| | | acctttctctttgtcacttaattga |
| | | gatgccgaagccacctcacaccatg |
| | | aacttcatgaggtgtagcacccaag |
| | | gcttccatagccatgcatactgaag |
| | | aatgtctcaagctcagcaccctact |
| | | tctgtgacgtgtccctcattcacct |
| | | tcctctcttccctataaataaccac |
| | | gcctcaggttctccgcttc |
| SEQ | Wild Type | MRSLLILVLCFLPLAALGKVFGRCE |
| ID | Chicken | LAAAMKRHGLDNYRGYSLGNWVCAA |
| NO: 51 | Lysozyme | KFESNFNTQATNRNTDGSTDYGILO |
| | | INSRWWCNDGRTPGSRNLCNIPCSA |
| | | LLSSDITASVNCAKKIVSDGNGMNA |
| | | WVAWRNRCKGTDVQAWIRGCRL |
| SEQ | Ovoinhibitor | MTDWVLHHKVGPLDMTTRYIFPLLP |
| ID | | LPFLPHSESKRAVCAPRCSAMRTAR |
| NO: 52 | | QFVQVALALCCFADIAFGIEVNCSL |
| | | YASGIGKDGTSWVACPRNLKPVCGT |
| | | DGSTYSNECGICLYNREHGANVEKE |
| | | YDGECRPKHVTIDCSPYLQVVRDGN |
| | | TMVACPRILKPVCGSDSFTYDNECG |
| | | ICAYNAEHHTNISKLHDGECKLEIG |
| | | SVDCSKYPSTVSKDGRTLVACPRIL |
| | | SPVCGTDGFTYDNECGICAHNAEQR |
| | | THVSKKHDGKCRQEIPEIDCDQYPT |
| | | RKTTGGKLLVRCPRILLPVCGTDGF |
| | | TYDNECGICAHNAQHGTEVKKSHDG |
| | | RCKERSTPLDCTQYLSNTONGEAIT |
| | | ACPFILQEVCGTDGVTYSNDCSLCA |
| | | HNIELGTSVAKKHDGRCREEVPELD |
| | | CSKYKTSTLKDGRQVVACTMIYDPV |
| | | CATNGVTYASECTLCAHNLEQRTNL |
| | | GKRKNGRCEEDITKEHCREFQKVSP |
| | | ICTMEYVPHCGSDGVTYSNRCFFCN |
| | | AYVQSNRTLNLVSMAAC |
| SEQ | Ovoglycoprotein | VVSTAAPTDPRRRMAVSVLLFISVA |
| ID | | LAGVLSTASQACKLEPVKIDLANPO |
| NO: 53 | | LAGKWYFIQVATEVELYQARFANID |
| | | NSYFISTPDVKLNKTNIKEYSQLGD |
| | | LCLSTNTDYVVLENGYEFTDGAKNI |
| | | NNFRIIKSKIDNMLIIDYQYQIEKM |
| | | DYHMLTLFKRNPTASKEEMEIFESY |
| | | TKCLGLDKDKIKAFPRNKSECKEEK |
| | | QINSFNATTQAQDFLEEKVLQNRNI |
| SEQ | Soybean | gtgtgttgtaagtataaautaaaat |
| ID | CGI | aaaaataaaaacaattattatatca |
| NO: 54 | Promoter | aaatggcaaaaacatuaatacgtat |
| | | tatttaagaaaaaaatatgtaataa |
| | | tatatttatattttaatatctatto |
| | | ttatgtattttttaaaaatctatta |
| | | tatattgatcaactaaaatattttt |
| | | atatctacacttattttgcattttt |
| | | atcaattttcttgcgttttttggca |
| | | tatttaataatgactattctttaat |
| | | aatcaatcattattcttacatggta |
| | | catattgttggaaccatatgaagtg |
| | | tccattgcatttgactatgtggata |
| | | gtgttttgatccaggcctccatttg |
| | | ccgcttattaattaatttggtaaca |
| | | gtccgtactaatcagttacttatcc |
| | | ttcctccatcataattaatcttggt |
| | | agtctcgaatgccacaacactgact |
| | | agtctcttggatcataagaaaaagc |
| | | caaggaacaaaagatcacaaaacac |
| | | aatgagagtatcctttgcatagcaa |
| | | tgtctaagttcataaaattcaaaca |
| | | aaaacgcaatcacacacagtggaca |
| | | tcacttatccactagctgatcagga |
| | | tcgccgcgtcaagaaaaaaaaactg |
| | | gaccccaaaagccatgcacaacaac |
| | | acgtactcacaaaggtgtcaatcga |
| | | gcagcccaaaacattcaccaactca |
| | | acccatcatgagcccacacatttgt |
| | | tgtttctaacccaacctcaaactcg |
| | | tattctcttccgccacctcattttt |
| | | gtttatttcaacacccgtcaaactg |
| | | catgccaccccgtggccaaatgtcc |
| | | atgcatgttaacaagacctatgact |
| | | ataaatatctgcaatctcggcccag |
| | | gttttcatc |
| SEQ | Ovomacroglobulin | ALTSGLGPDVYQFLQDMGMKFFTNS |
| ID | | KJRQPTVCTRETVRPPSYFLNAGFT |
| NO: 55 | | ASTHHVKLSAEVAREERGKRHILET |
| | | IREFFPETWIWDIILINSTGKASVS |
| | | YTIPDTITEWKASAFCVLPPNVVEE |
| | | SARASVSVLGDILGSAMONTONLLQ |
| | | MPYGCGEQNMVLFAPNIYVLDYLNE |
| | | TOOLSEDMKSKTIGYLESGYQKQLS |
| | | YKHPDGSY |
| SEQ | Avidin | MVHATSPLLLLLLLSLALVAPGLSA |
| ID | | RKCSLTGKWTNDLGSNMTIGAVNSR |
| NO: 56 | | GEFTGTYITAVTATSNEIKESPLHG |
| | | TONTINKRTQPTFGFTVNWKFSEST |
| | | TVFTGQCFIDRNGKEVLKTMWLLRS |
| | | SVNDIGDDWKATRVGINIFTRLRTO |
| | | KE |
| SEQ | Cystatin | MVGSPRAPLLLLASLIVALALALAV |
| ID | | SPAAAQGPRKGRLLGGLMEADVNEE |
| NO: 57 | | GVQEALSFAVSEFNKRSNDAYQSRV |
| | | VRVVRARKQVVSGMNYFLDVELGRT |
| | | TCTKSQANLDSCPFHNQPHLKREKL |
| | | CSFQVYVVPWMNTINLVKFSCQD |
| SEQ | TMV | ATTTTTACAACAATTACCAACAACA |
| ID | Omega | ACAAACAACAAACAACATTACAATT |
| NO: 58 | | ACATTTACAATT |
| SEQ | AtRbcS2 | TTGCTTCTTCTCTCTTTTTTTGGTT |
| ID | Promoter | CAATATGAACCTTTTGATGTCCACT |
| NO: 59 | | ATCCTTTTATATAATGAAATGATAA |
| | | GGTTTTTGATATGTTATGTGGTTCT |
| | | TGATAACATTATACAATTACTTAAT |
| | | ATCTACATATGAAAGGTTGGAATTT |
| | | TTTTTAAGTCACCACAATAGAGGTG |
| | | ACACGTGTAAGCACCTCGTTAATCT |
| | | TATCTCATCCAAGATGGGGGTAGGA |
| | | AGAGGATATGAATGTATGATTGAGG |
| | | TTGGTTTTGAGTTTTTTTTTTTTTT |
| | | TTTTTTGAGTCCTTCAACTTGTTAT |
| | | TTTAATTTTTTTTTGGTGGGGGAGG |
| | | AGGGGGGTTGAAATATTTATCATAT |
| | | AGTAGTCCAAAGTAAATTGATAGCT |
| | | AGAGTACTTGTTTGCTTGCTTATAT |
| | | TGTCCTCAACTTTATGTAATACCAT |
| | | GATTCCAACTTAGACACTCTTTTAA |
| | | GTTGTAATTTTCATTATTTTCTTTT |
| | | TTTAGAGTTTTATGTTGAATTCGCA |
| | | TAATTTTCAATCGGATAATACAAGA |
| | | AAAATAATATTTTAGTATAATTTTA |
| | | TACATGAAATTTCGGGAAGGTAGGA |
| | | TATACGGATTGTTTGTCGGATCAGA |
| | | GACTTTACTCGTACCTTTGTAACTG |
| | | TTGATCCCAAATACAGATAGTGACT |
| | | CCAGAGTTATATATTATACCTATAG |
| | | AGACTAATATGATTTAGTGTTATTA |
| | | AAATTAGTATTACTAATTAATTGTA |
| | | ATGCCCCATGAATT |
| SEQ | Vector | tctgtgaagacaaactagaattcga |
| ID | bone | gctcggagtgagaccgcagctggca |
| NO: 60 | | cgacaggtttgccgactggaaagcg |
| | | ggcagtgagcgcaacgcaattaatg |
| | | tgagttagctcactcattaggcacc |
| | | ccaggctttacactttatgcttccg |
| | | gctcgtatgttgtgtggaattgtga |
| | | goggataacaatttcacacaggaaa |
| | | cagctatgaccatgattacgccaag |
| | | cttgcatgcctgcaggtcgactcta |
| | | gaggatccccgggtaccgagctcga |
| | | attcactggccgtcgttttacaacg |
| | | tcgtgactgggaaaaccctggcgtt |
| | | acccaacttaatcgccttgcagcac |
| | | atccccctttcgccagctggcgtaa |
| | | tagcgaagaggcccgcaccgatcgc |
| | | ccttcccaacagttgcgcagcctga |
| | | atggcgaatggcgcctgatgcggta |
| | | ttttctccttacgcatctgtgcggt |
| | | atttcacaccgcatatggtgcacto |
| | | tcagtacaatctgctctgatgccgc |
| | | atagttaagccagccccgacacccg |
| | | ccaacacccgctgacgcgccctgac |
| | | gggcttgtctgctcccggcatccgc |
| | | ttacagacaagctgtgacggtctca |
| | | cgctttacttgtcttctgcacgaag |
| | | tggtttaaactatcagtgtttgaca |
| | | ggatatattggcgggtaaacctaag |
| | | agaaaagagcgtttattagaataat |
| | | cggatatttaaaagggcgtgaaaag |
| | | gtttatccgttcgtccatttgtatg |
| | | tgcatgccaaccacagggttcccca |
| | | gatcaggcgctggctgctgaacccc |
| | | cagccggaactgaccccacaaggcc |
| | | ctagcgtttgcaatgcaccaggtca |
| | | tcattgacccaggcgtgttccacca |
| | | ggccgctgcctcgcaactcttcgca |
| | | ggcttcgccgacctgctcgcgccac |
| | | ttcttcacgcgggtggaatccgatc |
| | | cgcacatgaggcggaaggtttccag |
| | | cttgagcgggtacggctcccggtgc |
| | | gagctgaaatagtcgaacatccgtc |
| | | gggccgtcggcgacagcttgcggta |
| | | cttctcccatatgaatttcgtgtag |
| | | tggtcgccagcaaacagcacgacga |
| | | tttcctcgtcgatcaggacctggca |
| | | acgggacgttttcttgccacggtcc |
| | | aggacgcggaagcggtgcagcagcg |
| | | acaccgattccaggtgcccaacgcg |
| | | gtcggacgtgaagcccatcgccgtc |
| | | gcctgtaggcgcgacaggcattcct |
| | | cggccttcgtgtaataccggccatt |
| | | gatcgaccagcccaggtcctggcaa |
| | | agctcgtagaacgtgaaggtgatcg |
| | | gctcgccgataggggtgcgcttcgc |
| | | gtactccaacacctgctgccacacc |
| | | agttcgtcatcgtcggcccgcagct |
| | | cgacgccggtgtaggtgatcttcac |
| | | gtccttgttgacgtggaaaatgacc |
| | | ttgttttgcagcgcctcgcgcggga |
| | | ttttcttgttgcgcgtggtgaacag |
| | | ggcagagcgggccgtgtcgtttggc |
| | | atcgctcgcatcgtgtccggccacg |
| | | gcgcaatatcgaacaaggaaagctg |
| | | catttccttgatctgctgcttcgtg |
| | | tgtttcagcaacgcggcctgcttgg |
| | | cctcgctgacctgttttgccaggtc |
| | | ctcgccggcggtttttcgcttcttg |
| | | gtcgtcatagttcctcgcgtgtcga |
| | | tggtcatcgacttcgccaaacctgc |
| | | cgcctcctgttcaagacgacgcgaa |
| | | cgctccacggcggccgatggcgcgg |
| | | gcagggcagggggagccagttgcac |
| | | gctgtcgcgctcgatcttggccgta |
| | | gottgctggaccatcgagccgacgg |
| | | actggaaggtttcgcggggcgcacg |
| | | catgacggtgcggcttgcgatggtt |
| | | tcggcatcctcggcggaaaaccccg |
| | | cgtcgatcagttcttgcctgtatge |
| | | cttccggtcaaacgtccgattcatt |
| | | caccctccttgcgggattgccccga |
| | | ctcacgccggggcaatgtgccctta |
| | | ttcctgatttgacccgcctggtgcc |
| | | ttggtgtccagataatccaccttat |
| | | cggcaatgaagtcggtcccgtagac |
| | | cgtctggccgtccttctcgtacttg |
| | | gtattccgaatcttgccctgcacga |
| | | ataccagcgaccccttgcccaaata |
| | | cttgccgtgggcctcggcctgagag |
| | | ccaaaacacttgatgcggaagaagt |
| | | cggtgcgctcctgcttgtcgccggc |
| | | atcgttgcgccacatctaggatctg |
| | | ccaggaaccgtaaaaaggccgcgtt |
| | | gctggcgtttttccataggctccgc |
| | | ccccctgacgagcatcacaaaaatc |
| | | gacgctcaagtcagaggtggcgaaa |
| | | cccgacaggactataaagataccag |
| | | gcgtttccccctggaagctccctcg |
| | | tgcgctctcctgttccgaccctgcc |
| | | gettaccggatacctgtccgccttt |
| | | ctcccttcgggaagcgtggcgcttt |
| | | ctcatagctcacgctgtaggtatct |
| | | cagttcggtgtaggtcgttcgctcc |
| | | aagctgggctgtgtgcacgaacccc |
| | | ccgttcagcccgaccgctgcgcctt |
| | | atccggtaactatcgtcttgagtcc |
| | | aacccggtaagacacgacttatcgc |
| | | cactggcagcagccactggtaacag |
| | | gattagcagagcgaggtatgtagge |
| | | ggtgctacagagttcttgaagtggt |
| | | ggcctaactacggctacactagaag |
| | | gacagtatttggtatctgcgctctg |
| | | ctgaagccagttaccttcggaaaaa |
| | | gagttggtagctcttgatccggcaa |
| | | acaaaccaccgctggtagcggtggt |
| | | ttttttgtttgcaagcagcagatta |
| | | cgcgcagaaaaaaaggatctcaaga |
| | | agatcctttgatcttttctacgggg |
| | | tctgacgctcagtggaacgaaaact |
| | | cacgttaagggattttggtcatgag |
| | | attatcaaaaaggatcttcacctag |
| | | atccttttaaattaaaaatgaagtt |
| | | ttaaatcaatctaaagtatatatga |
| | | gtaaacttggtctgacagttaccaa |
| | | tgcttaatcagtgaggcacctatct |
| | | cagcgatctgtctatttcgttcatc |
| | | catagttgcctgactccccgtcgtg |
| | | tagataactacgatacgggagggct |
| | | taccatctggccccagtgctgcaat |
| | | gataccgcgagaaccacgctcaccg |
| | | gctccagatttatcagcaataaacc |
| | | agccagccggaagggccgagcgcag |
| | | aagtggtcctgcaactttatccgcc |
| | | tccatccagtctattaattgttgcc |
| | | gggaagctagagtaagtagttcgcc |
| | | agttaatagtttgcgcaacgttgtt |
| | | gccattgctacaggcatcgtggtgt |
| | | cacgctcgtcgtttggtatggcttc |
| | | attcagctccggttcccaacgatca |
| | | aggcgagttacatgatcccccatgt |
| | | tgtgcaaaaaagcggttagctcctt |
| | | cggtcctccgatcgttgtcagaagt |
| | | aagttggccgcagtgttatcactca |
| | | tggttatggcagcactgcataattc |
| | | tcttactgtcatgccatccgtaaga |
| | | tgcttttctgtgactggtgagtact |
| | | caaccaagtcattctgagaatagtg |
| | | tatgcggcgaccgagttgctcttgc |
| | | ccggcgtcaatacgggataataccg |
| | | cgccacatagcagaactttaaaagt |
| | | gctcatcattggaaaacgttcttcg |
| | | gggcgaaaactctcaaggatcttac |
| | | cgctgttgagatccagttcgatgta |
| | | acccactcgtgcacccaactgatct |
| | | tcagcatcttttactttcaccagcg |
| | | tttctgggtgagcaaaaacaggaag |
| | | gcaaaatgccgcaaaaaagggaata |
| | | agggcgacacggaaatgttgaatac |
| | | tcatactcttcctttttcaatatta |
| | | ttgaagcatttatcagggttattgt |
| | | ctcatgagoggatacatatttgaat |
| | | gtatttagaaaaataaacaaatagg |
| | | ggttccgcgcacgaattggccagcg |
| | | ctgccatttttggggtgaggccgtt |
| | | cgcggccgaggggcgcagcccctgg |
| | | ggggatgggaggcccgcgttagcgg |
| | | gccgggagggttegagaaggggggg |
| | | caccccccttcggcgtgcgcggtca |
| | | cgcgcacagggcgcagccctggtta |
| | | aaaacaaggtttataaatattggtt |
| | | taaaagcaggttaaaagacaggtta |
| | | gcggtggccgaaaaacgggcggaaa |
| | | cccttgcaaatgctggattttctgc |
| | | ctgtggacagcccctcaaatgtcaa |
| | | taggtgcgcccctcatctgtc |
| | | agcactctgcccctcaagtgtcaag |
| | | gatcgcgcccctcatctgtcagtag |
| | | tcgcgcccctcaagtgtcaataccg |
| | | cagggcacttatccccaggcttgtc |
| | | cacatcatctgtgggaaactcgcgt |
| | | aaaatcaggcgttttcgccgatttg |
| | | cgaggctggccagctccacgtcgcc |
| | | ggccgaaatcgagcctgcccctcat |
| | | ctgtcaacgccgcgccgggtgagtc |
| | | ggcccctcaagtgtcaacgtccgcc |
| | | cctcatctgtcagtgagggccaagt |
| | | tttccgcgaggtatccacaacgccg |
| | | gcggccgcggtgtctcgcacacggc |
| | | ttcgacggcgtttctggcgcgtttg |
| | | cagggccatagacggccgccagccc |
| | | agcggcgagggcaaccagcccggtg |
| | | agcgtcgcaaaggagatcctgatct |
| | | gactgatgggctgcctgtatcgagt |
| | | ggtgattttgtgccgagctgccggt |
| | | cggggagctgttggctggctggtgg |
| | | caggatatattgtggtgtaaacaaa |
| | | ttgacgcttagacaacttaataaca |
| | | cattgcggacgtttttaatgtactg |
| | | gggtggatgcagtgggccccac |
| SEQ | pUC Origin | gccaggaaccgtaaaaaggccgcgt |
| ID | | tgctggcgtttttccataggctccg |
| NO: 61 | | cccccctgacgagcatcacaaaaat |
| | | cgacgctcaagtcagaggtggcgaa |
| | | acccgacaggactataaagatacca |
| | | ggcgtttccccctggaagctccctc |
| | | gtgcgctctcctgttccgaccctgc |
| | | cgcttaccggatacctgtccgcctt |
| | | tctcccttcgggaagcgtggcgctt |
| | | tctcatagctcacgctgtaggtatc |
| | | tcagtteggtgtaggtcgttcgctc |
| | | caagctgggctgtgtgcacgaaccc |
| | | cccgttcagcccgaccgctgcgcct |
| | | tatccggtaactatcgtcttgagtc |
| | | caacccggtaagacacgacttatcg |
| | | ccactggcagcagccactggtaaca |
| | | ggattagcagagcgaggtatgtagg |
| | | cggtgctacagagttcttgaagtgg |
| | | tggcctaactacggctacactagaa |
| | | ggacagtatttogtatctgcgctct |
| | | gctgaagccagttaccttcggaaaa |
| | | agagttggtagctcttgatccggca |
| | | aacaaaccaccgctggtagcggtgg |
| | | tttttttgtttgcaagcagcagatt |
| | | acgcgcagaaaaaaaggatctcaag |
| | | aagatcctttgatcttttctacggg |
| | | gtctgacgctcagtggaacgaaaac |
| | | tcacgttaagggattttggtcatga |
| | | gattatcaaaaaggatcttcaccta |
| | | gatccttttaaattaaaaatgaagt |
| | | tttaaatcaatctaaagtatatatg |
| | | agtaaacttggtctg |
| SEQ | oriV | ccgggagggttcgagaagggggggc |
| ID | | accccccttcggcgtgcgcggtcac |
| NO: 62 | | gcgcacagggcgcagccctggttaa |
| | | aaacaaggtttataaatattggttt |
| | | aaaagcaggttaaaagacaggttag |
| | | cggtggccgaaaaacgggcggaaac |
| | | ccttgcaaatgctggattttctgcc |
| | | tgtggacagcccctcaaatgtcaat |
| | | aggtgcgcccctcatctgtcagcac |
| | | tctgcccctcaagtgtcaaggatog |
| | | cgcccctcatctgtcagtagtcgcg |
| | | cccctcaagtgtcaataccgcaggg |
| | | cacttatccccaggcttgtccacat |
| | | catctgtgggaaactcgcgtaaaat |
| | | caggcgttttcgccgatttgcgagg |
| | | ctggccagctccacgtcgccggccg |
| | | aaatcgagcctgcccctcatotgtc |
| | | aacgccgcgccgggtgagtcggccc |
| | | ctcaagtgtcaacgtccgcccctca |
| | | tctgtcagtgagggccaagttttcc |
| | | gcgaggtatccacaacgccggcggc |
| | | cgcggtgtctcgcacacggcttcga |
| | | cggcgtttctggcgcgtttgcaggg |
| | | ccatagacggccgccagcccagcgg |
| | | cgagggcaaccagcccgg |
| SEQ | lacZ | ccgactggaaagcgggcagtgagcg |
| ID | | caacgcaattaatgtgagttagctc |
| NO: 63 | | actcattaggcaccccaggctttac |
| | | actttatgcttccggctcgtatgtt |
| | | gtgtggaattgtgagcggataacaa |
| | | tttcacacaggaaacagctatgacc |
| | | atgattacgccaagcttgcatgcct |
| | | gcaggtcgactctagaggatccccg |
| | | ggtaccgagctcgaattcactggcc |
| | | gtcgttttacaacgtcgtgactggg |
| | | aaaaccctggcgttacccaacttaa |
| | | tcgccttgcagcacatccccctttc |
| | | gccagctggcgtaatagcgaagagg |
| | | cccgcaccgatcgcccttcccaaca |
| | | gttgcgcagcctgaatggcgaatgg |
| | | cgcctgatgcggtattttctcctta |
| | | cgcatctgtgcggtatttcacaccg |
| | | catatggtgcactctcagtacaatc |
| | | tgctctgatgccgcatagttaagcc |
| | | agccccgacacccgccaa?acccgc |
| | | tgacgcgccctgacgggcttgtctg |
| | | ctcccggcatcc |
| SEQ | RB | aaactatcagtgtttgacaggatat |
| ID | | attggcgggtaaacctaagagaaaa |
| NO: 64 | | gagcgtttattagaataatcggata |
| | | tttaaaagggcgtgaaaaggtttat |
| | | ccgttcgtccatttgtatgtgcatg |
| | | cca |
| SEQ | HDEL | CACGACGAATTGTAA |
| ID | | |
| NO: 65 | | |
| SEQ | HsFam20C | CTAGATTTACTCCCCCGACTGGAGC |
| ID | | GACGTGGAGCGCGTCCGTCTGGCGA |
| NO: 66 | | ACCAGGATGTTCTTGTGCGCAACCA |
| | | GCTGCGGAAGTCGCTGCGCCAGGTT |
| | | GGGCACAAGTTCGAGGGAGGCCTGG |
| | | GGAGCCACCAGCTGCTAGCTCCGCT |
| | | GCGGGCGATGCCGGATGGCCAAATA |
| | | AACACACTCTAAGAATACTACAGGA |
| | | CTTCTCAAGCGACCCTTCCTCTAAT |
| | | CTCTCATCACACTCACTTGAGAAGT |
| | | TACCTCCCGCAGCCGAGCCGGCCGA |
| | | GCGAGCGCTTAGGGGGCGTGACCCC |
| | | GGGGCATTACGACCTCACGATCCTG |
| | | CACACAGGCCTTTGTTACGTGATCC |
| | | AGGGCCGCGAAGAAGCGAATCCCCT |
| | | CCTGGACCCGGAGGCGACGCATCCC |
| | | TTCTGGCGAGGTTATTTGAACACCC |
| | | ATTGTACAGAGTCGCCGTGCCTCCC |
| | | CTGACTGAAGAGGACGTCTTGTTCA |
| | | ACGTTAACTCTGACACCAGACTTAG |
| | | TCCTAAAGCGGCCGAGAATCCTGAT |
| | | TGGCCGCATGCCGGGGCAGAGGGCG |
| | | CTGAGTTCCTTAGCCCAGGAGAGGC |
| | | CGCGGTGGACTCCTATCCGAACTGG |
| | | TTGAAATTCCATATTGGAATAAATC |
| | | GATACGAACTATATTCTCGACACAA |
| | | TCCGGCCATCGAGGCCCTATTACAT |
| | | GATCTGTCCTCACAACGTATTACGT |
| | | CAGTGGCCATGAAGAGCGGCGGCAC |
| | | GCAACTAAAGCTGATTATGACATTT |
| | | CAAAACTATGGTCAAGCCCTGTTTA |
| | | AGCCAATGAAGCAGACAAGAGAACA |
| | | GGAGACGCCTCCCGACTTCTTCTAC |
| | | TTTAGTGATTATGAACGACATAATG |
| | | CTGAAATAGCGGCCTTTCACCTAGA |
| | | CAGGATCTTAGACTTCCGTAGGGTT |
| | | CCCCCAGTTGCAGGTAGGATGGTTA |
| | | ACATGACTAAAGAGATAAGGGATGT |
| | | AACCAGGGATAAGAAGCTATGGAGG |
| | | ACGTTCTTTATTTCTCCTGCGAACA |
| | | ATATCTGTTTTTACGGCGAGTGCTC |
| | | CTACTATTGCAGCACAGAACATGCA |
| | | CTATGTGGAAAGCCTGACCAGATTG |
| | | AAGGTTCCCTGGCGGCCTTTCTTCC |
| | | AGACCTCTCTCTTGCGAAACGTAAA |
| | | ACTTGGCGTAATCCATGGAGACGAA |
| | | GCTATCACAAACGTAAGAAAGCGGA |
| | | GTGGGAGGTAGACCCCGATTACTGC |
| | | GAGGAGGTTAAGCAAACCCCGCCGT |
| | | ATGACTCTAGCCACCGAATATTAGA |
| | | CGTTATGGATATGACGATCTTTGAT |
| | | TTTCTTATGGGCAACATGGATCGTC |
| | | ACCACTACGAAACGTTCGAGAAGTT |
| | | TGGAAATGAGACGTTTATTATCCAT |
| | | CTGGACAACGGCAGAGGCTTTGGGA |
| | | AATATAGTCATGACGAATTATCCAT |
| | | TCTTGTTCCTTTACAGCAATGCTGC |
| | | AGAATCAGGAAATCAACGTATTTAC |
| | | GTCTCCAACTCCTGGCAAAAGAGGA |
| | | ATATAAGCTTAGCCTCCTAATGGCT |
| | | GAGTCTCTGAGGGGCGACCAAGTTG |
| | | CGCCTGTTCTGTATCAACCACATTT |
| | | AGAAGCACTTGATAGACGTTTGCGT |
| | | GTTGTACTTAAAGCTGTCCGAGACT |
| | | GTGTCGAGAGAAATGGCCTTCATTC |
| | | AGTTGTGGACGACGATCTAGATACC |
| | | GAACATAGGGCAGCGTCCGCACGT |
| SEQ | HsFam20A | CAACTCAGGCCAAGGGAACGTCCCC |
| ID | | GTGGGTGCCCGTGTACCGGAAGGGC |
| NO: 67 | | ATCTAGTTTGGCGCGAGATTCTGCT |
| | | GCTGCGGCGTCAGACCCAGGGACAA |
| | | TTGTGCATAATTTTTCCCGTACTGA |
| | | GCCAAGAACAGAGCCGGCCGGTGGG |
| | | AGCCATAGCGGAAGTTCATCTAAGT |
| | | TGCAAGCACTATTCGCGCATCCTTT |
| | | GTATAATGTACCTGAAGAACCTCCT |
| | | CTTTTAGGAGCGGAGGACTCACTCT |
| | | TAGCCAGCCAGGAAGCCCTTAGATA |
| | | CTACCGTAGAAAGGTTGCCCGATGG |
| | | AACCGTAGGCATAAAATGTACCGTG |
| | | AGCAAATGAATTTAACTAGTCTTGA |
| | | TCCTCCTCTCCAACTGAGGCTTGAG |
| | | GCTAGTTGGGTTCAGTTCCATTTGG |
| | | GTATCAATCGACACGGCCTTTACTC |
| | | TAGGTCAAGTCCTGTGGTATCTAAA |
| | | CTTCTTCAAGACATGCGTCATTTTC |
| | | CGACAATATCTGCCGATTACTCCCA |
| | | GGATGAGAAGGCTTTGCTTGGAGCA |
| | | TGTGACTGTACCCAGATAGTAAAAC |
| | | CTAGTGGCGTCCACTTAAAACTGGT |
| | | CCTCCGTTTTAGCGATTTTGGGAAA |
| | | GCTATGTTTAAACCCATGCGACAAC |
| | | AACGTGATGAAGAAACGCCAGTTGA |
| | | TTTCTTCTACTTCATTGACTTCCAG |
| | | CGTCACAACGCGGAGATAGCAGCAT |
| | | TCCACTTAGACAGAATTCTAGACTT |
| | | TCGTCGAGTCCCCCCTACCGTTGGC |
| | | CGTATAGTGAATGTAACTAAAGAGA |
| | | TTTTGGAGGTGACAAAAAACGAGAT |
| | | CTTACAAAGCGTCTTTTTTGTATCC |
| | | CCGGCCTCCAACGTCTGTTTTTTTG |
| | | CGAAATGTCCCTATATGTGCAAAAC |
| | | TGAATATGCGGTCTGCGGCAACCCC |
| | | CATTTACTCGAAGGTAGTCTCAGTG |
| | | CATTTCTCCCCAGTCTCAACTTAGC |
| | | TCCACGTCTAAGCGTGCCAAACCCT |
| | | TGGATTAGGAGCTATACGCTAGCGG |
| | | GCAAGGAGGAGTGGGAGGTGAACCC |
| | | ACTTTATTGTGACACAGTCAAGCAA |
| | | ATCTATCCTTATAATAACTCACAGA |
| | | GACTACTCAATGTCATTGATATGGC |
| | | TATCTTCGATTTCCTGATAGGAAAC |
| | | ATGGATAGGCACCATTATGAGATGT |
| | | TCACCAAGTTCGGGGACGATGGTTT |
| | | TCTGATACATCTAGACAACGCGCGT |
| | | GGCTTCGGGCGACACTCCCACGACG |
| | | AAATCTCCATTCTTAGCCCCCTGAG |
| | | CCAGTGTTGCATGATCAAAAAGAAA |
| | | ACACTTCTGCATCTTCAGCTCCTCG |
| | | CTCAAGCTGACTATCGACTTTCCGA |
| | | CGTGATGCGAGAATCACTGCTTGAA |
| | | GATCAGCTCAGCCCAGTGCTTACTG |
| | | AACCGCACCTACTGGCACTAGATCG |
| | | TAGATTACAGACGATCTTGAGGACA |
| | | GTCGAGGGGTGCATCGTTGCTCACG |
| | | GCCAACAAAGCGTCATAGTTGATGG |
| | | GCCTGTGGAACAGCTAGCTCCGGAT |
| | | TCCGGACAAGCAAATTTAACCAGC |
| SEQ | BtFam20C | CTCCTACCCAAATTAGAACGATCTG |
| ID | | CTGCACGTCCGAGCGGCGAACCTGG |
| NO: 68 | | CTGTAGCTGCGCACAGCCAGCTGCC |
| | | GAAGCTGCCGCGCCTGGATGGGCTC |
| | | AAGCTAGGGGTCATCCCGGTGGAGA |
| | | ACTTGAAGCGGCCGCTAGCGCCGCC |
| | | GGGGATGCAGGCTGGCCAAATAAGC |
| | | ACACTCTGAGGATTCTGCAAGACTT |
| | | CAGTTCCGACCCCAGTTCCAACCTA |
| | | ACGAGCCACTCACTGGAAAAGCTGC |
| | | CTCCGGCTGCCGAAGCTGCGGAAGG |
| | | TGCACCGCCAGGCCAAGATCCAGGA |
| | | GTTCGTAGACCTCCCGACCCAGCGC |
| | | ATAG |
| | | GCCACTCCCGCGAGATCCGGGTCCT |
| | | AGAGGCCCTGTCTTGCCCCCAGGTC |
| | | TTAGCGGAGACGGGTCCTTACTTAC |
| | | GCGTCTTTTCCAACACCCGCTATAC |
| | | CAGGTGCCCATACCGCCCCTAACAG |
| | | AAGGCGATGTTCTCTTTAATGTCAA |
| | | TAGCGATATAAGATTCAACCCCAAA |
| | | GCTGCAACCGCCGAGAACCCAGATT |
| | | GGCCACACGAGGGGCCGGAAGATGA |
| | | GTTTTTACCTACTGGTGAAGCGGCA |
| | | GTTGACTCTTACCCGAATTGGCTGA |
| | | AGTTTCATATTGGGATCAATAGATA |
| | | CGAGCTTTACAGCCGACATAATCCG |
| | | GCCGTGGGAGCGCTCTTACAAGACC |
| | | TCGGGACGCAAAAGATTACTTCTGT |
| | | CGCTATGAAATCTGGCGGGACACAG |
| | | CTCAAACTTATTATGACTTTCCAGA |
| | | ATTATGGCCAAGCTCTGTTCAAGCC |
| | | GATGAAGCAGACTAGAGAGCAGGAG |
| | | ACACCCCCTGACTTCTTCTACTTCA |
| | | GCGACTATGAAAGGCATAATGCAGA |
| | | AATTGCGGCATTCCACCTTGATAGG |
| | | ATCTTAGACTTCCGAAGGGTACCAC |
| | | CGGTAGCAGGTAGACTAGTCAATAT |
| | | GACTAAAGAGATTAGAGATGTCACT |
| | | CGTGACAAGAAACTATGGCGTACAT |
| | | TCTTTATAAGCCCTGCTAACAATGT |
| | | ATGCTTTTATGGCGAATGTTCTTAC |
| | | TATTGCTCTACAGAACATGCACTGT |
| | | GTGGAAAACCCGACCAGATTGAGGG |
| | | GTCACTAGCCGCATTTCTGCCAGAC |
| | | TTGGCATTGGCCAAGCGTAAGACGT |
| | | GGCGTAATCCGTGGCGACGTAGTTA |
| | | CCACAAGAGAAAGAAGGCGGAGTGG |
| | | GAAGTAGACCCAGACTACTGCGAGG |
| | | AGGTTAGACAAACACCTCCATATGA |
| | | TTCTAGTCATAGACTGTTGGATGTT |
| | | ATGGACATGACAATTTTTGATTTTC |
| | | TCATGGGGAACATGGATCGTCACCA |
| | | CTACGAAACCTTTGAGAAATTCGGC |
| | | AATGAGACATTCATTATCCACTTAG |
| | | ATAATGGTCGAGGTTTTGGCAAACA |
| | | CAGCCATGACGAACTATCTATATTA |
| | | GTGCCTTTACAACAGTGCTGTAGAA |
| | | TCCGAAGGTCTACCTATTTGAGACT |
| | | TCAACTGTTGGCCCAAGAGGAGCAT |
| | | CGTCTATCACTTTTAATGGCCGAGG |
| | | CTCTAAGGGCTGATCGTGTGGCTCC |
| | | CGTACTCTTTCAGCCTCACTTAGAG |
| | | GCTTTAGATCGTCGACTTCGTATAG |
| | | TGCTTCGAGCGGTAGGCGATTGCGT |
| | | GGAGAAAGATGGACTGCACAGTGTT |
| | | GTAGAGGATGATTTGGGGCCTGAGC |
| | | ACAGGGCGGCCGCGGGACGT |
| SEQ | BtFam20A | CACCTTGGTCCCAGGGTACGATCCA |
| ID | | GACTGCAACCGAGGGAACGACCGTT |
| NO: 69 | | GGGGTGCCCTTGTGCGCGTCGTGCC |
| | | GCTAGTCCTGCTCCGGGACCTGCCC |
| | | CGAGTGCGCCCAGGCGTGTGGAACC |
| | | AAGCGGCGGTGGCGATCCAGGGTCC |
| | | AAACTCAGGGCACTTTTCGCACACC |
| | | CATTGTATAACGAACCAGAGGAACC |
| | | TCCGCTGCTTGGGCCCGAGGATAGC |
| | | CTGCTGGCGGGTCCCGAAGCATTAC |
| | | GTTATTACCGAAGAAAAGTAGCAAG |
| | | GTGGAATAGACGTCGAAAGATGTAC |
| | | AAAGAACAATTAAACCTAACCAGCC |
| | | CAGAACCGCCGGTGCAATTGAGACA |
| | | AGAGGCATCATGGGTACAATTTCAC |
| | | TTGGGGATCAATAGGCATGGGCTGT |
| | | ATCCTCGTAGCTCTCCGGTGGTTTC |
| | | TAAACTCCTTCAGGATATGAGACAC |
| | | TTCCCTACGATCAGCGCGGATTATA |
| | | GCCAGGACGAAAAGGCTCTTCTTGG |
| | | CGCATGTGACTGTTCTCAAATTGTT |
| | | AAGCCCTCCGGCGTTCATCTGAAGC |
| | | TCGTACTTCGTTTTTCTGATTTTGG |
| | | GAAGGCGATGTTTAAACCCATGAGA |
| | | CAGCAGAGGGACGAGGAGACGCCGG |
| | | AGGACTTCTTCTATTTCATCGACTT |
| | | CCAGAGACATAATGCCGAAATTGCT |
| | | GCCTTCCATCTAGATCGTATTTTGG |
| | | ACTTTAGACGTGTCCCGCCAACAGT |
| | | AGGGAGGCTCGTAAATGTGACAAGG |
| | | GAAATACTAGAAGTCACTCGTAATG |
| | | AGATTTTACAAAGCGTGTTTTTCGT |
| | | GAGTCCGGCAAACAATGTGTGCTTC |
| | | TTTGCGAAATGCCCTTATATGTGCA |
| | | AGACGGAGTATGCTGTTTGCGGGTC |
| | | ACCCCACCTACTAGAAGGTTCTTTA |
| | | AGTGCCTTCCTTCCCAGCCTCAACC |
| | | TCGCTCCACGTTTCAGTATGCCTAG |
| | | TCCATGGATCAGGTCATATTCCCTA |
| | | GCTGGTCGTGAAGAGTGGGAGGTAA |
| | | ACCCGCTATACTGCGAAGCGGTGAA |
| | | ACAGGCTTACCCCCACAATAGCAGT |
| | | TCACGTCTATTAAATATTATCGATA |
| | | TGGCGATCTTCGACTTTCTAATAGG |
| | | GAACATGGACAGGCACCACTACGAA |
| | | ATGTTCACTAAGTTCGGTGAGGACG |
| | | GGTTCCTAATACATTTGGATAATGC |
| | | CCGTGGTTTTGGGAGACATTCACAC |
| | | GATGAAGTCTCTATTTTAGCCCCTT |
| | | TATTCCAATGTTGTAGGATAAAAAG |
| | | AAAAACCCTGCTGCATCTTCAACTC |
| | | CTGGCTCAGGCTGATTACCGTCTTT |
| | | CAGACGTTATGCGTGAGTCTTTGCT |
| | | AGAGGACCAATTGAGTCCTGTACTA |
| | | ACAGAACCACATCTGCTAGCTTTGG |
| | | ACAGACGTTTGCAGACTGTGCTAAG |
| | | AACGGTCCAGGATTGCATCGAGGCC |
| | | CATGGGGAACAATCAGTGGTAGCCG |
| | | ACGGCCCGGTGGGCCAATGGGCTCC |
| | | CGACAGTAGTAGAGCGAACGCAACT |
| | | TCT |
| SEQ | At UMAS | ATTTTTCAAATCAGTGCGCAAGACG |
| ID | | TGACGTAAGTATCCGAGTCAGTTTT |
| NO: 70 | | TATTTTTCTACTAATTTGGTCGTTT |
| | | ATTTCGGCGTGTAGGA |
| | | CATGGCAACCGGGCCTGAATTTCGC |
| | | GGGTATTCTGTTTCTATTCCAACTT |
| | | TTTCTTGATCCGCAGCCATTAACGA |
| | | CTTTTGAATAGATACGCTGACACGC |
| | | CAAGCCTCGCTAGTCAAAAGTGTAC |
| | | CAAACAACGCTTTACAGCAAGAACG |
| | | GAATGCGCGTGACGCTCGCGGTGAC |
| | | GCCATTTCGCCTTTTCAGAAATGGA |
| | | TAAATAGCCTTGCTTCCTATTATAT |
| | | CTTCCCAAATTACCAATACATTACA |
| | | CTAGCATCTGAATTTCATAACCAAT |
| | | CTCGATACACCAAATCG |
| SEQ | ARA12 | ATGAGTTCTAGCTTTTTATCATCCA |
| ID | Signal | CTGCGTTCTTCCTGCTCCTTTGCTT |
| NO: 71 | Peptide | AGGCTTCTGCCATGTGTCATCT |
| SEQ | A1282 | ATGGCAAACAAGCTCTTCCTCGTCT |
| ID | Signal | GCGCAACTTTCGCCCTCTGCTTCCT |
| NO: 72 | Peptide | CCTCACCAACGCT |
| SEQ | HA Tag, | TCGTACCCCTACGACGTTCCTGACT |
| ID | 6His Tag, | ACGCCCATCATCACCATCACCACCA |
| NO: 73 | HDEL | TGATGAGTTGTAG |
| SEQ | Octopine | GTCCTGCTTTAATGAGATATGCGAG |
| ID | (OCS) | AAGCCTATGATCGCATGATATTTGC |
| NO: 74 | Terminator | TTTCAATTCTGTTGTGCACGTTGTA |
| | | AAAAACCTGAGCATGTGTAGCTCAG |
| | | ATCCTTACCGCCGGTTTCGGTTCAT |
| | | TCTAATGAATATATCACCCGTTACT |
| | | ATCGTATTTTTATGAATAATATTCT |
| | | CCGTTCAATTTACTGATTGTACCCT |
| | | ACTACTTATATGTACAATATTAAAA |
| | | TGAAAACAATATATTGTGCTGAATA |
| | | GGTTTATAGCGACATCTATGATAGA |
| | | GCGCCACAATAACAAACAATTGCGT |
| | | TTTATTATTACAAATCCAATTTTAA |
| | | AAAAAGCGGCAGAACCGGTCAAACC |
| | | TAAAAGACTGATTACATAAATCTTA |
| | | TTCAAATTTCAAAAGTGCCCCAGGG |
| | | GCTAGTATCTACGACACACCGAGCG |
| | | GCGAACTAATAACGCTCACTGAAGG |
| | | GAACTCCGGTTCCCCGCCGGCGCGC |
| | | ATGGGTGAGATTCCTTGAAGTTGAG |
| | | TATTGGCCGTCCGCTCTACCGAAAG |
| | | TTACGGGCACCATTCAACCCGGTCC |
| | | AGCACGGCGGCCGGGTAACCGACTT |
| | | GCTGCCCCGTGCAGGTCAAACCTTG |
| | | ACAGTGACGACAAATCGTTGGGCGG |
| | | GTCCAGGGCGAATTTTGCGACAACA |
| | | TGTCGAGGCTCAGCAGGAC |
| SEQ | mScarlet | ATGGGTGGTGGAGGAAGTGGAGGTG |
| ID | Coding | GAGGAAGTGTCAGTAAAGGAGAAGC |
| NO: 75 | Sequence | TGTCATAAAGGAGTTCATGAGGTTT |
| | | AAGGTACACATGGAAGGGTCAATGA |
| | | ACGGGCATGAATTTGAGATCGAGGG |
| | | GGAGGGAGAAGGAAGGCCTTATGAG |
| | | GGCACTCAAACTGCTAAGTTGAAGG |
| | | TCACTAAGGGGGGGCCTCTTCCTTT |
| | | TTCTTGGGACATACTCAGCCCTCAG |
| | | TT |
| | | CATGTATGGCTCAAGGGCCTTTACA |
| | | AAACACCCTGCCGATATCCCCGATT |
| | | ACTATAAGCAGAGCTTTCCCGAAGG |
| | | CTTCAAGTGGGAGAGGGTTATGAAT |
| | | TTTGAAGATGGCGGTGCTGTTACAG |
| | | TCACACAGGACACAAGTCTTGAGGA |
| | | TGGGACCCTGATTTATAAAGTTAAG |
| | | CTCAGGGGGACAAATTTCCCACCAG |
| | | ATGGACCAGTCATGCAGAAGAAAAC |
| | | TATGGGATGGGAAGCCTCTACCGAG |
| | | CGTCTTTATCCCGAAGATGGCGTTC |
| | | TTAAAGGCGATATTAAGATGGCTTT |
| | | GAGGCTCAAAGATGGCGGTCGTTAC |
| | | CTGGCCGATTTCAAAACCACTTACA |
| | | AAGCCAAAAAACCAGTACAGATGCC |
| | | AGGCGCTTACAACGTTGATCGTAAG |
| | | CTGGACATCACTTCCCACAACGAGG |
| | | ATTACACAGTTGTTGAACAGTATGA |
| | | GAGATCAGAGGGTAGACATAGTACC |
| | | GGCGGAATGGATGAGCTGTACAAAA |
| | | GTTCG |
| SEQ | NOS | GTCAAGCAGATCGTTCAAACATTTG |
| ID | Terminator | GCAATAAAGTTTCTTAAGATTGAAT |
| NO: 76 | | CCTGTTGCCGGTCTTGCGATGATTA |
| | | TCATATAATTTCTGTTGAATTACGT |
| | | TAAGCATGTAATAATTAACATGTAA |
| | | TGCATGACGTTATTTATGAGATGGG |
| | | TTTTTATGATTAGAGTCCCGCAATT |
| | | ATACATTTAATACGCGATAGAAAAC |
| | | AAAATATAGCGCGCAAACTAGGATA |
| | | AATTATCGCGCGCGGTGTCATCTAT |
| | | GTTACTAGATCGA |
| SEQ | β-casein | AGAGAACTTGAAGAATTGAATGTTC |
| ID | codon | CAGGAGAGATTGTTGAAAGTCTCTC |
| NO: 77 | optimized | CTCTTCTGAGGAAAGCATCACAAGA |
| | for N. | ATCAACAAGAAAATTGAGAAGTTTC |
| | benthamiana | AGAGTGAAGAGCAACAACAGACTGA |
| | | AGATGAATTACAAGATAAGATTCAT |
| | | CCTTTTGCTCAAACACAGTCACTTG |
| | | TGTATCCATTCCCTGGACCAATTCC |
| | | AAATTCTTTACCACAAAACATTCCT |
| | | CCTCTGACTCAAACTCCTGTCGTTG |
| | | TTCCTCCGTTCTTGCAACCAGAAGT |
| | | TATGGGAGTTTCAAAGGTTAAAGAA |
| | | GCAATGGCTCCAAAGCATAAAGAGA |
| | | TGCCATTCCCAAAATACCCTGTGGA |
| | | GCCTTTCACAGAATCTCAAAGCTTG |
| | | ACTCTCACTGATGTTGAGAATCTTC |
| | | ATTTGCCTCTTCCATTGCTTCAATC |
| | | ATGGATGCATCAACCTCATCAGCCT |
| | | TTGCCACCAACAGTGATGTTTCCAC |
| | | CTCAATCTGTTCTCTCTCTTTCTCA |
| | | GTCTAAAGTTCTTCCGGTTCCGCAG |
| | | AAAGCTGTGCCTTATCCTCAGAGAG |
| | | ATATGCCTATTCAAGCTTTTCTTCT |
| | | CTACCAAGAACCAGTTTTGGGTCCT |
| | | GTTCGTGGTCCATTTCCCATCATAG |
| | | TT |
| SEQ | κ-casein | CAGGAGCAAAACCAAGAGCAACCTA |
| ID | codon | TTCGTTGTGAAAAAGACGACGTTTT |
| NO: 78 | optimized | AAGTCGATATCCGAGCTACGGTCTT |
| | for N. | AATTACTATCAACAGAAGCCAGTTG |
| | benthamiana | CCTTGATAAACAACCAGTTTCTCCC |
| | | ATATCCCTACTATGCTAAGCCGGCC |
| | | GCCGTCCGTTCTCCTGCTCAGATAT |
| | | TGCAGTGGCAGGTTTTATCCAACAC |
| | | TGTCCCGGCGAAGTCTTGCCAGGCG |
| | | CAACCCACCACCATGGCTAGACACC |
| | | CGCATCCGCACCTTTCCTTTATGGC |
| | | TATTCCTCCCAAAAAGAACCAAGAC |
| | | AAGACAGAGATCCCGACCATAAATA |
| | | CTATCGCTTCTGGTGAGCCAACTTC |
| | | AACACCGACTATTGAAGCGGTGGAG |
| | | AGCACGGTCGCCACACTTGAAGCAT |
| | | CCCCAGAGGTGACTGAATCACCCCC |
| | | GGAGATAAACACGGTACAGGTCACC |
| | | TCAACGGCTGTT |
| SEQ | α-casein | CAGGAGCAAAACCAAGAGCAACCTA |
| ID | codon | TTCGTTGTGAAAAAGACGACGTTTT |
| NO: 79 | optimized | AAGTCGATATCCGAGCTACGGTCTT |
| | for N. | AATTACTATCAACAGAAGCCAGTTG |
| | benthamiana | CCTTGATAAACAACCAGTTTCTCCC |
| | | ATATCCCTACTATGCTAAGCCGGCC |
| | | GCCGTCCGTTCTCCTGCTCAGATAT |
| | | TGCAGTGGCAGGTTTTATCCAACAC |
| | | TGTCCCGGCGAAGTCTTGCCAGGCG |
| | | CAACCCACCACCATGGCTAGACACC |
| | | CGCATCCGCACCTTTCCTTTATGGC |
| | | TATTCCTCCCAAAAAGAACCAAGAC |
| | | AAGACAGAGATCCCGACCATAAATA |
| | | CTATCGCTTCTGGTGAGCCAACTTC |
| | | AACACCGACTATTGAAGCGGTGGAG |
| | | AGCACGGTCGCCACACTTGAAGCAT |
| | | CCCCAGAGGTGACTGAATCACCCCC |
| | | GGAGATAAACACGGTACAGGTCACC |
| | | TCAACGGCTGTT |
| SEQ | Protein | MANKLFLVCATFALCFLLTNARPKH |
| ID | produced by | PIKHQGLPQEVLNENLLRFFVAPFP |
| NO: 80 | pMOZ701 | EVFGKEKVNELSKDIGSESTEDQAM |
| | (aSI-HA- | EDIKQMEAESISSSEEIVPNSVEQK |
| | 6His- | HIQKEDVPSERYLGYLEQLLRLKKY |
| | HDEL) | KVPQLEIVPNSAEERLHSMKEGIHA |
| | | QQKEPMIGVNQELAYFYPELFRQFY |
| | | QLDAYPSGAWYYVPLGTQYTDAPSF |
| | | SDIPNPIGSENSEKTTMPLWSSYPY |
| | | DVPDYAHHHHHHHDEL |
| SEQ | Protein | MANKLFLVCATFALCFLLTNARELE |
| ID | produced by | ELNVPGEIVESLSSSEESITRINKK |
| NO: 81 | pMOZ702 | IEKFQSEEQQQTEDELQDKIHPFAQ |
| | (β-HA- | TQSLVYPFPGPIPNSLPQNIPPLTQ |
| | 6His- | TPVVVPPFLQPEVMGVSKVKEAMAP |
| | HDEL) | KHKEMPFPKYPVEPFTESQSLTLTD |
| | | VENLHLPLPLLQSWMHQPHQPLPPT |
| | | VMFPPQSVLSLSQSKVLPVPQKAVP |
| | | YPQRDMPIQAFLLYQEPVLGPVRGP |
| | | FPIIVSYPYDVPDYAHHHHHHHDEL |
| SEQ | Protein | MANKLFLVCATFALCFLLTNAQEQN |
| ID | produced by | QEQPIRCERDERFFSDKJAKYIPIQ |
| NO: 82 | pMOZ702 | YVLSRYPSYGLNYYQQKPVALINNO |
| | (κ-HA- | FLPYPYYAKPAAVRSPAQILQWQVL |
| | 6His- | SNTVPAKSCQAQPTTMARHPHPHLS |
| | HDEL) | FMAIPPKKNQDKTEIPTINTIASGE |
| | | PTSTPTIEAVESTVATLEASPEVTE |
| | | SPPEINTVQVTSTAVSYPYDVPDYA |
| | | HHHHHHHDEL |
| SEQ | Cow | MLLPKLERSAARPSGEPGCSCAQPA |
| ID | Fam20C | AEAAAPGWAQARGHPGGELEAAASA |
| NO: 83 | | AGDAGWPNKHTLRILQDFSSDPSSN |
| | | LTSHSLEKLPPAAEAAEGAPPGQDP |
| | | GVRRPPDPAHRPLPRDPGPRGPVLP |
| | | PGLSGDGSLLTRLFQHPLYQVPIPP |
| | | LTEGDVLFNVNSDIRFNPKAATAEN |
| | | PDWPHEGPEDEFLPTGEAAVDSYPN |
| | | WLKFHIGINRYELYSRHNPAVGALL |
| | | QDLGTQKITSVAMKSGGTQLKLIMT |
| | | FONYGQALFKPMKQTREQETPPDFF |
| | | YFSDYERHNAEIAAFHLDRILDFRR |
| | | VPPVAGRLVNMTKEIRDVTRDKKLW |
| | | RTFFISPANNVCFYGECSYYCSTEH |
| | | ALCGKPDQIEGSLAAFLPDLALAKR |
| | | KTWRNPWRRSYHKRKKAEWEVDPDY |
| | | CEEVRQTPPYDSSHRLLDVMDMTIF |
| | | DFLMGNMDRHHYETFEKFGNETFII |
| | | HLDNGRGFGKHSHDELSILVPLQQC |
| | | CRIRRSTYLRLQLLAQEEHRLSLLM |
| | | AEALRADRVAPVLFQPHLEALDRRL |
| | | RIVLRAVGDCVEKDGLHSVVEDDLG |
| | | PEHRAAAGR |
| SEQ | Cow | MHVSSHLGPRVRSRLQPRERPLGCP |
| ID | Fam20A | CARRAASPAPGPAPSAPRRVEPSGG |
| NO: 84 | | GDPGSKLRALFAHPLYNEPEEPPLL |
| | | GPEDSLLAGPEALRYYRRKVARWNR |
| | | RRKMYKEQLNLTSPEPPVQLRQEAS |
| | | WVQFHLGINRHGLYPRSSPVVSKLL |
| | | QDMRHFPTISADYSQDEKALLGACD |
| | | CSQIVKPSGVHLKLVLRFSDFGKAM |
| | | FKPMROQRDEETPEDFFYFIDFORH |
| | | NAEIAAFHLDRILDFRRVPPTVGRL |
| | | VNVTREILEVTRNEILQSVFFVSPA |
| | | NNVCFFAKCPYMCKTEYAVCGSPHL |
| | | LEGSLSAFLPSLNLAPRFSMPSPWI |
| | | RSYSLAGREEWEVNPLYCEAVKQAY |
| | | PHNSSSRLLNIIDMAIFDFLIGNMD |
| | | RHHYEMFTKFGEDGFLIHLDNARGF |
| | | GRHSHDEVSILAPLFQCCRIKRKTL |
| | | LHLQLLAQADYRLSDVMRESLLEDQ |
| | | LSPVLTEPHLLALDRRLOTVLRTVQ |
| | | DCIEAHGEQSVVADGPVGQWAPDSS |
| | | RANATS |

In some aspects, the current disclosure also provides methods for expressing a phosphorylated payload protein in a plant, comprising transforming the plant with a vector as described herein, and growing the transformed plant, wherein the payload protein is phosphorylated by the first or second kinase. In some instances, phosphorylation using the methods described herein leads to a higher yield or improved quality of food protein production in plants, compared to using an alternative method that does not use vectors described herein.

In some aspects, the current disclosure also provides methods of expressing a phosphorylated payload protein in a plant, comprising transforming the plant with a first vector, a second vector, and a third vector; and growing the transformed plant, wherein the payload protein is phosphorylated by the kinase; wherein the first vector comprises a first polynucleotide sequence encoding a first kinase, the second vector comprises a second polynucleotide sequence encoding a second kinase, and the third vector comprises a third polynucleotide sequence encoding the payload protein.

In some aspects, the current disclosure also provides food products and food product substitutes comprising the phosphorylated payload protein made using the method describe above. Contemplated food products include dairy products or products that resembles a dairy product (i.e., dairy product substitutes). The term “dairy product” as used herein refers to milk (e.g., whole milk (at least 3.25% milk fat), partly skimmed milk (from 1% to 2% milk fat), skim milk (less than 0.2% milk fat), cooking milk, condensed milk, flavored milk, goat milk, sheep milk, dried milk, evaporated milk, milk foam), and products derived from milk, including but not limited to yogurt (e.g., whole milk yogurt (at least 6 grams of fat per 170 g), low-fat yogurt (between 2 and 5 grams of fat per 170 g), nonfat yogurt (0.5 grams or less of fat per 170 g), greek yogurt (strained yogurt with whey removed), whipped yogurt, goat milk yogurt, Labneh (labne), sheep milk yogurt, yogurt drinks (e.g., whole milk Kefir, low-fat milk Kefir), Lassi), cheese (e.g., whey cheese such as ricotta; pasta filata cheese such as mozzarella; semi-soft cheese such as Havarti and Muenster; medium-hard cheese such as Swiss and Jarlsberg; hard cheese such as Cheddar and Parmesan; washed curd cheese such as Colby and Monterey Jack; soft ripened cheese such as Brie and Camembert; fresh cheese such as cottage cheese, feta cheese, cream cheese, and curd; processed cheese; processed cheese food; processed cheese product; processed cheese spread; enzyme-modulated cheese; cold-pack cheese), dairy-based sauces (e.g., fresh, frozen, refrigerated, or shelf stable), dairy spreads (e.g., low-fat spread, low-fat butter), cream (e.g., dry cream, heavy cream, light cream, whipping cream, half-and-half, coffee whitener, coffee creamer, sour cream, creme fraiche), frozen confections (e.g., ice cream, smoothie, milk shake, frozen yogurt, sundae, gelato, custard), dairy desserts (e.g., fresh, refrigerated, or frozen), butter (e.g., whipped butter, cultured butter), dairy powders (e.g., whole milk powder, skim milk powder, fat-filled milk powder (i.e., milk powder comprising plant fat in place of all or some animal fat), infant formula, milk protein concentrate (i.e., protein content of at least 80% by weight), milk protein isolate (i.e., protein content of at least 90% by weight), whey protein concentrate, whey protein isolate, demineralized whey protein concentrate, demineralized whey protein concentrate, .beta.-lactoglobulin concentrate, .beta.-lactoglobulin isolate, .alpha.-lactalbumin concentrate, .alpha.-lactalbumin isolate, glycomacropeptide concentrate, glycomacropeptide isolate, casein concentrate, casein isolate, nutritional supplements, texturizing blends, flavoring blends, coloring blends), ready-to-drink or ready-to-mix products (e.g., fresh, refrigerated, or shelf stable dairy protein beverages, weight loss beverages, nutritional beverages, sports recovery beverages, and energy drinks), puddings, gels, chewables, crisps, and bars. As used herein, the term “food product substitute” (e.g., “dairy product substitute”) refers to a food product that resembles a conventional food product (e.g., can be used in place of the conventional food product). Such resemblance can be due to any physical, chemical, or functional attribute. In some embodiments, the resemblance of the food product provided herein to a conventional food product is due to a physical attribute. Non-limiting examples of physical attributes include color, shape, mechanical characteristics (e.g., hardness, G′ storage modulus value, shape retention, cohesion, texture (i.e., mechanical characteristics that are correlated with sensory perceptions (e.g., mouthfeel, fattiness, creaminess, homogenization, richness, smoothness, thickness), viscosity, and crystallinity. In some embodiments, the resemblance of the food product provided herein and a conventional food product is due to a chemical/biological attribute. Non-limiting examples of chemical attributes include nutrient content (e.g., type and/or amount of amino acids (e.g., PDCAAS score), type and/or amount of lipids, type and/or amount of carbohydrates, type and/or amount of minerals, type and/or amount of vitamins), pH, digestibility, shelf-life, hunger and/or satiety regulation, taste, and aroma. In some embodiments, the resemblance of the food product provided herein to a conventional food product is due to a functional attribute. Non-limiting examples of functional attributes include gelling/agglutination behavior (e.g., gelling capacity (i.e., time required to form a gel (i.e., a protein network with spaces filled with solvent linked by hydrogen bonds to the protein molecules) of maximal strength in response to a physical and/or chemical condition (e.g., agitation, temperature, pH, ionic strength, protein concentration, sugar concentration, ionic strength)), agglutination capacity (i.e., capacity to form a precipitate (i.e., a tight protein network based on strong interactions between protein molecules and exclusion of solvent) in response to a physical and/or chemical condition), gel strength (i.e., strength of gel formed, measured in force/unit area (e.g., pascal (Pa))), water holding capacity upon gelling, syneresis upon gelling (i.e., water weeping over time)), foaming behavior (e.g., foaming capacity (i.e., amount of air held in response to a physical and/or chemical condition), foam stability (i.e., half-life of foam formed in response to a physical and/or chemical condition), foam seep), thickening capacity, use versatility (i.e., ability to use the food product in a variety of manners and/or to derive a diversity of other compositions from the food product; e.g., ability to produce food products that resemble milk derivative products such as yoghurt, cheese, cream, and butter), and ability to form protein dimers.

In some aspects, the current disclosure also provides plants transformed with a vector as described herein, wherein the payload protein is phosphorylated by the first or the second kinase in vivo in the plant. Contemplated plants can be a dicot plant, for example, Arabidopsis, tobacco, tomato, potato, sweet potato, cassava, alfalfa, lima bean, pea, chick pea, soybean, carrot, strawberry, lettuce, oak, maple, walnut, rose, mint, squash, daisy, quinoa, buckwheat, mung bean, cow pea, lentil, lupin, peanut, fava bean, French beans, mustard, and cactus. Contemplated plants can also be a monocot plant, for example, turf grass, com, rice, oat, wheat, barley, sorghum, orchid, iris, lily, onion, palm, and duckweed.

Described herein, in some aspects, are vectors for expressing a phosphorylated casein protein in a plant. For example, a vector can comprise polynucleotide sequences encoding a κ-casein, and at least one of αS1-casein, αS2-casein, and β-casein. In some instances, the casein protein has at least 50%, 60%, 70%, 80%, 85%, 90%, 95%, or 99% sequence identity to the amino acid SEQ ID NO:36, SEQ ID NO: 37, SEQ ID NO:38, SEQ ID NO:39, SEQ ID NO:77, SEQ ID NO:78, SEQ ID NO:79, SEQ ID NO:80, SEQ ID NO:81, or SEQ ID NO:82.

In some aspects, the current disclosure also provides methods of enhancing casein micelle formation in a plant, comprising transforming the plant with a vector as described herein and growing the transformed plant, wherein at least one of a κ-casein, and αS1-casein, αS2-casein, and β-casein.

In some aspects, the current disclosure also provides methods of enhancing casein micelle formation in a plant, comprising transforming the plant with a first vector and a second vector, and growing the transformed plant; wherein the first vector comprises a first polynucleotide sequence encoding a kinase, wherein the second vector comprises a second polynucleotide sequence encoding a κ-casein, and at least one of αS1-casein, αS2-casein, and β-casein; wherein at least one of a κ-casein, and αS1-casein, αS2-casein, and β-casein is phosphorylated by the kinase, and wherein the κ-casein and at least one of αS1-casein, αS2-casein, and β-casein form the casein micelle in the plant in vivo.

In some aspects, the current disclosure also provides methods of enhancing casein micelle formation in a plant, comprising transforming the plant with a first vector, a second vector, and a third vector, and growing the transformed plant; wherein the first vector comprises a first polynucleotide sequence encoding a kinase, wherein the second vector comprises a second polynucleotide sequence encoding a κ-casein, wherein the third vector comprises a third polynucleotide sequence encoding at least one of αS1-casein, αS2-casein, and β-casein wherein at least one of a κ-casein, and αS1-casein, αS2-casein, and β-casein is phosphorylated by the kinase, and wherein the κ-casein and at least one of αS1-casein, αS2-casein, and β-casein form the casein micelle in the plant in vivo.

In some aspects, phosphorylation using the methods described herein leads to improved micelle formation in plant cells, for example, in terms of increased number of micelles, micelles becoming more stable, and increased solubility of casein proteins. As a result, food products containing phosphorylated caseins made using the methods described herein have superior quality, including, for example, increased viscosity, melting point, and binding to calcium (e.g., calcium phosphate) than food products without phosphorylated caseins.

In some aspects, phosphorylation of a casein protein in a plant by using the vectors and methods described herein increases the expression level of the casein protein in the plant, wherein the casein protein is selected form the group consisting of κ-casein, αS1-casein, αS2-casein, and β-casein, and wherein phosphorylation of a casein protein increases expression level of the casein protein by at least 10%, 20%, 30%, 40%, 50%, 60%, 70%, 80%, 90%, or even 100%.

In some aspects, phosphorylation of a casein protein in a plant by using the vectors and methods described herein increases its ability to aggregate or bind to another casein protein, wherein the casein protein is selected form the group consisting of κ-casein, αS1-casein, αS2-casein, and β-casein. In some aspects, phosphorylation of a casein protein in a plant by using the vectors and methods described herein improves casein micelle formation, by increasing the number of micelles, or by stabilizing the micelles, or both. In some aspects, phosphorylation of a casein protein in a plant by using the vectors and methods described herein increases its binding to calcium by at least 10%, 20%, 30%, 40%, 50%, 60%, 70%, 80%, 90%, or even 100%.

In some aspects, phosphorylation of a casein protein in a plant by using the vectors and methods described herein increases the viscosity of a liquid containing the phosphorylated casein proteins, compared to a solution containing same amount of unphosphorylated casein proteins.

In some aspects, the current disclosure also provides a plant cell co-expressing at least one casein protein and at least one kinase. In some cases, the at least one casein protein comprises at least one of κ-casein, αS1-casein, αS2-casein, and β-casein. In some cases, the at least one casein protein comprises κ-casein and at least one of αS1-casein, αS2-casein, and B-casein. In some cases, the at least one kinase is a mammalian kinase. In some cases, the at least one kinase comprises two different kinases. In some cases, the at least one kinase is at least one of FAM20A, FAM20C, or human Casein kinase 2 (CK2), or any combination thereof. In some cases, the at least one kinase has at least 80% sequence identity to SEQ ID NO: 40, SEQ ID NO: 41, SEQ ID NO: 43, SEQ ID NO: 83, or SEQ ID NO: 84.

In some cases, the plant cell is co-transformed with one or more plasmids comprising polynucleotide sequences encoding at least one casein protein and at least one kinase. In some cases, the polynucleotide sequences encoding the at least one casein protein and the at least one kinase are in the same plasmid. In some cases, the polynucleotide sequences encoding the at least one casein protein and the at least one kinase are in different plasmids. In some cases, the at least one casein protein comprises at least one of κ-casein, αS1-casein, αS2-casein, and β-casein and wherein the polynucleotide sequences encoding different casein proteins are in different plasmids.

In some aspects, the current disclosure also provides a plant cell genetically modified to increase free phosphate inside the plant cell. In some cases, the plant cell co-expresses 1) at least one casein protein, 2) at least one kinase, and 3) 3-phytase increase free phosphate inside the plant cell. In some cases, the plant cell co-expresses 1) at least one casein protein, 2) at least one kinase, and 3) purple acid phosphatase increase free phosphate inside the plant cell.

In some aspects, the current disclosure also provides a plant cell genetically modified to increase free calcium in the plant cell. In some cases, the plant cell co-expresses 1) at least one casein protein, 2) at least one kinase, and 3) oxalate decarboxylase to increase free calcium in the plant cell. In some cases, the plant cell co-expressing at least one casein protein and at least one kinase has oxalyl-CoA synthetase gene knocked-out or under-expressed to increase free calcium in the plant cell.

In some cases, the plant cell is genetically modified to increase free phosphate and free calcium inside the plant cell. In some cases, the plant cell co-expresses 1) at least one casein protein, 2) at least one kinase, and 3) at least one of 3-phytase, a purple acid phosphatase, oxalate decarboxylase, or any combination thereof.

In some cases, the 3-phytase has at least 50%, 60%, 70%, 80%, 85%, 90%, 95%, or 99% sequence identity to SEQ ID NO: 85, SEQ ID NO: 86, or SEQ ID NO: 87. In some cases, the purple acid phosphatase has at least 50%, 60%, 70%, 80%, 85%, 90%, 95%, or 99% sequence identity to SEQ ID NO: 88 or SEQ ID NO: 89. In some cases, the oxalate decarboxylase has at least 50%, 60%, 70%, 80%, 85%, 90%, 95%, or 99% sequence identity to SEQ ID NO: 90, SEQ ID NO: 91, or SEQ ID NO: 92. In some cases, the oxalyl-CoA synthetase has at least 50%, 60%, 70%, 80%, 85%, 90%, 95%, or 99% sequence identity to SEQ ID NO: 93, SEQ ID NO: 94, or SEQ ID NO: 95.

Examples certain genes that can modified to increase free calcium or free phosphate are listed in Table 2.

TABLE 2
Examples of genes that can be modified in a plant cell that
co-expresses at least one kinase and at least one casein
protein, to increase free calcium or free phosphate.

| Protein | | | SEQ |
|---|---|---|---|
| Family | Specific Protein | Source Species | ID NO: |
| 3-Phytase | 168phyA | Bacillus subtilus | 85 |
| | 3-phytase A | Aspergillus niger | 86 |
| | 3-phytase B | Aspergillus niger | 87 |
| Purple Acid | PAP15 | Arabidopsis thaliana | 88 |
| Phosphatase | PAP3 | Glycine max | 89 |
| Oxalate | odxC | Bacillus subtilis | 90 |
| decarboxylase | ABL_03746 | Aspergillus niger | 91 |
| | OXDC | Flammulina velutipes | 92 |
| oxalyl-CoA | AAE3 | Arabidopsis thaliana | 93 |
| synthetase | GLYMA_11G198300 | Glycine max | 94 |
| | Oxalyl-CoA | Glycine max | 95 |
| | synthetase | | |

In some aspects, the current disclosure also provides a plant cell disclosed herein having Inositol-3-phosphate synthase gene (for example, soybean Inositol-3-phosphate synthase, SEQ ID NO: 94) knocked-out or under-expressed in the plant cell, which can be achieved by RNAi, CRISPR-Cas9, or other suitable genome editing systems.

In some aspects, the current disclosure also provides methods of producing a casein micelle, comprising growing a plant comprising a plant cell disclosed herein, wherein the at least one casein protein comprises κ-casein and at least one of αS1-casein, αS2-casein, or β-casein wherein the at least one casein protein is phosphorylated by the at least one kinase in vivo, and the κ-casein and at least one of αS1-casein, αS2-casein, or β-casein form a casein micelle in vivo; and collecting the casein micelle from the plant.

In some aspects, the current disclosure also provides methods of producing a micelle, comprising mixing phosphorylated casein proteins in a liquid to form at least one casein micelle, wherein the casein proteins comprises κ-casein and at least one of αS1-casein, αS2-casein, and β-casein, wherein one or more casein proteins are phosphorylated. In some cases, the one or more casein proteins are expressed in different plants of the same species. In some cases, the one or more casein proteins are expressed in different species of plants. In some cases, the same plant produce the one or more casein proteins.

In some cases, the method further comprises adding a salt or phosphate acid to the liquid. The salt comprises at least one of a phosphate salt or a calcium salt (e.g., calcium chloride (CaCl₂). Contemplated phosphate salts include a salt having a phosphate group including dihydrogen phosphate, hydrogen phosphate, or phosphate, for example, sodium phosphate.

In some aspects, the current disclosure also provides food product or food product substitute, comprising a phosphorylated casein protein produced by the plant cell disclosed herein. In some aspects, the food product or food product substitute comprises a product traditionally derived from milk, comprising at least one of yogurt, low-fat yogurt, nonfat yogurt, greek yogurt, whipped yogurt, goat milk yogurt, Labneh (labne), sheep milk yogurt, yogurt drink, Lassi, cheese, dairy-based sauce, dairy spread, cream, frozen confections, dairy desserts, butter, dairy powders, infant formula, milk protein concentrate, milk protein isolate, milk protein concentrate, whey protein isolate, demineralized whey protein concentrate, demineralized whey protein concentrate, beta-lactoglobulin concentrate, beta-lactoglobulin isolate, alpha-lactalbumin concentrate, alpha-lactalbumin isolate, glycomacropeptide concentrate, glycomacropeptide isolate, casein concentrate, casein isolate, nutritional supplements, ready-to-drink or ready-to-mix product, pudding, gel, chewable, crisp, and bar.

### BRIEF DESCRIPTION OF THE DRAWINGS

FIG. 1 is an example of a flow for forming in vivo casein micelles in an embodiment.

FIG. 2 is an example of a schematic illustration of a plasmid used in FIG. 1.

FIG. 3A is an example with additional details from the plant transformation to the post-translation modification.

FIG. 3B is an example with additional details for the in vivo formation.

FIG. 3C is an example of a schematic illustration of a transcription of proteins which impart herbicide resistance to the transformed plant.

FIG. 3D is an example of a schematic illustration of suppression of native seed storage proteins by RNAi transcribed by a portion of the plasmid of FIG. 1.

FIG. 3E is an example of a schematic illustration of a transcription of a portion of the plasmid of FIG. 1 and resulting proteins used to increase calcium concentrations in the plant cell.

FIG. 3F is an example of a schematic illustration of a transcription of a portion of the plasmid of FIG. 1 and resulting proteins used to increase phosphate concentrations in the plant cell.

FIG. 3G is an example with further additional details of the in vivo formation.

FIG. 4 is an example of a schematic illustration of a portion of a plasmid in Arabidopsis.

FIG. 5 is an example of a schematic illustration of a portion of a plasmid in Arabidopsis for a screenable marker in plants.

FIG. 6 is an example of a schematic illustration of a portion of a plasmid in soybean.

FIG. 7 is an example of a schematic illustration of a portion of a plasmid in soybean for herbicide resistance in plants.

FIG. 8 is an example of a schematic illustration of a portion of the plasmid of FIG. 1 for soybean for suppression of native seed storage proteins in plants.

FIG. 9 is an example of a schematic illustration of a portion of a plasmid for soybean to regulate intracellular concentrations of minerals which can enhance micelle formation.

FIG. 10 is an example of a flow for the purification of micelles formed in vivo in soybean.

FIG. 11 shows an example of an expression plasmid for the production of kinase proteins in plants.

FIG. 12 shows a map of a pMOZ12 expression plasmid, which is an example expression plasmid for the production of recombinant H. sapiens Fam20A in plants.

FIG. 13 shows a map of a pMOZ702 expression plasmid, which is an example expression plasmid for the production of recombinant B. taurus Beta Casein in plants.

FIG. 14 shows a map of a pMOZ14 expression plasmid, which is an example expression plasmid for the production of recombinant B. taurus Fam20C in plants.

FIG. 15 shows a map of a pMOZ15 expression plasmid, which is an example expression plasmid for the production of recombinant B. taurus Fam20A in plants.

FIG. 16 shows a map of a pMOZ700 expression plasmid, which is an example expression plasmid for the production of recombinant B. taurus Kappa Casein in plants.

FIG. 17 shows a map of a pMOZ701 expression plasmid, which is an example expression plasmid for the production of recombinant B. taurus αS1 Casein in plants.

FIG. 18 shows a map of a vector backbone including a nucleic acid sequence encoding a β-lactamase.

FIG. 19 shows results of Western blot showing the expression of recombinant Beta casein, HsFam20C, and HsFam20A in a systemically-infected N. benthamiana plant using combinations of the pMOZ11 (expresses HsFam20C), pMOZ12 (expresses HsFam20A) and pMOZ702 (expresses beta casein) expression plasmids.

FIG. 20 shows results of Western blot showing the expression of recombinant Beta casein, HsFam20C, and HsFam20A in a systemically-infected N. benthamiana plant using combinations of the pMOZ11 (expresses HsFam20C), pMOZ12 (expresses HsFam20A), pMOZ14 (expresses BtFam20C), pMOZ15 (expresses BtFam20A) and pMOZ702 (expresses beta casein) expression plasmids.

FIG. 21 shows a map of a pMOZ401 expression plasmid, which is an example expression plasmid for the production of recombinant B. taurus Beta Casein in plants.

FIG. 22 shows a map of a pMOZ882 expression plasmid, which is an example expression plasmid for the production of recombinant B. taurus Beta Casein in plants.

### DETAILED DESCRIPTION

The following embodiments are described in sufficient detail to enable those skilled in the art to make and use the invention. It is to be understood that other embodiments would be evident based on the present disclosure, and that system, process, or mechanical changes may be made without departing from the scope of an embodiment of the present disclosure.

In the following description, numerous specific details are given to provide a thorough understanding of the invention. However, it will be apparent that the invention may be practiced without these specific details. In order to avoid obscuring an embodiment of the present disclosure, some well-known techniques, system configurations, and process steps are not disclosed in detail.

The drawings showing embodiments of the system are semi-diagrammatic, and not to scale and, particularly, some of the dimensions are for the clarity of presentation and are shown exaggerated in the drawing figures. Similarly, although the views in the drawings for ease of description generally show similar orientations, this depiction in the figures is arbitrary for the most part. Generally, the invention can be operated in any orientation.

The term “invention” or “present invention” as used herein is not meant to be limiting to any one specific embodiment of the invention but applies generally to any and all embodiments of the invention as described in the claims and specification.

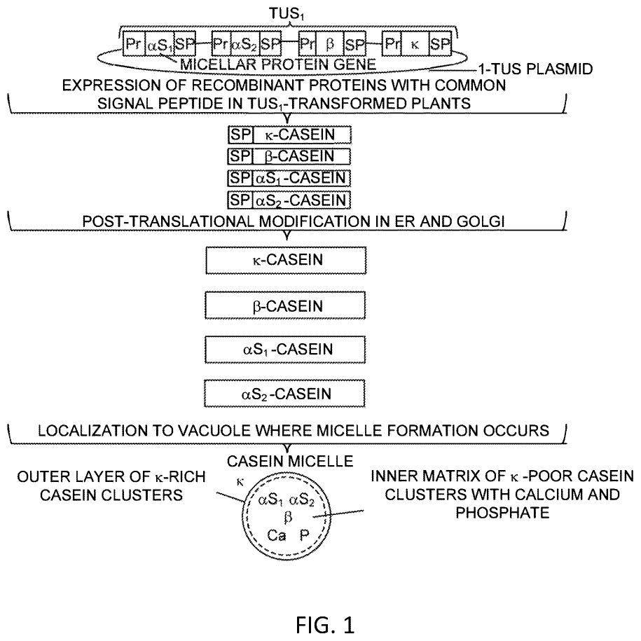

Referring now to FIG. 1, therein is shown an example of a flow for forming in vivo casein micelles in an embodiment. In this example, FIG. 1 depicts the flow for forming the casein micelles by a plant transformation, a recombinant casein protein formation, a post-translation modification, and an in-vivo formation. As a specific example, FIG. 1 is a schematic illustration of the elements of a plasmid of this embodiment and its use in creation of micelles in vivo in a plant cell.

In this example for the plant transformation, a plant is transformed using a plasmid including a single transcription unit set. As used herein “plasmid” is a deoxyribonucleic acid (DNA) molecule capable of replication in a host cell and to which another DNA segment can be operatively linked so as to bring about replication of the attached DNA segment. As it relates to this example, methods for plant transformation include microprojectile bombardment as illustrated in U.S. Pat. Nos. 5,015,580; 5,550,318; 5,538,880; 6,153,812; 6,160,208; 6,288,312 and 6,399,861, all of which are incorporated herein by reference. Methods for plant transformation also include Agrobacterium-mediated transformation as illustrated in U.S. Pat. Nos. 5,159,135; 5,824,877; 5,591,616 and 6,384,301, all of which are incorporated herein by reference. Recipient cells for the plant transformation include, but are not limited to, meristem cells, callus, immature embryos, hypocotyls explants, cotyledon explants, leaf explants, and gametic cells such as microspores, pollen, sperm and egg cells, and any cell from which a fertile plant may be regenerated, as described in U.S. Pat. Nos. 6,194,636; 6,232,526; 6,541,682 and 6,603,061 and U.S. Patent Application publication US 2004/0216189 A1, all of which are incorporated herein by reference.

Continuing this example for the plant transformation, the plasmid including the single transcription unit set is shown and abbreviated in FIG. 1 as 1-TUS PLASMID. The transcription unit set included on this plasmid is transcription unit set 1 shown and abbreviated in FIG. 1 as TUS₁. As used herein “transcription unit set” is a segment of DNA including one or more transcription units. The purpose of a transcription unit set includes but is not limited to protein expression, gene suppression, regulatory ribonucleic acid (RNA) production, and herbicide resistance. As used herein “transcription unit” is a segment of DNA including at least a promoter DNA and transcribable DNA. As used herein “promoter” means regulatory DNA for initiating RNA transcription. A “plant promoter” is a promoter capable of initiating transcription in plant cells whether or not its origin is a plant cell. As used herein “terminator” means any DNA sequence that causes RNA transcription to terminate.

Further continuing this example for the plant transformation shown in FIG. 1 as an embodiment, the transcription unit set 1 includes four segments of DNA; each encoding RNA for one of the four proteins found in a casein micelle: an αS₁ casein, an αS₂ casein, a β casein, and a κ casein. For clarity and as an example, the genes encoding the αS₁ casein, the αS₂ casein, the β casein, and the κ casein are shown and abbreviated in FIG. 1 as αS₁, as αS₂, as 3, and as κ, respectively, and shown and annotated in FIG. 1 as MICELLAR PROTEIN GENE. Each DNA segment encoding RNA for one of the four proteins found in a casein micelle is operably linked to a promoter, shown and abbreviated in FIG. 1 as P, and includes a plant-derived, tissue specific, N-terminal signal peptide, shown and abbreviated in FIG. 1 as SP. As used herein “operably linked” is the association of two or more DNA fragments in a DNA construct such that the function of one is controlled by the other, for example DNA encoding a protein associated with DNA encoding a promoter. In some embodiments, the N-terminal signal peptide targets the recombinant casein proteins to the plant vacuoles. In other embodiments, the recombinant casein proteins are targeted to and retained in the endoplasmic reticulum.

As an example for the recombinant casein protein formation, when the four segments of DNA included in transcription unit set 1 are transcribed and translated in a transgenic plant (not shown), four recombinant casein proteins, each including a plant-derived tissue specific signal peptide, are formed in the cytoplasm of the plant cell. The recombinant casein proteins are shown and abbreviated in FIG. 1 as αS₁-CASEIN, as αS₂-CASEIN, as β-CASEIN, and as x-CASEIN, respectively, and are also referred to herein as “recombinant casein proteins” for brevity. As used herein, “transgenic” plant is a plant whose genome has been altered by the stable integration of recombinant DNA. As an example of stable integration, the transgenic plant includes a plant regenerated from an originally-transformed plant cell and progeny transgenic plants from later generations or crosses of a transformed plant. As used herein “recombinant DNA” refers to DNA which has been synthesized, assembled or constructed outside of a cell. Examples of recombinant DNA can include DNA containing naturally occurring DNA or complementary DNA (cDNA) or synthetic DNA.

As it relates to this example for the post-translation modification shown in FIG. 1 as an embodiment, the four recombinant casein proteins in the cytoplasm of the plant cell include the αS₁-casein, the αS₂-casein, the β-casein, and the κ-casein, each including a signal peptide (SP) that localizes the recombinant casein protein to specific organelles, for example the secretory pathway and protein storage vacuoles, in the plant cell. The signal peptide is removed from the recombinant casein proteins during post-translational modification that occurs in the endoplasmic reticulum and the Golgi apparatus of the plant cell. For clarity in this example, the endoplasmic reticulum and the Golgi apparatus are shown and abbreviated in FIG. 1 as ER, and as GOLGI, respectively. In this embodiment and example, phosphorylation occurs on the recombinant casein proteins prior to, during, or after migration to a specific tissue, shown in FIG. 1 as circles enclosing the letter “P” attached to each of the recombinant casein proteins. In other embodiments and examples, one or more post-translational modifications of the recombinant casein proteins can occur, including phosphorylation, glycosylation, ubiquitination, nitrosylation, methylation, acetylation, lipidation and proteolysis. In other embodiments no post-translational modifications occur on the recombinant casein proteins, or in other words, the post-translation modification is optional.

Continuing this example for the in vivo formation as an embodiment, an outer layer of the micelle is enriched in recombinant κ-casein shown and abbreviated in FIG. 1 as κ, and an inner matrix of the micelle includes the recombinant αS₁-casein, the recombinant αS₂-casein, the recombinant β-casein, the calcium and the phosphate, shown in and annotated in FIG. 1 as αS₁, αS₂, as αS, and β, respectively. Micelle formation is enhanced by the presence of intracellular calcium and phosphate, shown and abbreviated in FIG. 1 as Ca and P, respectively.

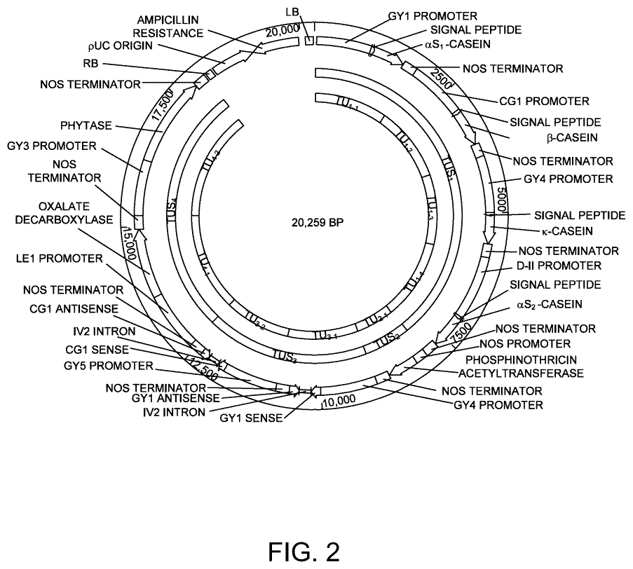

Referring now to FIG. 2, therein is shown an example of a schematic illustration of a plasmid used in FIG. 1. As a specific example, FIG. 2 is a schematic illustration of the elements of a plasmid of this embodiment.

In this example for the plant transformation of FIG. 1, an embodiment provides a plant that is transformed with one or more transfer DNAs including one or more transcription unit sets. As used herein “transfer DNA” (T-DNA) is DNA which integrates or is integrated into a genome.

For example, an Agrobacterium-mediated transformation T-DNA is part of a binary plasmid, which is flanked by T-DNA borders, and the binary plasmid is transferred into an Agrobacterium tumefaciens strain carrying a disarmed tumor inducing plasmid. Also for example, for a biolistic mediated transformation a gene gun is used for delivery of T-DNA, which is typically a biolistic construct containing promoter and terminator sequences, reporter genes, and border sequences or signaling peptides, to cells.

Continuing the example of a T-DNA used to transform a plant in an embodiment, the T-DNA includes four transcription unit sets: a transcription unit set 1, a transcription unit set 2, a transcription unit set 3, and a transcription unit set 4. For clarity, the transcription unit set 1, the transcription unit set 2, the transcription unit set 3, and the transcription unit set 4 are shown and abbreviated in FIG. 2 as TUS₁, as TUS₂, as TUS₃, and as TUS₄, respectively.

In this example as an embodiment, TUS₁ includes one transcription unit for each of the four casein proteins found in a casein micelle of FIG. 2: a transcription unit 1-1 includes DNA encoding αS₁-casein, a transcription unit 1-2 includes DNA encoding β-casein, a transcription unit 1-3 includes DNA encoding κ-casein, and a transcription unit 1-4 includes DNA encoding αS₁-casein. For clarity and brevity, the transcription unit 1-1, the transcription unit 1-2, the transcription unit 1-3, and the transcription unit 1-4 are shown and abbreviated in FIG. 2 as TU₁₋₁, as TU₁₋₂, as TU₁₋₃, and as TU₁₋₄, respectively. Each transcription unit in TUS₁ can also include DNA encoding the same plant-derived signal peptide. Additionally, each transcription unit in TUS₁ includes a promoter and a transcriptional terminator.

Continuing this example as an embodiment, TUS₂ includes one transcription unit, shown and abbreviated in FIG. 2 as TU₂₋₁, that includes a promoter, DNA encoding phosphinothricin acetyltransferase, and a transcriptional terminator. In other embodiments, TUS₂ can include one or more genes encoding a selectable marker that can impart herbicide or antibiotic resistance which enables the selection of transformed plants that produce micelles in vivo. Genes enabling selection of transformed plants include those conferring resistance to antibiotics, including as examples kanamycin, hygromycin B, gentamicin, and bleomycin.

Genes enabling selection of transformed plants also include those conferring resistance to herbicides, including as examples a glyphosate herbicide, a phosphinothricin herbicide, an oxynil herbicide, an imidazolinone herbicide, a dinitroaniline herbicide, a pyridine herbicide, a sulfonylurea herbicide, a bialaphos herbicide, a sulfonamide herbicide, and a glufosinate herbicide. Examples of such selectable markers are illustrated in U.S. Pat. Nos. 5,550,318; 5,633,435; 5,780,708 and 6,118,047, all of which are incorporated herein by reference. In other embodiments, TUS₂ includes one or more genes expressing a screenable marker which enables the visual identification of transformed plants that produce micelles in vivo. Genes expressing a screenable marker include genes encoding a colored or fluorescent protein, including as examples luciferase or green fluorescent protein (U.S. Pat. No. 5,491,084, herein incorporated by reference), and genes expressing β-glucuronidase or uidA gene (U.S. Pat. No. 5,599,670, herein incorporated by reference) for which various chromogenic substrates are known. In some embodiments, each of the genes encoding a selectable or screenable marker are operably linked to an inducible promoter, such as for example a NOS promoter, or a tissue-specific promoter, such as for example a promoter from the soybean a′ subunit of β-conglycinin, such that the translation of the selectable or screenable markers can be regulated.

Continuing this example as an embodiment, TUS₃ includes two transcription units that yield untranslated RNA molecules that suppress native seed protein gene translation. The first transcription unit in TUS₃, a transcription unit 3-1, includes the sense strand, or coding strand, of DNA encoding soybean Glycinin1, and the antisense strand, or non-coding strand, of DNA encoding soybean Glycinin1 separated by the potato IV2 intron. For clarity and brevity, the transcription unit 3-1 the sense strand or coding strand of DNA encoding soybean Glycinin1, and the antisense strand or non-coding strand of DNA encoding soybean Glycinin1, the potato IV2 intron are shown and annotated in FIG. 2 as TU₃₋₁ as GY1 SENSE, as GY1 ANTISENSE, and as IV2 INTRON, respectively. The second transcription unit in TUS₃, a transcription unit 3-2 includes the sense strand, or coding strand, of DNA encoding β-conglycinin 1 and the antisense strand, or non-coding strand, of DNA encoding β-conglycinin 1 separated by the potato IV2 intron. For clarity and brevity, the transcription unit 3-2, the sense strand or coding strand of DNA encoding β-conglycinin 1, the antisense strand or non-coding strand of DNA encoding β-conglycinin 1, and the potato IV2 intron are shown and annotated in FIG. 2 as TU₃. 2 as CG1 SENSE, as CG1 ANTISENSE, and as IV2 INTRON, respectively.

In other embodiments, TUS₃ includes other transcription units that yield untranslated RNA molecules that suppress native seed protein gene translation. As an example, in other embodiments, TUS₃ includes one transcription unit, a transcription unit 3-1, that includes a promoter from the soybean GY4 gene (SEQ ID NO:15), a miR319a microRNA from Arabidopsis thaliana that has been modified such that the homologous arms of the microRNA hairpin contain 21 nucleotide sequences matching a portion of the soybean GY1 gene sequence (SEQ ID NO:10), and a NOS transcriptional terminator (SEQ ID NO:35) (not shown).

Continuing this example as an embodiment, TUS₄ includes two transcription units that encode proteins which alter the intracellular environment in a manner that optimizes the production of micelles having requisite attributes including size, mineral content, protein content, protein distribution, and mass. The first transcription unit in TUS₄, a transcription unit 4-1 includes a promoter, DNA encoding oxalate decarboxylase, and a transcriptional terminator. For clarity and brevity, the transcription unit 4-1 is shown and abbreviated in FIG. 2 as TU₄₋₁. The second transcription unit in TUS₄, a transcription unit 4-2, includes a promoter, DNA encoding phytase, and a transcriptional terminator. For clarity and brevity, the transcription unit 4-2 is shown and abbreviated as TU₄₋₂. In this embodiment, transcription and translation of TU₄₋₁ yields an oxalate-degrading enzyme which increases the amount of free intracellular calcium available for capture and inclusion during micelle formation. Also in this embodiment, transcription and translation of TU₄₋₂ yields a phytase enzyme which increases the amount of free intracellular phosphate available for capture and inclusion during micelle formation. In some embodiments, each of the genes encoding oxalate-degrading enzymes or phytase enzymes are operably linked to a constitutive promoter, tissue specific promoter or an inducible promoter, such as for example, a nopaline synthase promoter or a promoter from the soybean β-conglycinin gene, such that the translation of proteins which alter the intracellular environment can be regulated. In some embodiments, TUS₄ includes both a transcription unit 4-1 that increases the intracellular calcium concentration and a transcription unit 4-2 that increases the intracellular phosphate concentration. In other embodiments, TUS₄ includes only a transcription unit 4-1 that increases the intracellular calcium concentration. In other embodiments, TUS₄ includes only a transcription unit 4-2 that increases the intracellular phosphate concentration.

In other embodiments, TUS₄ includes transcription units that increase the intracellular calcium concentration by expressing an oxalate oxidase enzyme (not shown). As an example, in other embodiments, TUS₄ includes one transcription unit, a transcription unit 4-1, that includes a promoter from the soybean GY4 gene (SEQ ID NO:15), the coding sequence for the oxalate oxidase 1 coding sequence from wheat that has been codon optimized for expression in soybean (SEQ ID NO:9), and the NOS transcriptional terminator (SEQ ID NO:35) (not shown). In other embodiments, TUS₄ includes transcription units that increase the intracellular phosphate concentration by suppressing the expression of the soybean myo-inositol-3-phosphate synthase (MIPS1) gene. As an example, in other embodiments, TUS₄ includes one transcription unit, a transcription unit 4-2, that includes a promoter from the soybean GY4 gene (SEQ ID NO:15), a portion of the MIPS1 coding sequence lacking a start codon (SEQ ID NO:21), the IV2 intron from potato (SEQ ID NO:25), the antisense of the MIPS1 sequence (SEQ ID NO:22), and the NOS transcriptional terminator (SEQ ID NO:35) (not shown).

In some embodiments of the disclosure, transcription unit sets are assembled in numeric order. In other embodiments, transcription unit sets can be assembled in any order.

In some embodiments of the disclosure, the plant is transformed with a plasmid that contains transcription unit sets TUS₁, TUS₂, TUS₃, and TUS₄. In other embodiments of the disclosure, the plant is transformed with a plasmid that contains only transcription unit set TUS₁.

In some embodiments of the disclosure, the plant is transformed with a plasmid that contains transcription unit sets TUS₁, and TUS₂. In other embodiments of the disclosure, the plant is transformed with a plasmid that contains transcription unit sets TUS₁, TUS₂, and TUS₃.

In other embodiments of the disclosure, the plant is transformed with a plasmid that contains transcription unit sets TUS₁, TUS₂, and TUS₄.

In other embodiments of the disclosure, the plant is transformed with a plasmid that contains transcription unit sets TUS₁, and TUS₃. In other embodiments of the disclosure, the plant is transformed with a plasmid that contains transcription unit sets TUS₁, TUS₃, and TUS₄.

In other embodiments of the disclosure, the plant is transformed with a plasmid that contains transcription unit sets TUS₁, and TUS₄. In some embodiments of the disclosure, transgenic plants are prepared by crossing a first plant that has been transformed with a plasmid containing one or more transcription unit sets with a second untransformed plant. In other embodiments of the disclosure, transgenic plants are prepared by crossing a first plant that has been transformed with a plasmid containing one or more but not all transcription unit sets required for micelle formation in vivo with a second plant having one or more transcription unit sets, wherein at least one of the transcription unit sets is present in the second plant and not present in the first plant.

In some embodiments of the disclosure, transgenic plants are prepared by crossing a first plant that has been transformed with a plasmid containing one or more transcription unit sets enabling micelle formation in vivo with a second plant having another trait, such as herbicide resistance or pest resistance.

In some embodiments of the disclosure, transgenic plants are prepared by growing progeny generations of a plant that has been transformed with a plasmid containing one or more transcription unit sets enabling micelle formation in vivo. In other embodiments, transgenic plants are prepared by growing progeny generations of a transgenic plant produced by crossing one or more plants that have been transformed with a plasmid containing one or more transcription unit sets enabling micelle formation in vivo.

Further to this example shown in FIG. 2 as an embodiment, the promoters in the four transcription unit sets include the promoters of genes coding for soybean Glycinin1, soybean β-conglycinin1, soybean Glycinin4, soybean Bowman-Birk protease inhibitor, Agrobacterium nopaline synthase, soybean Glycinin5, soybean lectin, and soybean Glycinin3. For clarity and brevity, the promoters of genes coding for soybean Glycinin1 is shown and annotated in FIG. 2 as GY1 PROMOTER. Also for clarity and brevity, the soybean β-conglycinin1 is shown and annotated in FIG. 2 as CG1 promoter. Further for clarity and brevity, the soybean Glycinin4 is shown and annotated in FIG. 2 as GY4 promoter. Yet further for clarity and brevity, the Bowman-Birk protease inhibitor promoter is shown and annotated in FIG. 2 as D-II promoter. Yet further for clarity and brevity, the Agrobacterium nopaline synthase is shown and annotated in FIG. 2 as NOS promoter. Yet further for clarity and brevity, the soybean Glycinin5 is shown and annotated in FIG. 2 as GY5 promoter. Yet further for clarity and brevity, the soybean lectin is shown and annotated in FIG. 2 as LE1 promoter. Yet further for clarity and brevity, the soybean Glycinin3 is shown and annotated in FIG. 2 as GY3 promoter.

In other embodiments and examples, promoters in one or more of the four transcription unit sets include a promoter capable of initiating transcription in plant cells whether or not an origin of the promoter is a plant cell. For example, Agrobacterium promoters are functional in plant cells. The promoters capable of initiating transcription in plant cells include promoters obtained from plants, plant viruses and bacteria such as Agrobacterium.

As specific examples of promoters under developmental control include promoters that preferentially initiate transcription in certain tissues, such as leaves, roots, or seeds. Such promoters are referred to as “tissue preferred”. Also as specific examples of promoters that initiate transcription only in certain tissues are referred to as “tissue specific”. Further as a specific example, a “cell type specific” promoter primarily drives expression in certain cell types in one or more organs, for example, vascular cells in roots or leaves. Yet further a specific example, an “inducible” or “repressible” promoter is a promoter which is under environmental control. Examples of environmental conditions that may affect transcription by inducible or repressible promoters include anaerobic conditions, or certain chemicals, or the presence of light. Tissue preferred, tissue specific, cell type specific, and inducible or repressible promoters constitute the class of “non-constitutive” promoters. A “constitutive” promoter is a promoter which is active under most conditions.

Returning to this example in FIG. 2 as an embodiment, the transcriptional terminators in the four transcription unit sets include the termination sequence of the nopaline synthase gene, shown and annotated in FIG. 2 as NOS terminator. In other embodiments, the transcriptional terminators in one or more of the four transcription unit sets includes transcriptional terminators from the native soybean Glycinin genes, or any other plant transcriptional terminators.

In this example as an embodiment, the T-DNA used to transform a plant also includes DNA encoding an origin of replication, a gene conferring antibiotic resistance, a right boundary for the T-DNA, and a left boundary for the T-DNA, shown and annotated in FIG. 2 as pUC origin, ampicillin resistance, RB, and LB, respectively. In this embodiment, the gene conferring antibiotic resistance is a gene conferring resistance to the antibiotic ampicillin. In other embodiments, the gene conferring antibiotic resistance is a gene conferring resistance to any other antibiotic, including kanamycin and chloramphenicol.

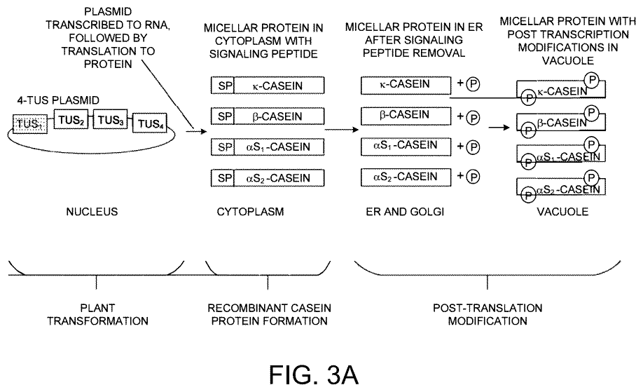

Referring now to FIG. 3A, therein is shown an example with additional details from the plant transformation to the post-translation modification. The plant transformation and the post-translation modification are also described in FIG. 1. The example depicted in FIG. 3A also depicts the recombinant casein protein formation, also described in FIG. 1. As a specific example, FIG. 3A schematically illustrates the transcription of casein proteins from genes in TUS₁ as well as post transcriptional alterations that occur as the proteins move towards their subcellular specific destination encoded by the common signal peptide.

In the example shown in FIG. 3A, the purpose of transcription unit set 1 is forming casein micelles in vivo in an embodiment. In this example, the plant transformation depicts a plant transformed using a T-DNA including four transcription unit sets shown and annotated in FIG. 3A as 4-TUS plasmid. The T-DNA includes transcription unit set 1, shown and abbreviated in FIG. 3A as TUS₁, which includes one transcription unit for each of the four casein proteins found in a casein micelle, with each transcription unit including DNA encoding the same plant-derived signal peptide, a promoter and a transcriptional terminator as described in FIG. 2. Upon transcription and translation of TUS₁ in the transgenic plant during the recombinant casein protein formation, the four recombinant casein proteins (αS₁-casein, αS₂-casein, β-casein, and κ-casein) are formed in the cytoplasm, each including a signal peptide that localizes the recombinant protein to a specific tissue, for example the secretory pathway and protein storage vacuoles, in the plant cell. In this example, the signal peptide is removed from the recombinant casein proteins during post-translational modification that occurs in the endoplasmic reticulum, abbreviated as ER, of the plant cell.

Continuing this example and embodiment for the post-translation modification, phosphorylation occurs on the recombinant casein proteins prior to, during, or after migration to a specific tissue. The phosphorylation is shown in FIG. 3A as circles enclosing the letter “P” that are added to and then attached to each of the recombinant casein proteins to form phosphorylated casein proteins. The phosphorylated casein proteins are then localized to the vacuole where micelle assembly occurs in vivo. In some embodiments, proteins encoded by TUS₂ transcription units (not shown) are also phosphorylated, glycosylated, or a combination thereof. In other embodiments, the casein proteins encoded by TUS₄ transcription units (not shown) are also phosphorylated or glycosylated or both. In other embodiments, no post-translational modifications occur to proteins encoded by TUS₁, TUS₂, TUS₃, or TUS₄ (not shown). As another example and embodiment, a kinase gene may optionally be included to generate a kinase protein that ensures phosphorylation of the casein proteins encoded by TUS₄ transcription units (not shown).

Referring now to FIG. 3B, therein is shown an example with additional details for the in vivo formation. The in vivo formation is also described in FIG. 1. As a specific example, FIG. 3B schematically illustrates the in vivo formation of recombinant micelles inside a plant cell.

Upon localization to the vacuole, each of the four recombinant casein proteins assemble with the other recombinant casein proteins to form micelles in vivo. In this example, the outer layer of the micelle is enriched in recombinant κ-casein shown and abbreviated in FIG. 3B as κ, and the inner matrix of the micelle includes recombinant αS₁-casein and αS₂-casein, shown and abbreviated as αS₁ and αS₂, respectively, in FIG. 3B, and β-casein, shown and abbreviated in FIG. 3B as 3.

Referring now to FIG. 3C, therein is shown an example of a schematic illustration of a transcription of proteins which impart herbicide resistance to the transformed plant. FIG. 3C depicts an example of the purpose of transcription unit set 2 in an embodiment.

In this example, a plant is transformed using a T-DNA including four transcription unit sets shown and annotated in FIG. 3C as 4-TUS plasmid. The T-DNA includes transcription unit set 2, shown in FIG. 3C and abbreviated as TUS₂ which includes one transcription unit that includes DNA encoding phosphinothricin acetyltrasnferase that imparts herbicide resistance and allow for selection of transformed cells producing micelles, shown and abbreviated as AC-PT in FIG. 3C, and a promoter and a transcriptional terminator (not shown).

Referring now to FIG. 3D, therein is shown an example of a schematic illustration of suppression of native seed storage proteins by interference RNA (RNAi) transcribed by a portion of the plasmid of FIG. 1. As a specific example, FIG. 3D schematically illustrates suppression of native seed storage proteins by RNAi transcribed by one or more genes in TUS₃.

FIG. 3D depicts an example of the purpose of transcription unit set 3 in an embodiment. In this example, a plant is transformed using a T-DNA including four transcription unit sets, shown and annotated in FIG. 3D as 4-TUS plasmid. The T-DNA includes transcription unit set 3, shown and abbreviated in FIG. 3D as TUS₃, which includes one or more transcription units that yield untranslated RNA molecules that suppress native seed protein gene translation thereby freeing cellular resources to produce micelles in vivo. Transcription of the DNA in TUS₃ yields RNAi, shown and abbreviated in FIG. 3D as RNAi, that targets messenger RNA of native plant proteins or native plant peptides, shown and annotated in FIG. 3D as mRNA NATIVE PROTEIN, and suppresses the expression of those messenger RNAs through messenger RNA degradation such that the recombinant casein proteins encoded by TUS₁, described in FIG. 1, FIG. 3A, and FIG. 3B, can be translated at higher quantities, thereby yielding higher concentrations of micelles in vivo (not shown). In some embodiments, DNA encoding RNAi is operably linked to a constitutive promoter or an inducible promoter (not shown), such as for example a nopaline synthase promoter or soybean α′ subunit of β-conglycinin, such that the suppression of native seed protein gene translation by RNAi can be regulated.

Referring now to FIG. 3E and FIG. 3F, therein are shown examples of schematic illustrations of a transcription of a portion of the plasmid of FIG. 1 and resulting proteins used to alter the intracellular conditions of the plant cell. As specific examples, FIG. 3E and FIG. 3F schematically illustrate the transcription of TUS₄ genes and the resulting proteins used to alter the conditions in the cytoplasm of the cell.

FIG. 3E depicts an example of the purpose of transcription unit set 4 in an embodiment. In this example, a plant is transformed using a T-DNA including four transcription unit sets, shown and annotated in FIG. 3E as 4-TUS plasmid. The T-DNA includes transcription unit set 4, shown and abbreviated in FIG. 3E as TUS₄, which includes one or more transcription units that encode proteins which increase the concentration of intracellular minerals, including calcium and phosphate. In this example, TUS₄ includes one transcription unit, a TU₄₋₁, that includes a promoter, DNA encoding oxalate decarboxylase, and a transcriptional terminator (not shown). Transcription and translation of TU₄₋₁ yields the enzyme oxalate decarboxylase, shown and abbreviated in FIG. 3E as OD, that breaks down the calcium oxalate and increases calcium levels in the plant cell. The increased intracellular calcium enhances the formation of recombinant casein micelles in the plant cell (not shown).

FIG. 3F depicts an example of the purpose of transcription unit set 4 in an embodiment. In this example, a plant is transformed using a T-DNA including four transcription unit sets, shown and annotated in FIG. 3F as 4-TUS plasmid. The T-DNA includes transcription unit set 4, shown and abbreviated in FIG. 3F as TUS₄, which includes one or more transcription units that encode proteins which increase the concentration of intracellular minerals, including calcium and phosphate. In this example, TUS₄ includes one transcription unit, a TU₄₋₂, that includes a promoter, DNA encoding a phytase enzyme, and a transcriptional terminator (not shown). Transcription and translation of TU₄₋₂ yields the phytase enzyme, shown and abbreviated in FIG. 3F as PE, that breaks down the phytic acid and increases phosphate levels in the plant cell. The increased intracellular phosphate enhances the formation of recombinant casein micelles in the plant cell (not shown).

Referring now to FIG. 3G, therein is shown an example of further additional details of the in vivo formation. The in vivo formation is also described in FIG. 1, FIG. 3A, and FIG. 3B. As a specific example, FIG. 3G schematically illustrates the in vivo formation of recombinant micelles inside a plant cell.

In the example shown in FIG. 3G, the in vivo formation of recombinant micelles inside a plant cell in which the four micellar proteins are produced by transcription and translation of transcription unit set 1 as depicted and described in FIG. 3A. The levels of calcium in plant cell vacuoles is increased by the presence of oxalate decarboxylase produced by transcription and translation of transcription unit set 4 as depicted and described in FIG. 3E. In this example, the four casein proteins encoded by transcription unit 1 are phosphorylated and localized to the plant cell vacuole where the intracellular calcium and the intracellular phosphate enhances the formation of recombinant casein micelles in the plant cell vacuole.

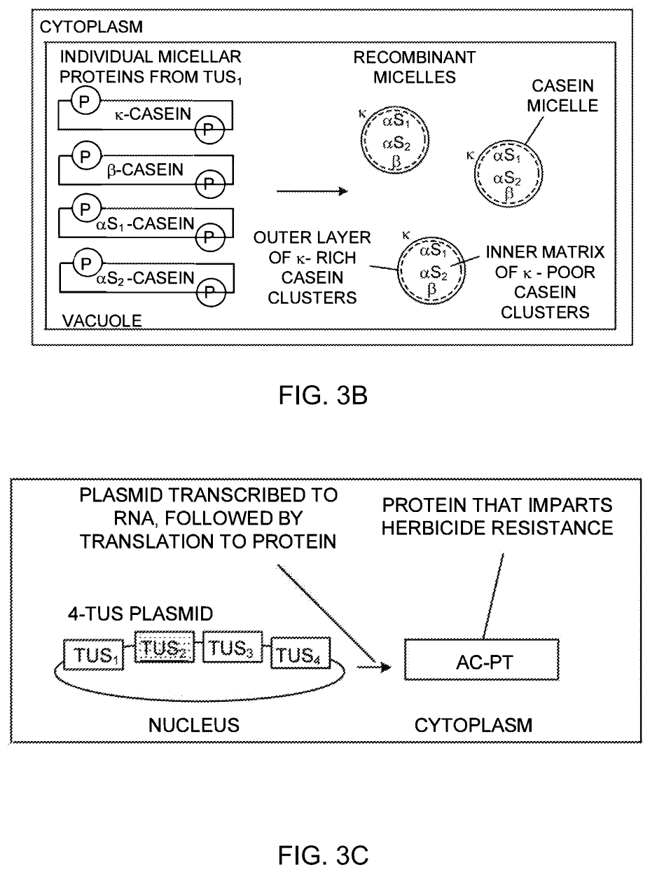

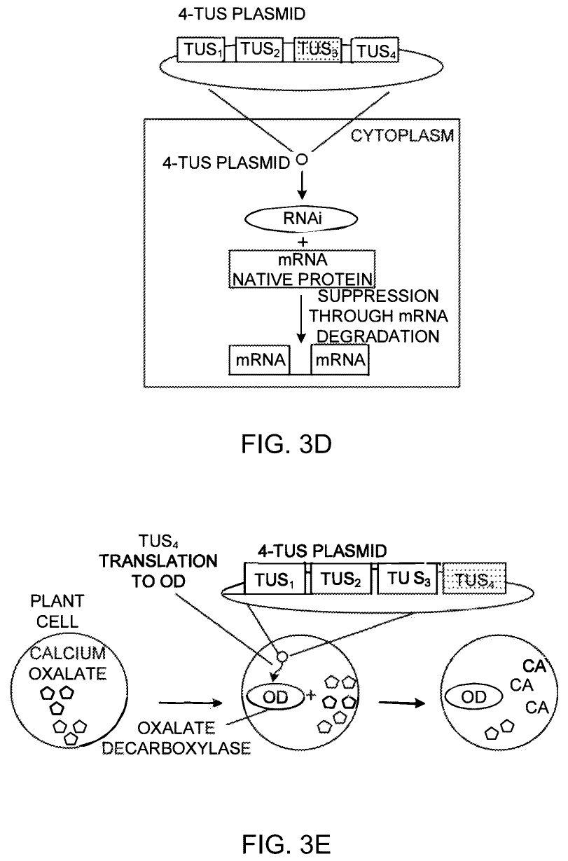

Aspects of the disclosure can be further illustrated by a specific embodiment in which a casein micelle is assembled in vivo from its constituent proteins in Arabidopsis thaliana as further described in FIG. 4 and FIG. 5.

Referring now to FIG. 4, therein is shown an example of a schematic illustration of a portion of a plasmid in Arabidopsis. The example shown in FIG. 4 is also described in FIG. 2. As a specific example, FIG. 4 schematically illustrates elements of plasmids that encode micellar component proteins. Transcription units depicted are components of TUS₁ in Arabidopsis.

The example in FIG. 4 depicts a transcription unit set which can be used for creation of casein micelles in vivo in Arabidopsis thaliana. The transcription unit set includes one transcription unit for each of the four casein proteins found in a casein micelle, abbreviated and shown in FIG. 4 as TU₁₋₁, TU₁₋₂, TU₁₋₃, and TU₁₋₄. The transcription unit set abbreviated and shown in FIG. 4 as TUS₁. Each of the four transcription units includes a promoter, a plant-derived N-terminal signal peptide, DNA encoding one of the four proteins found in a casein micelle, and a transcriptional terminator.

Continuing this example, TU_{1.1} includes a double 35S promoter containing the tobacco mosaic virus omega leader sequence (SEQ ID NO:29), a signal peptide from the Arabidopsis CLV3 gene (SEQ ID NO:27), the αS₁-casein coding sequence codon optimized for expression in Arabidopsis with a C-terminal HDEL peptide for retention in the endoplasmic reticulum (SEQ ID NO:5), and the nopaline synthase terminator (SEQ ID NO:35), annotated and shown in FIG. 4 as 2X35S promoter+TMVΩ, signal peptide, αS₁ casein, and NOS terminator, respectively.

Further continuing this example, TU₁₋₂ includes a 35S short promoter containing a truncated version of the cauliflower mosaic virus promoter and the tobacco mosaic virus omega leader sequence (SEQ ID NO:31), a signal peptide (SEQ ID NO:27), the β-casein coding sequence codon optimized for expression in Arabidopsis with a C-terminal HDEL peptide for retention in the endoplasmic reticulum (SEQ ID NO:7), and the nopaline synthase terminator (SEQ ID NO:35), abbreviated and shown in FIG. 4 as 35S SHORT PROMOTER+TMVΩ, SIGNAL PEPTIDE, β-CASEIN, and NOS TERMINATOR, respectively.

Further continuing this example, TU₁₋₃ includes the mannopine synthase promoter from Agrobacterium tumefaciens (SEQ ID NO:32), a signal peptide (SEQ ID NO:27), the κ-casein coding sequence codon optimized for expression in Arabidopsis with a C-terminal HDEL peptide for retention in the endoplasmic reticulum (SEQ ID NO:6), and the nopaline synthase terminator (SEQ ID NO:35), abbreviated and shown in FIG. 4 as MANNOPINE SYNTHASE PROMOTER (A. Tumefaciens), SIGNAL PEPTIDE, x-CASEIN, and NOS TERMINATOR, respectively.

Further continuing this example, TU₁₋₄ includes the mannopine synthase promoter from Agrobacterium tumefaciens, a signal peptide (SEQ ID NO:32), the αS₂-casein coding sequence codon optimized for expression in Arabidopsis with a C-terminal HDEL peptide for retention in the endoplasmic reticulum (SEQ ID NO:8), and the nopaline synthase terminator (SEQ ID NO:35), abbreviated and shown in FIG. 4 as MANNOPINE SYNTHASE PROMOTER (A. tumefaciens), SIGNAL PEPTIDE, αS₂ CASEIN, and NOS TERMINATOR, respectively.

Referring now to FIG. 5, therein is shown an example of a schematic illustration of a portion of a plasmid in Arabidopsis for a screenable marker in plants. As a specific example, FIG. 5 schematically illustrates elements of plasmids that provide for a screenable marker in plants. Transcription units depicted are components of TUS₂ in Arabidopsis.

The example shown in FIG. 5 depicts a transcription unit set which can be used to identify plant cells that have been transformed. The transcription unit set, abbreviated and shown in FIG. 5 as TUS₂, includes a single transcription unit, abbreviated and shown in FIG. 5 as TU₂₋₁.

Continuing this example for a portion of the plant transformation shown in FIG. 5 as an embodiment, TU₂₋₁ includes the nopaline synthase constitutive promoter (SEQ ID NO:28), the enhanced green fluorescence protein coding sequence modified to enhance fluorescence brightness and codon optimized for expression in Arabidopsis (SEQ ID NO:33), and the nopaline synthase terminator (SEQ ID NO:35), abbreviated and shown in FIG. 5 as NOS PROMOTER, EGFP, and NOS TERMINATOR, respectively.

As a specific example, subsequent steps in the plant transformation for creation of casein micelles in vivo in Arabidopsis thaliana, a plasmid including TUS₁ and TUS₂ can be introduced into Arabidopsis thaliana cotyledons using Agrobacterium tumefaciens and the FAST transient expression method. Seedlings are soaked in a solution containing Agrobacterium two days after germination which results in some cotyledon cells being transformed. Transformed Arabidopsis cells can be identified as containing the T-DNA by observing fluorescence exhibited by the enhanced green fluorescence protein. Successfully transformed Arabidopsis cells display green fluorescence while unsuccessfully transformed cells show little or no green fluorescence.

Also as a specific example of the in vivo formation of micelles in Arabidopsis thaliana as an embodiment, immunogold labeling techniques can be used to identify the location and morphology of the casein micelles formed in vivo. For this example for the in vivo formation of micelles as an embodiment, embryonic tissue can be obtained from Arabidopsis thaliana that has been transformed with a plasmid including TUS₁, and optionally TUS₂, shown in FIG. 4 and FIG. 5, respectively. The embryonic tissue can be treated with casein-specific antibodies using immunogold labeling techniques, and imaged with transmission electron microscopy to identify the location and morphology of the micelles formed in vivo. In tissue obtained from the transformed Arabidopsis thaliana, the casein micelles are visualized as gold-antibody labeled subcellular structures that range in size from 50 nm to 600 nm, which is similar to the size of bovine casein micelles. As a control, no casein micelles are visualized using immunogold labeling techniques in tissue obtained from untransformed Arabidopsis thaliana.

Continuing this specific example of the in vivo formation of micelles in Arabidopsis thaliana as an embodiment, protein extraction and high performance liquid chromatography (HPLC) analysis can be used to evaluate the protein composition of the casein micelles formed in vivo. In this example for the in vivo formation of micelles as an embodiment, embryonic tissue can be obtained from Arabidopsis thaliana that has been transformed with a plasmid including TUS₁, and optionally TUS₂, shown in FIG. 4 and FIG. 5, respectively. Proteins extracted from the embryonic tissue can be separated using HPLC and detected by ultraviolet absorbance. Proteins extracted from the transformed Arabidopsis thaliana tissue and subjected to HPLC analysis show peaks associated with each four proteins found in a casein micelle, including αS₁ casein, αS₂ casein, 3 casein, and κ casein, that display retention times similar to those reported by Bordin et al. for each of the four casein proteins found in bovine casein micelles. As a control, proteins extracted from the untransformed Arabidopsis thaliana tissue and subjected to HPLC analysis do not show peaks associated with any of the four casein proteins.

Further continuing this specific example of the in vivo formation of micelles in Arabidopsis thaliana as an embodiment, the amount of each casein protein found in micelles formed in vivo can be quantified by measuring the area under the peaks produced upon HPLC analysis. Quantification of the peaks produced upon HPLC analysis of proteins extracted from transformed Arabidopsis thaliana produces measurements showing that αS₁ casein is the most abundant, followed by p casein as the next most abundant, then αS₂ casein and κ casein as the least abundant casein proteins, which correlates to the relative abundances of each of the four casein proteins in bovine casein micelles as previously reported in the Handbook of Dairy Foods and Nutrition, Table 1.1.

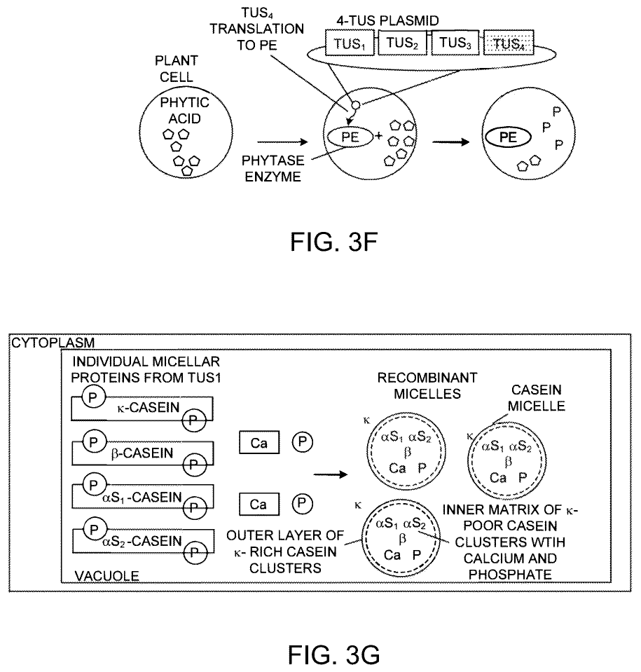

Aspects of the disclosure can be further illustrated by a specific embodiment in which a casein micelle is assembled in vivo from its constituent proteins in soybean and further described in FIG. 6 through FIG. 9.

Referring now to FIG. 6, therein is shown an example of a schematic illustration of a portion of a plasmid in soybean. As a specific example, FIG. 6 schematically illustrates elements of plasmids that encode micellar component proteins. Transcription units depicted are components of TUS₁ in soybean.

In this example, FIG. 6 depicts a transcription unit set which can be used for creation of casein micelles in vivo in soybean. The transcription unit set includes one transcription unit for each of the four casein proteins found in a casein micelle, abbreviated and shown in FIG. 4 as TU₁₋₁, TU₁₋₂, TU₁₋₃, and TU₁₋₄. The first transcription unit set is abbreviated and shown in FIG. 6 as TUS₁. Each of the four transcription units includes a promoter, a plant-derived N-terminal signal peptide, DNA encoding one of the four proteins found in a casein micelle, and a transcriptional terminator.

Continuing this example for a portion of the plant transformation shown in FIG. 6 as an embodiment, TU_{1.1} includes a promoter from the soybean glycinin GY1 gene (SEQ ID NO:13), a signal peptide (SEQ ID NO:26), the αS₁ casein coding sequence codon optimized for expression in soybean (SEQ ID NO:1), and the nopaline synthase terminator (SEQ ID NO:35), abbreviated and shown in FIG. 6 as GY1 PROMOTER, SIGNAL PEPTIDE, αS₁ CASEIN, and NOS TERMINATOR, respectively.

Further continuing this example for a portion of the plant transformation shown in FIG. 6 as an embodiment, TU₁₋₂ includes the promoter from the soybean CG1 gene (SEQ ID NO:14), a signal peptide (SEQ ID NO:26), the β casein coding sequence codon optimized for expression in soybean (SEQ ID NO:3), and the nopaline synthase terminator (SEQ ID NO:35), abbreviated and shown in FIG. 6 as CG1 PROMOTER, SIGNAL PEPTIDE, β CASEIN, and NOS TERMINATOR, respectively.

Further continuing this example for a portion of the plant transformation shown in FIG. 6 as an embodiment, TU₁₋₃ includes the promoter from the soybean glycinin GY4 gene (SEQ ID NO:15), a signal peptide (SEQ ID NO:26), the κ casein coding sequence codon optimized for expression in soybean (SEQ ID NO:2), and the nopaline synthase terminator (SEQ ID NO:35), abbreviated and shown in FIG. 6 as GY4 PROMOTER, SIGNAL PEPTIDE, K CASEIN, and NOS TERMINATOR, respectively.

Further continuing this example for a portion of the plant transformation shown in FIG. 6 as an embodiment, TU₁₋₄ includes the promoter from the soybean D-II Bowman-Birk proteinase isoinhibitor gene (SEQ ID NO:16), a signal peptide (SEQ ID NO:26), the αS₂ casein coding sequence codon optimized for expression in soybean (SEQ ID NO:4), and the nopaline synthase terminator (SEQ ID NO:35), abbreviated and shown in FIG. 6 as D-II PROMOTER, SIGNAL PEPTIDE, αS₂ CASEIN, and NOS TERMINATOR, respectively.

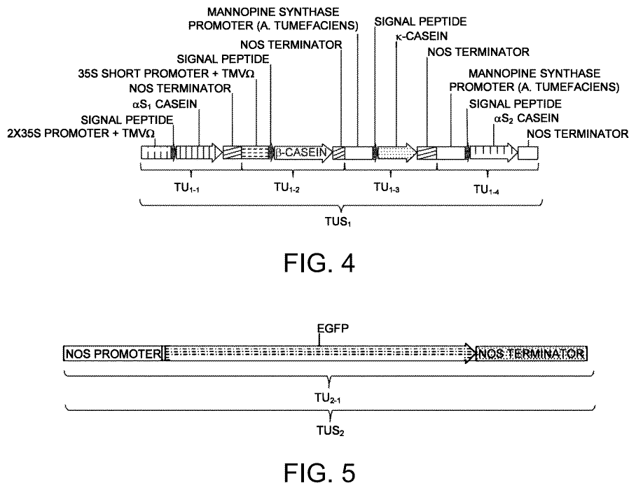

Referring now to FIG. 7, therein is shown an example of a schematic illustration of a portion of a plasmid in soybean for herbicide resistance in plants. As a specific example, FIG. 7 schematically illustrates elements of plasmids that provide for herbicide resistance in plants. Transcription units depicted are components of TUS₂ in soybean.

FIG. 7 is an example of a portion of the plant transformation that depicts a transcription unit set which can be used to select for plant cells that have been transformed. The transcription unit set abbreviated and shown in FIG. 7 as TUS₂ includes a single transcription unit abbreviated and shown in FIG. 7 as TU₂₋₁.

Continuing this example for a portion of the plant transformation shown in FIG. 7 as an embodiment, TU₂₋₁ includes nopaline synthase promoter (SEQ ID NO:28), the phosphinothricin acetyltransferase coding sequence codon optimized for expression in soybean (SEQ ID NO:34) which confers resistance to the herbicide glufosinate, and the nopaline synthase terminator (SEQ ID NO:35), abbreviated and shown in FIG. 7 as NOS PROMOTER, PHOSPHINOTHRICIN ACETYLTRANSFERASE, and NOS TERMINATOR, respectively.

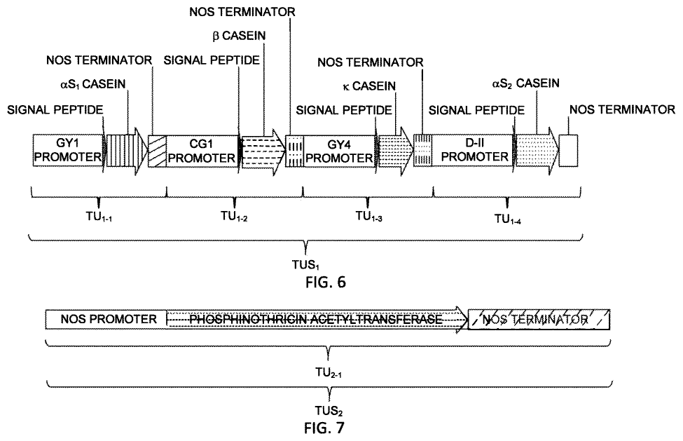

Referring now to FIG. 8, therein is shown an example of a schematic illustration of a portion of a plasmid in soybean for suppression of native seed storage proteins in plants. As a specific example, FIG. 8 schematically illustrates elements of plasmids that provide for suppression of native seed storage proteins in plants. Transcription units depicted are components of TUS₃ in soybean.

FIG. 8 is an example of a portion of the plant transformation that depicts a transcription unit set which can be used for enhancing the creation of casein micelles in vivo in soybean. The third transcription unit set abbreviated and shown in FIG. 8 as TUS₃ includes two transcription units abbreviated and shown in FIG. 8 as TU₃₋₁ and TU₃₋₂. The transcription of TU₃₋₁ and TU₃₋₂ produces RNA with a hairpin structure where the arms are homologous to a portion of a native soybean gene or gene family and are sufficient to cause down regulation of those native genes or gene families (not shown).

Continuing this example for a portion of the plant transformation shown in FIG. 8 as an embodiment, TU₃₋₁ includes a promoter from the soybean glycinin GY4 gene (SEQ ID NO:15), a portion of the soybean glycinin GY1 coding sequence that is lacking a start codon and is highly homologous among the glycinin gene family (SEQ ID NO:24), the potato IV2 intron (SEQ ID NO:25), the antisense of the soybean glycinin GY1 sequence (SEQ ID NO:17), and the nopaline synthase terminator (SEQ ID NO:35), abbreviated and shown in FIG. 8 as GY4 PROMOTER, GY1 SENSE, IV2 INTRON, GY1 ANTISENSE, and NOS TERMINATOR, respectively.

Further continuing this example for a portion of the plant transformation shown in FIG. 8 as an embodiment, TU₃₋₂ includes a promoter from the soybean glycinin GY5 gene (SEQ ID NO:18), a portion of the soybean β-conglycinin 1 coding sequence that is lacking a start codon and is highly homologous among the β-conglycinin gene family (SEQ ID NO:19), the potato IV2 intron (SEQ ID NO:25), the antisense of the soybean β-conglycinin 1 sequence (SEQ ID NO:20), and the nopaline synthase terminator (SEQ ID NO:35), abbreviated and shown in FIG. 8 as GY5 PROMOTER, CG1 SENSE, IV2 INTRON, CG1 ANTISENSE, and NOS TERMINATOR, respectively.

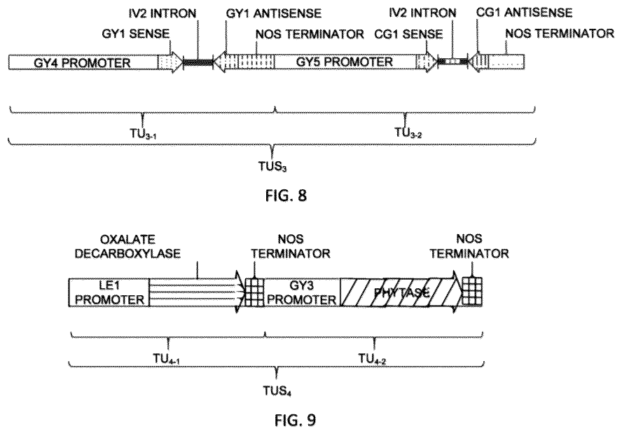

Referring now to FIG. 9, therein is shown an example of a schematic illustration of a portion of a plasmid in soybean for regulating cytoplasmic concentrations of minerals which can enhance micelle formation. As a specific example, FIG. 9 schematically illustrates elements of plasmids that regulate cytoplasmic concentrations of minerals which can enhance micelle formation. Transcription units depicted are components of TUS₄ in soybean.

FIG. 9 is an example of a portion of the plant transformation that depicts a transcription unit set which can be used for enhancing the creation of casein micelles in vivo in soybean. The fourth transcription unit set abbreviated and shown in FIG. 9 as TUS₄ includes two transcription units abbreviated and shown in FIG. 9 as TU₄₋₁ and TU₄₋₂. Proteins encoded by TU₄₋₁ and TU₄₋₂ alter the intracellular environment in a manner that optimizes the formation of micelles in vivo.

Continuing this example for a portion of the plant transformation shown in FIG. 9 as an embodiment, TU₄₋₁ includes a promoter from the soybean LE1 gene (SEQ ID NO:23), a coding sequence for oxalate decarboxylase from Flammulina velutipes codon optimized for expression in soybean (SEQ ID NO:12), and nopaline synthase terminator (SEQ ID NO:35), abbreviated and shown in FIG. 9 as LE1 PROMOTER, OXALATE DECARBOXYLASE CDS, and NOS TERMINATOR, respectively.

Further continuing this example for a portion of the plant transformation shown in FIG. 9 as an embodiment, TU₄₋₂ includes the glycinin GY3 promoter (SEQ ID NO:30), the coding sequence for a soybean phytase enzyme (SEQ ID NO:11), and the nopaline synthase terminator (SEQ ID NO:35), abbreviated and shown in FIG. 9 as GY3 PROMOTER, PHYTASE, and NOS TERMINATOR, respectively.

In this example, subsequent steps in the plant transformation for creation of casein micelles in vivo in soybean, a plasmid including TUS₁, TUS₂, and optionally TUS₃, and optionally TUS₄, shown in FIG. 6, FIG. 7, FIG. 8, and FIG. 9, respectively, can be introduced into soybean callus using standard biolistic transformation methods. Transformed soybean plants can be selected on a medium containing glufosinate herbicide, and the genomes of transformed soybean plants can be screened for insertion of the plasmid using standard PCR mapping methods. Transformed soybean plants including TUS₁, TUS₂, and optionally TUS₃, and optionally TUS₄ in their genome can be transferred to a greenhouse for seed production.

In the example of the in vivo formation of micelles in soybean as an embodiment, immunogold labeling techniques can be used to identify the location and morphology of the casein micelles formed in vivo. As it relates to this example for the in vivo formation of micelles as an embodiment, tissue can be obtained from soybean plants that have been transformed with a plasmid including TUS₁, TUS₂, and optionally TUS₃, and optionally TUS₄, shown in FIG. 6, FIG. 7, FIG. 8, and FIG. 9, respectively. The tissue can be treated with casein-specific antibodies using standard immunogold labeling techniques, and imaged with transmission electron microscopy to identify the location and morphology of the micelles formed in vivo. In tissue obtained from the transformed soybean plants, the casein micelles are visualized as gold-antibody labeled subcellular structures that range in size from 50 nm to 600 nm, which is similar to the size of bovine casein micelles. As a control, no casein micelles are visualized using immunogold labeling techniques in tissue obtained from untransformed soybean plants.

Continuing this example of the in vivo formation of micelles in soybean as an embodiment, protein extraction and polyacrylamide gel electrophoresis analysis can be used to evaluate the protein composition of the casein micelles formed in vivo. For this example for the in vivo formation of micelles as an embodiment, tissue can be obtained from soybean plants that have been transformed with a plasmid including TUS₁, TUS₂, and optionally TUS₃, and optionally TUS₄, shown in FIG. 6, FIG. 7, FIG. 8, and FIG. 9, respectively. Proteins extracted from the transformed soybean plant tissue and subjected to polyacrylamide gel electrophoresis analysis show bands on the polyacrylamide gel corresponding in size to each of the four casein proteins found in a casein micelle, including αS₁ casein, αS₂ casein, β casein, and κ casein. As a control, proteins extracted from untransformed soybean plant tissue and subjected to polyacrylamide gel electrophoresis analysis do not show bands on the polyacrylamide gel corresponding to the four casein proteins.

Further continuing this example of the in vivo formation of micelles in soybean as an embodiment, protein extraction and HPLC analysis can be used to evaluate the protein composition of the casein micelles formed in vivo. For this example for the in vivo formation of micelles as an embodiment, tissue can be obtained from soybean plants that have been transformed with a plasmid including TUS₁, TUS₂, and optionally TUS₃, and optionally TUS₄, shown in FIG. 6, FIG. 7, FIG. 8, and FIG. 9, respectively. Proteins extracted from the transformed soybean plant tissue can be separated using HPLC and detected by ultraviolet absorbance. Proteins extracted from the transformed soybean plant tissue and subjected to HPLC analysis show peaks associated with each four proteins found in a casein micelle, including αS₁ casein, αS₂ casein, p casein, and κ casein, that display retention times similar to those reported by Bordin et al. for each of the four casein proteins found in bovine casein micelles. As a control, proteins extracted from the untransformed soybean plant tissue and subjected to HPLC analysis do not show peaks associated with the four casein proteins.

Further continuing this example of the in vivo formation of micelles in soybean as an embodiment, the amount of each casein protein found in micelles formed in vivo can be quantified by measuring the area under the peaks produced upon HPLC analysis. Quantification of the peaks produced upon HPLC analysis of proteins extracted from transformed soybean plant tissue produces measurements showing that αS₁ casein is the most abundant, followed by β casein as the next most abundant, then αS₂ casein and κ casein as the least abundant casein proteins, which correlates to the relative abundances of each of the four casein proteins in bovine casein micelles as previously reported in the Handbook of Dairy Foods and Nutrition, Table 1.1.

Further continuing this example of the in vivo formation of micelles in soybean as an embodiment, RNA analysis can be used to evaluate the suppression of native soybean seed genes during the formation of casein micelles in vivo. For this example for the in vivo formation of micelles as an embodiment, soybean plants that have been transformed with a plasmid including TUS₁, TUS₂, TUS₃, and optionally TUS₄, shown in FIG. 6, FIG. 7, FIG. 8, and FIG. 9, respectively, can be grown to the flowering stage in a greenhouse and soybean embryos removed from the flowering seed pods at 35 days using standard dissection techniques. The expression levels of native soybean seed genes can be analyzed using standard techniques for RNA extraction and sequencing. RNA analysis of the embryos from transformed soybean plants show at least a 10% reduction in the expression of one or more of the native soybean seed genes, including genes in the glycinin family (Glyma.03g163500, Glyma.19g164900, Glyma.10g037100, Glyma.13g123500, Glyma.19g164800) and genes in the β-conglycinin family (Glyma.10g246300, Glyma.20g148400, Glyma.20g148300, Glyma.20g146200, Glyma.20g148200, Glyma.10g246500, Glyma.10g028300, Glyma.02g145700). As a control, RNA analysis of embryos from untransformed soybean plants do not show a reduction in the expression of native soybean seed genes.

Further continuing this example of the in vivo formation of micelles in soybean as an embodiment, commercially available assays and X-ray fluorescence techniques can be used to evaluate calcium oxalate levels during the formation of casein micelles in vivo. As it relates to this example for the in vivo formation of micelles as an embodiment, soybean plants that have been transformed with a plasmid including TUS₁, TUS₂, and optionally TUS₃, and TUS₄, shown in FIG. 6, FIG. 7, FIG. 8, and FIG. 9, respectively, can be grown to the flowering stage in a greenhouse and soybean embryos removed from the flowering seed pods at 27 days using standard dissection techniques. The oxalate concentration can be measured using commercially available assays, and the calcium concentration can be measured using X-ray fluorescence. Embryos from transformed soybean plants show at least a 5% reduction in oxalate concentration and at least a 4% increase in calcium concentration as compared to control embryos from untransformed soybean plants, indicating that embryos from transformed soybean plants have at least 4% more available calcium compared to embryos from untransformed soybean plants.

Further continuing this example of the in vivo formation of micelles in soybean as an embodiment, commercially available assays can be used to evaluate phosphate levels during the formation of casein micelles in vivo. As it relates to this example for the in vivo formation of micelles as an embodiment, soybean plants that have been transformed with a plasmid including TUS₁, TUS₂, and optionally TUS₃, and TUS₄, shown in FIG. 6, FIG. 7, FIG. 8, and FIG. 9., respectively, can be grown to the flowering stage in a greenhouse and soybean embryos removed from the flowering seed pods at 27 days using standard dissection techniques. Embryos can be ground with a mortar and pestle, sonicated and centrifuged to produce a supernatant that can be tested for phosphatase levels using commercially available assays. Embryos from transformed soybean plants show at least a 5% increase in phosphatase levels as compared to control embryos from untransformed soybean plants, indicating that embryos from transformed soybean plants have at least 5% more available phosphate compared to embryos from untransformed soybean plants.

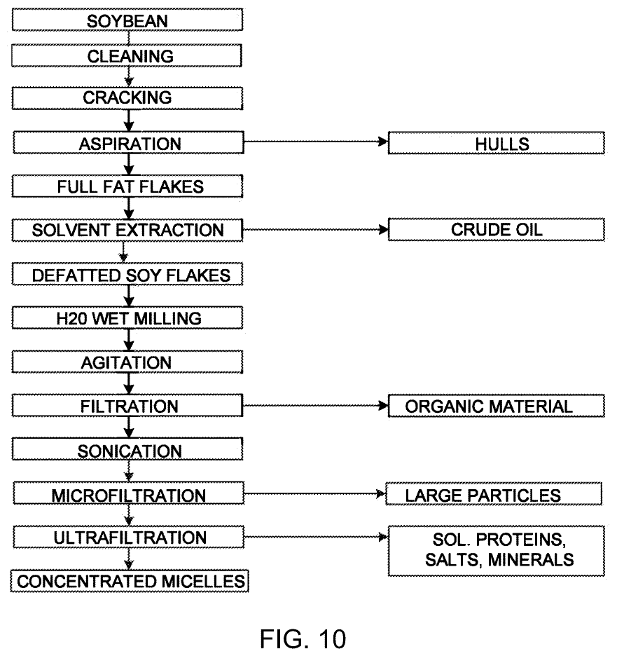

Aspects of the disclosure can be further illustrated by a specific embodiment in which micelles produced in vivo are purified as further described in FIG. 10.

Referring now to FIG. 10, therein is shown an example of a flow for the purification of micelles formed in vivo in soybean. Also, the flow in FIG. 10 is an example of isolating a recombinant micelle. Further in this example, FIG. 10 depicts a process where casein micelles produced in soybeans are purified from the plant tissue in a way that the micelles are still functional after the purification. The input material for the purification process is dried soybeans harvested from plants that have been transformed with a plasmid containing all four transcription unit sets, TUS₁, TUS₂, TUS₃, and TUS₄, described in FIG. 6, FIG. 7, FIG. 8, and FIG. 9, respectively. The input material for the purification process is shown in FIG. 10 and depicted as a rectangle enclosing the word “SOYBEAN”.

Continuing this example of the purification of micelles formed in vivo in soybean as an embodiment, the hulls are removed from the dried soybeans in a series of steps including cleaning, cracking, and aspiration, shown in FIG. 10 and depicted as rectangles enclosing the words “CLEANING”, “CRACKING” and “ASPIRATION”. In this embodiment, the hulls do not contain useful amounts of casein micelles and are discarded, shown in FIG. 10 and depicted as an arrow pointing to a rectangle enclosing the word “HULLS”.

Further continuing this example of the purification of micelles formed in vivo in soybean as an embodiment, the remaining material is flaked to increase the surface area and allow for faster aqueous or solvent infiltrations. The resulting flaked material is shown in FIG. 10 and depicted as a rectangle enclosing the words “FULL FAT FLAKES”.

Further continuing this example of the purification of micelles formed in vivo in soybean as an embodiment, the flaked material is then defatted with hexane using standard defatting equipment and solvent extraction techniques, shown in FIG. 10 and depicted as a rectangle enclosing the words “SOLVENT EXTRACTION”. Defatting can occur using any standard hexane based solvent, followed by desolventizing using flash or vapor-based processes. The resulting oil is removed, shown in FIG. 10 and depicted as an arrow pointing to a rectangle enclosing the words “CRUDE OIL”, leaving behind the defatted flakes, shown in FIG. 10 and depicted as an arrow pointing to a rectangle enclosing the words “DEFATTED SOY FLAKES”.

Further continuing this example of the purification of micelles formed in vivo in soybean as an embodiment, the defatted flakes are then mixed with water and wet milled, shown in FIG. 10 and depicted as an arrow pointing to a rectangle enclosing the words “H2O WET MILLING”. The milling process pulverizes the defatted flakes which releases the casein micelles and allows the micelles to come into contact with an aqueous medium. In addition to the milling process, the defatted flakes are also vigorously agitated to assist in the release of casein micelles into the water, shown in FIG. 10 and depicted as an arrow pointing to a rectangle enclosing the word “AGITATION”. The milling process and vigorous agitation of the defatted flakes yields a slurry where soybean material has been finely ground and many of the casein micelles have been released into suspension in the water (not shown). Additionally, many other proteins and carbohydrates are also dissolved in the water (not shown). In some embodiments, wet milling is done using perforated disc or colloid continuous flow mills.

Further continuing this example of the purification of micelles formed in vivo in soybean as an embodiment, the slurry is fed through a series of mesh screens to remove larger particles from the casein micelles, shown in FIG. 10 and depicted as an arrow pointing to a rectangle enclosing the word “FILTRATION”. In this embodiment, the slurry is first passed through a screen with 5 mm sieve openings (not shown), and then is passed through a screen with 0.5 mm sieve openings (not shown). The material trapped by the screens is discarded, shown in FIG. 10 and depicted as an arrow pointing to a rectangle enclosing the words “ORGANIC MATERIAL”.

Further continuing this example of the purification of micelles formed in vivo in soybean as an embodiment, the remaining material in the slurry that passed through both screens is then sonicated to break up aggregates of casein micelles such that the majority of micelles are not contacting other micelles, shown in FIG. 10 and depicted as an arrow pointing to a rectangle enclosing the word “SONICATION”. In some embodiments, continuous flow sonication with multiple sonicators in parallel are used to maximize flow rates.

Further continuing this example of the purification of micelles formed in vivo in soybean as an embodiment, after sonication the slurry is passed through a 2 μm microfiltration unit to eliminate larger particles while allowing casein micelles to pass through, shown in FIG. and depicted as an arrow pointing to a rectangle enclosing the word “MICROFILTRATION”. The material trapped by the microfiltration unit is discarded, shown in FIG. 10 and depicted as an arrow pointing to a rectangle enclosing the words “LARGE PARTICLES”. The remaining material that passed through the microfiltration unit is largely composed of casein micelles as well as dissolved proteins, salts and carbohydrates (not shown).

Further continuing this example of the purification of micelles formed in vivo in soybean as an embodiment, the material that passed through the microfiltration unit is then processed with an ultrafiltration unit that allows dissolved molecules lower than 100 nm in diameter to pass through while retaining casein micelles, shown in FIG. 10 and depicted as an arrow pointing to a rectangle enclosing the words “ULTRAFILTRATION”. In some embodiments, continuous flow ultrafiltration with multiple filters in parallel are used to maximize flow rates. The soluble proteins, salts and minerals that passed through the ultrafiltration unit are discarded, shown in FIG. 10 and depicted as an arrow pointing to a rectangle enclosing the words “SOL. PROTEINS, SALTS, MINERALS”.

Further continuing this example of the purification of micelles formed in vivo in soybean as an embodiment, the final output from this process is an aqueous liquid where the most common component after water is casein micelles, shown in FIG. 10 and depicted as an arrow pointing to a rectangle enclosing the words “CONCENTRATED MICELLES”. These micelles (not shown) retain their shape and function such that they can be used in downstream processes such as in making synthetic milk or cheese.

As additional examples for FIG. 10, a method of isolating recombinant micelles from a seed of a plant produced can include cleaning, and deshelling or dehulling seeds, flaking cleaned seeds to 0.005-0.02 inch thickness, solvent extraction of oil from the flake, desolventizing the flake without cooking and collecting the defatted, clean flake, separating micelles into an aqueous slurry by hydrating, agitating and wet milling the flake, passing the slurry through a series of mesh screens to remove particulate above 0.5 mm in size and collecting the permeate, sonication of the permeate from previous step, microfiltration of the product from previous step to remove particulate above 2 um in size, ultrafiltration of the permeate from previous step using a device that allows particles >100 nm in diameter to pass through in the ultrafiltration permeate, collecting the retentate of previous step which contains concentrated recombinant micelles.

Continuing with this example, the method of isolating recombinant micelles from a seed further includes centrifuging the retentate of a previous step to separate the micelles from the remainder of the retentate. Also the method continues from the ultrafiltration step to passing the slurry through an ultrafiltration device and collecting a permeate containing protein and other molecules and a retentate containing micelles and thereafter adding a diafiltration fluid to the retentate at substantially the same rate that the permeate is collected and passing said retentate through the ultrafiltration device. Yet further the method continues where the seed is milled from at least one plant selected from the group of plants consisting of maize, rice, sorghum, cowpeas, soybeans, cassava, coyam, sesame, peanuts, peas, cotton and yams.

The resulting method, process, apparatus, device, product, and system is cost-effective, highly versatile, and accurate, and can be implemented by adapting components for ready, efficient, and economical manufacturing, application, production, and utilization. Another important aspect of an embodiment of the present disclosure is that it valuably supports and services the historical trend of reducing costs, simplifying systems, and increasing yield.

### Construction of Expression Plasmids for Plant Transformation

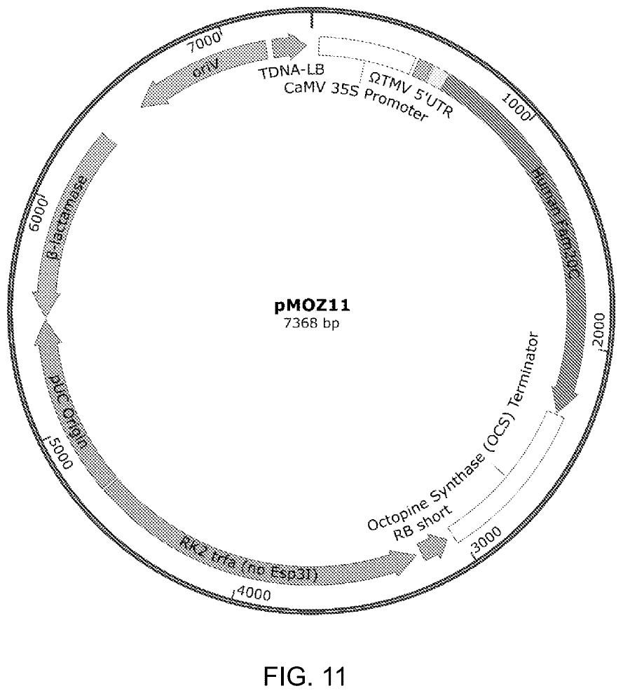

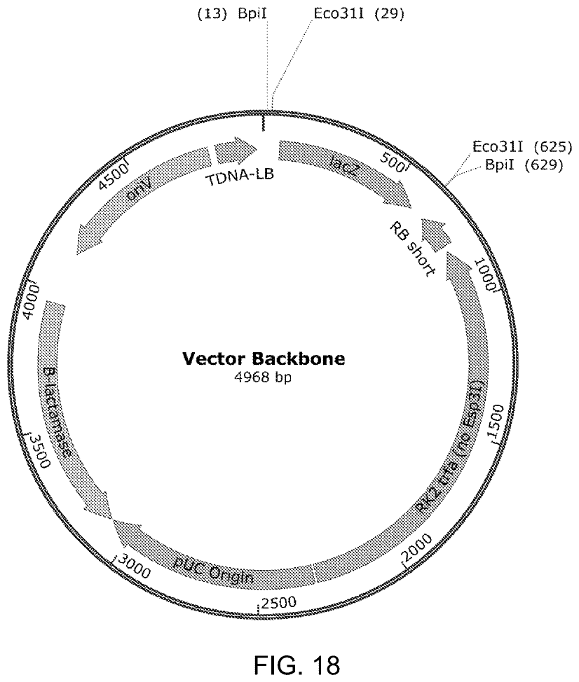

Referring now to FIG. 11, therein is shown an example of a pMOZ vector backbone [SEQ ID NO:60], which is an example vector backbone used to construct expression plasmids for the production of casein and kinases in plants. As a specific example, FIG. 18 is a map of a vector backbone including a nucleic acid sequence encoding a β-lactamase conferring resistance to carbenicillin that allows for the plasmid to be selected for in E. coli, a first origin of replication (pUC ori) [SEQ ID NO:61] that allows for the plasmid to be propagated in E. coli, and a second origin of replication (oriV) [SEQ ID NO:62] that allows for the plasmid to be propagated in either E. coli or Agrobacterium. In this way, expression plasmids can be assembled using standard cloning methods in bacteria and then transferred to Agrobacterium for transformation into plants.

Continuing this example, the vector backbone further includes two Eco31I restriction sites that allow for cloning of a single expression cassette into the vector backbone using standard GoldenGate or MoClo methods, an identification nucleic acid sequence encoding the lacZ gene (lacZ) [SEQ ID NO:63] to aid in the identification of correct clones through E. coli colony blue/white screening. The Eco31I sites are flanked by a left border repeat (LB) and a right border repeat (RB) from nopaline C58 T-DNA [SEQ ID NO:64] that are recognized by Agrobacterium and allow for an expression cassette to be transformed into plant cells and integrated into the plant host genome.

Referring now to FIG. 11, therein is shown an example of an expression plasmid for the production of kinase proteins in plants constructed using the vector backbone using standard GoldenGate or MoClo methods. As a specific example, FIG. 11 is a map of a pMOZ11 expression plasmid, which is an example expression plasmid for the production of recombinant H. sapiens Fam20C in plants. Continuing this example, the pMOZ11 expression plasmid includes a 1674 bp nucleic acid sequence encoding H. sapiens Fam20C (HsFam20C) [SEQ ID NO:66] that had been synthesized using only coding sequences from the original H. sapiens Fam20C gene and was codon optimized for expression in plants using Nicotiana benthamiana as the model expression system, operatively linked to a 426 bp sequence encoding the promoter Cauliflower Mosaic Virus 35S promoter (CaMV 35S Promoter) [SEQ ID NO: 42], a 62 bp translational enhancer encoding the omega leader sequence of the Tobacco Mosaic Virus (Ω TMV 5UTR) [SEQ ID NO: 68], a 57 bp nucleic acid sequence encoding the Arabidopsis thaliana ARA12 signal peptide gene (ARA12 Signal Peptide) [SEQ ID NO: 71] for localizing the recombinant protein to the endoplasmic reticulum of the plant cell, and a 15 bp sequence coding for the endoplasmic reticulum retention peptide HDEL [SEQ ID NO: 65]. PMOZ11 was assembled using the enzyme Eco31I and standard GoldenGate protocols. The resulting plasmid was transformed into E. coli and later purified using a standard miniprep protocol. Sanger sequencing was used to verify the correct assembly of the plasmid.

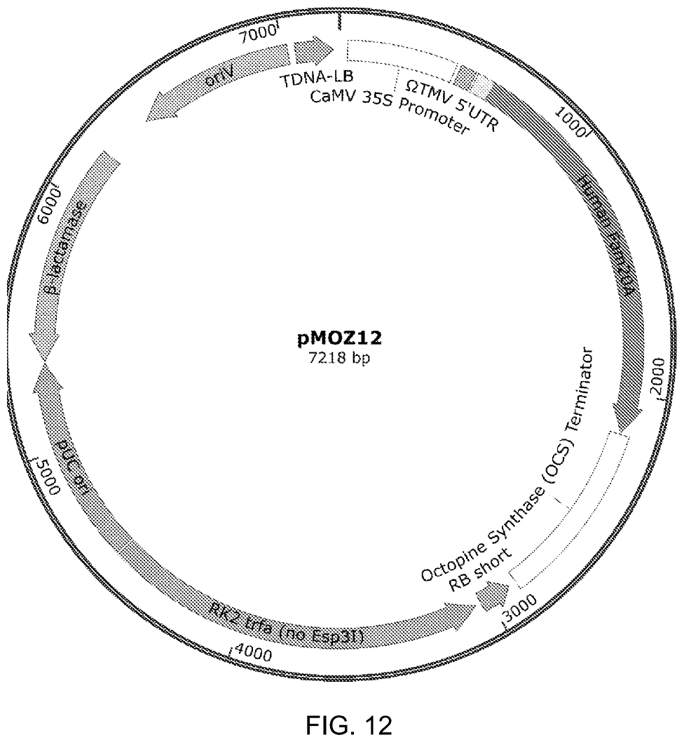

As another specific example, FIG. 12 is a map of a pMOZ12 expression plasmid, which is an example expression plasmid for the production of recombinant H. sapiens Fam20A in plants. Continuing this example, the pMOZ12 expression plasmid includes a 1524 bp nucleic acid sequence encoding H. sapiens Fam20A (HsFam20A) [SEQ ID NO:67] that had been synthesized using only coding sequences from the original H. sapiens Fam20C gene and was codon optimized for expression in plants using Nicotiana benthamiana as the model expression system, operatively linked to a 426 bp sequence encoding the promoter Cauliflower Mosaic Virus 35S promoter (CaMV 35S Promoter) [SEQ ID NO:42], a 62 bp translational enhancer encoding the omega leader sequence of the Tobacco Mosaic Virus (Ω TMV 5UTR) [SEQ ID NO:68], a 57 bp nucleic acid sequence encoding the Arabidopsis thaliana ARA12 signal peptide gene (ARA12 Signal Peptide) [SEQ ID NO:71] for localizing the recombinant protein to the endoplasmic reticulum of the plant cell, and a 15 bp sequence coding for the endoplasmic reticulum retention peptide HDEL [SEQ ID NO:65]. PMOZ12 was assembled using the enzyme Eco31I and standard GoldenGate protocols. The resulting plasmid was transformed into E. coli and later purified using a standard miniprep protocol. Sanger sequencing was used to verify the correct assembly of the plasmid.

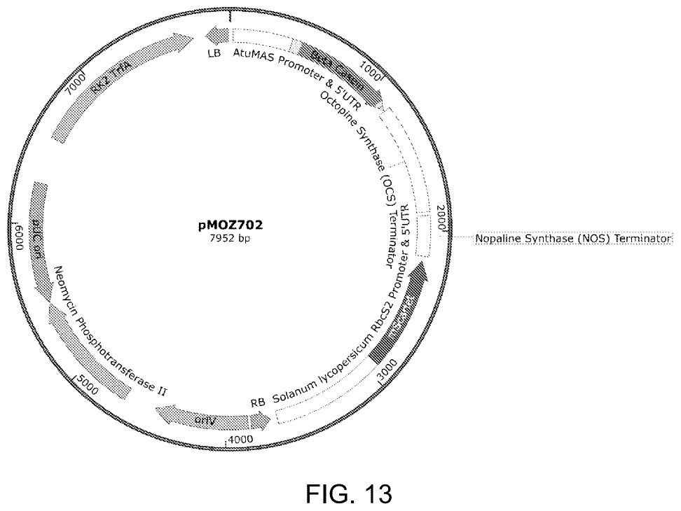

As another specific example, FIG. 13 is a map of a pMOZ702 expression plasmid, which is an example expression plasmid for the production of recombinant B. taurus Beta Casein in plants. Continuing this example, the pMOZ702 expression plasmid includes a 627 bp nucleic acid sequence encoding B. taurus β-casein [SEQ ID NO:77] that had been synthesized and was codon optimized for expression in plants using Nicotiana benthamiana as the model expression system, operatively linked to a 383 bp sequence encoding the promoter A. tumefaciens Mannopine Synthase promoter (AtuMAS Promoter) [SEQ ID NO:70], a 63 bp nucleic acid sequence encoding the Arabidopsis thaliana S2S signal peptide (AtS2S Signal Peptide) [SEQ ID NO:72] for localizing the recombinant protein to the endoplasmic reticulum of the plant cell, and a 63 bp sequence coding for an HA peptide tag, a 6× histidine peptide tag, and an endoplasmic reticulum retention peptide HDEL [SEQ ID NO:73]. These sequences code for a protein containing a signal peptide, beta casein, HA tag, 6 histidine tag, and HDEL peptide [SEQ ID NO:81]. The plasmid also contains a 732 bp nucleic acid sequence coding for the mScarlet fluorescent protein [SEQ ID NO: 75] that had been synthesized and was codon optimized for expression in plants using Nicotiana benthamiana as the model expression system, operatively linked to a 739 bp sequence encoding the S. lycopersicum RbcS2 promoter (SlRbcS2 Promoter) [SEQ ID NO: 59], and a 263 bp sequence encoding the A. tumefaciens Nopaline Synthase Terminator (NOS Terminator) [SEQ ID NO: 76]. pMOZ702 was assembled using the enzyme BpiI and standard GoldenGate protocols. The resulting plasmid was transformed into E. coli and later purified using a standard miniprep protocol. Sanger sequencing was used to verify the correct assembly of the plasmid.

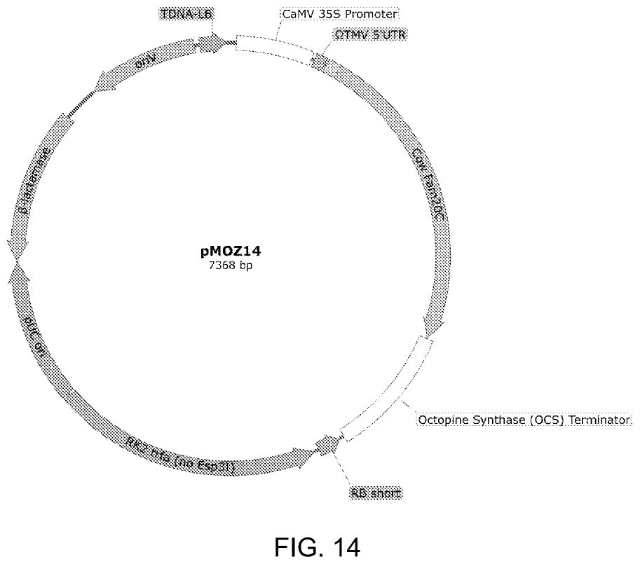

As another specific example, FIG. 14 is a map of a pMOZ14 expression plasmid, which is an example expression plasmid for the production of recombinant B. taurus Fam20C in plants. Continuing this example, the pMOZ14 expression plasmid includes a 1674 bp nucleic acid sequence encoding B. taurus Fam20C (BtFam20C) [SEQ ID NO:68] that had been synthesized using only coding sequences from the original B. taurus Fam20C gene and was codon optimized for expression in plants using Nicotiana benthamiana as the model expression system, operatively linked to a 426 bp sequence encoding the promoter Cauliflower Mosaic Virus 35S promoter (CaMV 35S Promoter) [SEQ ID NO:42], a 62 bp translational enhancer encoding the omega leader sequence of the Tobacco Mosaic Virus (Ω TMV 5UTR) [SEQ ID NO:58], a 57 bp nucleic acid sequence encoding the Arabidopsis thaliana ARA12 signal peptide gene (ARA12 Signal Peptide) [SEQ ID NO:71] for localizing the recombinant protein to the endoplasmic reticulum of the plant cell, and a 15 bp sequence coding for the endoplasmic reticulum retention peptide HDEL [SEQ ID NO:65]. PMOZ14 was assembled using the enzyme Eco31I and standard GoldenGate protocols. The resulting plasmid was transformed into E. coli and later purified using a standard miniprep protocol. Sanger sequencing was used to verify the correct assembly of the plasmid.

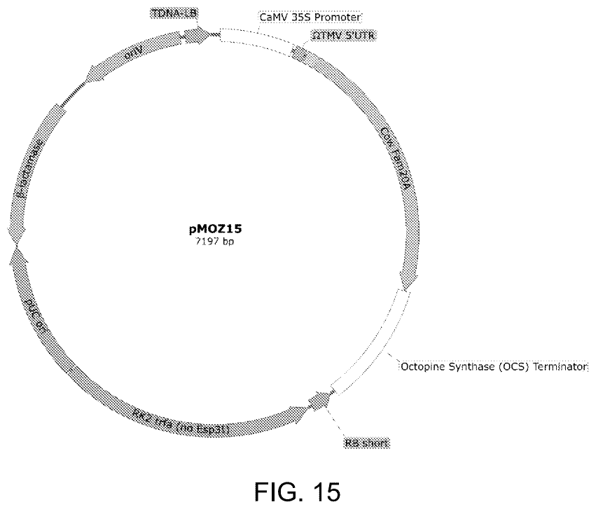

As another specific example, FIG. 15 is a map of a pMOZ15 expression plasmid, which is an example expression plasmid for the production of recombinant B. taurus Fam20A in plants. Continuing this example, the pMOZ15 expression plasmid includes a 1503 bp nucleic acid sequence encoding B. taurus Fam20A (BtFam20A) [SEQ ID NO:69] that had been synthesized using only coding sequences from the original B. taurus Fam20A gene and was codon optimized for expression in plants using Nicotiana benthamiana as the model expression system, operatively linked to a 426 bp sequence encoding the promoter Cauliflower Mosaic Virus 35S promoter (CaMV 35S Promoter) [SEQ ID NO:42], a 62 bp translational enhancer encoding the omega leader sequence of the Tobacco Mosaic Virus (Ω TMV 5UTR) [SEQ ID NO:58], a 57 bp nucleic acid sequence encoding the Arabidopsis thaliana ARA12 signal peptide gene (ARA12 Signal Peptide) [SEQ ID NO:71] for localizing the recombinant protein to the endoplasmic reticulum of the plant cell, and a 15 bp sequence coding for the endoplasmic reticulum retention peptide HDEL [SEQ ID NO:65]. PMOZ15 was assembled using the enzyme Eco31I and standard GoldenGate protocols. The resulting plasmid was transformed into E. coli and later purified using a standard miniprep protocol. Sanger sequencing was used to verify the correct assembly of the plasmid.

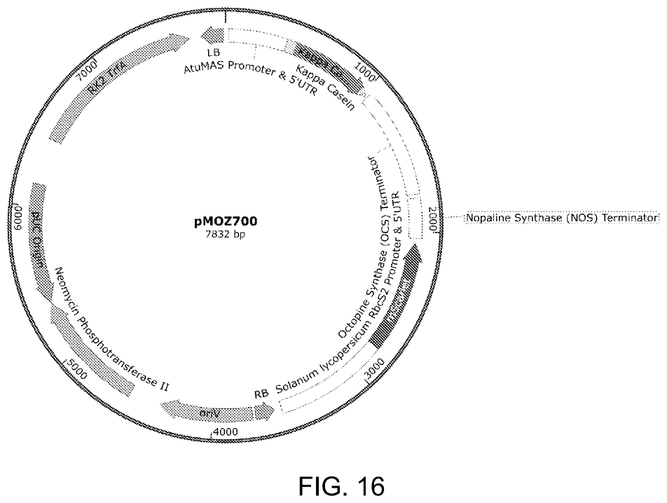

As another specific example, FIG. 16 is a map of a pMOZ700 expression plasmid, which is an example expression plasmid for the production of recombinant B. taurus Kappa Casein in plants. Continuing this example, the pMOZ700 expression plasmid includes a 507 bp nucleic acid sequence encoding B. taurus K-casein [SEQ ID NO:78] that had been synthesized and was codon optimized for expression in plants using Nicotiana benthamiana as the model expression system, operatively linked to a 383 bp sequence encoding the promoter A. tumefaciens Mannopine Synthase promoter (AtuMAS Promoter) [SEQ ID NO:70], a 63 bp nucleic acid sequence encoding the Arabidopsis thaliana S2S signal peptide (AtS2S Signal Peptide) [SEQ ID NO:72] for localizing the recombinant protein to the endoplasmic reticulum of the plant cell, and a 63 bp sequence coding for an HA peptide tag, a 6× histidine peptide tag, and an endoplasmic reticulum retention peptide HDEL [SEQ ID NO:73]. These sequences code for a protein containing a signal peptide, beta casein, HA tag, 6 histidine tag, and HDEL peptide [SEQ ID NO:82]. The plasmid also contains a 732 bp nucleic acid sequence coding for the mScarlet fluorescent protein [SEQ ID NO: 75] that had been synthesized and was codon optimized for expression in plants using Nicotiana benthamiana as the model expression system, operatively linked to a 739 bp sequence encoding the S. lycopersicum RbcS2 promoter (SlRbcS2 Promoter) [SEQ ID NO: 59], and a 263 bp sequence encoding the A. tumefaciens Nopaline Synthase Terminator (NOS Terminator) [SEQ ID NO: 76]. pMOZ702 was assembled using the enzyme BpiI and standard GoldenGate protocols. The resulting plasmid was transformed into E. coli and later purified using a standard miniprep protocol. Sanger sequencing was used to verify the correct assembly of the plasmid.

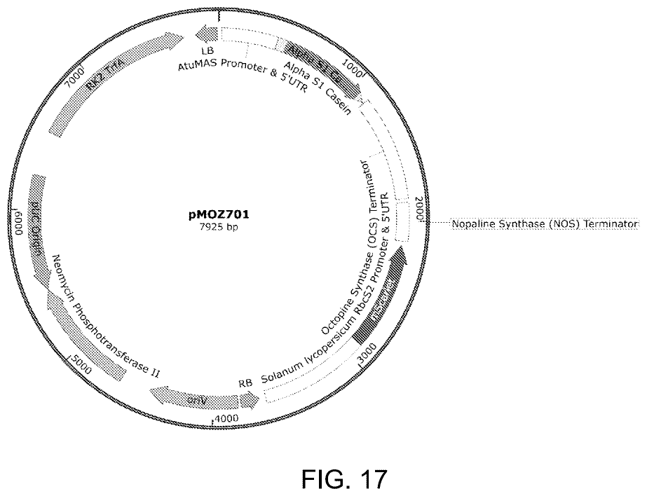

As another specific example, FIG. 17 is a map of a pMOZ701 expression plasmid, which is an example expression plasmid for the production of recombinant B. taurus αS1 Casein in plants. Continuing this example, the pMOZ701 expression plasmid includes a 597 bp nucleic acid sequence encoding B. taurus αS1-casein [SEQ ID NO:79] that had been synthesized and was codon optimized for expression in plants using Nicotiana benthamiana as the model expression system, operatively linked to a 383 bp sequence encoding the promoter A. tumefaciens Mannopine Synthase promoter (AtuMAS Promoter) [SEQ ID NO:70], a 63 bp nucleic acid sequence encoding the Arabidopsis thaliana S2S signal peptide (AtS2S Signal Peptide) [SEQ ID NO:72] for localizing the recombinant protein to the endoplasmic reticulum of the plant cell, and a 63 bp sequence coding for an HA peptide tag, a 6× histidine peptide tag, and an endoplasmic reticulum retention peptide HDEL [SEQ ID NO:73]. These sequences code for a protein containing a signal peptide, beta casein, HA tag, 6 histidine tag, and HDEL peptide [SEQ ID NO:45]. The plasmid also contains a 732 bp nucleic acid sequence coding for the mScarlet fluorescent protein [SEQ ID NO: 75] that had been synthesized and was codon optimized for expression in plants using Nicotiana benthamiana as the model expression system, operatively linked to a 739 bp sequence encoding the S. lycopersicum RbcS2 promoter (SlRbcS2 Promoter) [SEQ ID NO: 59], and a 263 bp sequence encoding the A. tumefaciens Nopaline Synthase Terminator (NOS Terminator) [SEQ ID NO: 76]. pMOZ701 was assembled using the enzyme BpiI and standard GoldenGate protocols. The resulting plasmid was transformed into E. coli and later purified using a standard miniprep protocol. Sanger sequencing was used to verify the correct assembly of the plasmid.

### Coexpression of 1 Casein and 2 Human Kinases

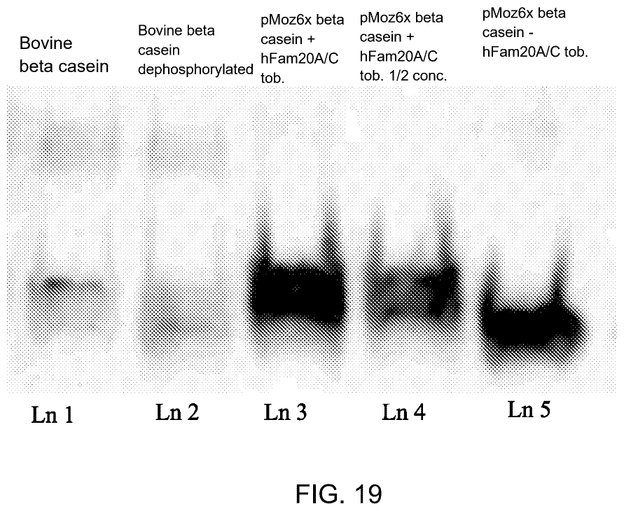

Referring to FIG. 19, therein is shown an example of the expression of recombinant Beta casein, HsFam20C, and HsFam20A in a systemically-infected N. benthamiana plant using combinations of the pMOZ11 (expresses HsFam20C), pMOZ12 (expresses HsFam20A) and pMOZ702 (expresses beta casein) expression plasmids. In this example, N. benthamiana plants were incubated in a growth room at 25° C. with a 16 hour light 8 hour dark cycle for 4 weeks. The 4-week old N. benthamiana plants were infiltrated with A. tumefaciens strain GV3101 carrying combinations of pMOZ plasmids.

In one condition the plants were infiltrated with three different cultures of A. tumefaciens strain GV3101 carrying pMOZ702, pMOZ11, and pMOZ12 all grown to an OD600 of 0.1.

In a second condition the plants were infiltrated with three different cultures of A. tumefaciens strain GV3101 carrying pMOZ702, pMOZ11, and pMOZ12 all grown to an OD600 of 0.05.

In a third condition the plants were infiltrated with one culture of A. tumefaciens strain GV3101 carrying pMOZ702 grown to an OD600 of 0.1.

Following vacuum infiltration, plants were blot-dried and returned to the growth room for 72 hours before being imaged. Infected leaves were imaged with an epifluorescent microscope with Red Fluorescent Protein (RFP) excitation and emission filters to confirm that the mScarlet protein from pMOZ702 was successfully expressing in the plant cells. Leaves that were expressing mScarlet were harvested, frozen with liquid nitrogen, crushed with a mortar and pestle. 250 mg of crushed plant tissue was transferred to a 1.7 mL tube and 300 μL of protein extraction buffer (800 uL of 500 mM sodium phosphate, 200 μL of 500 mM sodium phosphate dibasic, 1 mL 200 mM Sodium metabisulfite, 50 uL Tween-20, 5 mL 1M Trehalose, 3 mL diH2O) was added. This mixture was incubated on a rotisserie at 4 C for 1 hour and then centrifuged at 400RPM in with an Eppendorf 5415R centrifuge to pellet the solid plant material. The supernatant containing the extracted protein was transferred to a new 1.7 mL tube.

Further continuing this example, protein samples from the infected plant tissue were analyzed with sodium dodecyl sulfate polyacrylamide gel electrophoresis (SDS PAGE). 10 uL of supernatant was mixed with 30 uL of protein loading buffer (900 uL of 4× Laemmli Sample Buffer [Bio-Rad Laboratories]+100 uL of 2-mercaptoethanol) and heated for 5 minutes at 95 C. These samples were loaded onto Bio-Rad “Any kD” precast polyacrylamide gels along with a standard protein ladder and phosphorylated and dephosphorylated beta casein samples from Sigma Aldrich. The gel was run in 1× Tris/Glycine/SDS Buffer (Bio-Rad Laboratories) at 150V for 45 minutes. The gel was removed from the gel box and placed in a PVDF Transfer Pack (Bio-Rad Laboratories), the transfer pack was placed in a Trans-Blot Turbo (Bio-Rad Laboratories) and the proteins were transferred to the PVDF membrane using the “Mini TGX” settings. The PVDF membrane containing the transferred proteins was first washed in 25 mL Protein Free Blocking Buffer (ThermoFisher) for 1 hour, then incubated with 5 mL of Protein Free Blocking Buffer containing 5 uL of anti-beta-casein polyclonal rabbit IgG at room temperature for 4 hours. The membrane was then washed three times with 10 mL TBST (20 mM Tris-HCl, 150 mM NaCl, 0.1% Tween 20) for 10 minutes at room temperature. Then the membrane was washed with 25 mL of Protein Free Blocking Buffer containing 2 μL of anti-rabbit IgG secondary antibody conjugated to horseradish peroxidase for 1 hour at room temperature and then poured off. The membrane was placed in a ChemiDoc MP imaging system (Bio-Rad Laboratories) and 1 mL of SuperSignal West Pico (ThermoFisher) luminescent imaging solution was added to the membrane. Images were captured using the Chemiluminescence setting on the ChemiDoc MP.

Further continuing this example, the casein proteins expressed in plant cells show up on the anti-beta-casein Western blot with varying migration distances (FIG. 19). Lane 1 purified beta casein standard. Lane 2: dephosphorylated beta casein from milk standard. Lane 3: Beta casein+human Fam20C+human Fam20A expressed in tobacco leaf (Tobacco leaf tissue transformed with pMOZ702+pMOZl 1+pMOZ12). Lane 4: same as lane 3, with half as much protein loaded on gel. Lane 5: Beta casein (pMOZ702) expressed in tobacco leaf (no kinase). In lanes containing beta casein coexpressed with the human kinases the bands are shifted upward on the gel relative to the sample transformed with only beta casein, suggesting that the molecular weight of the beta casein has increased due to phosphorylation.

### Coexpression of 1 Casein and 1 or 2 Kinases

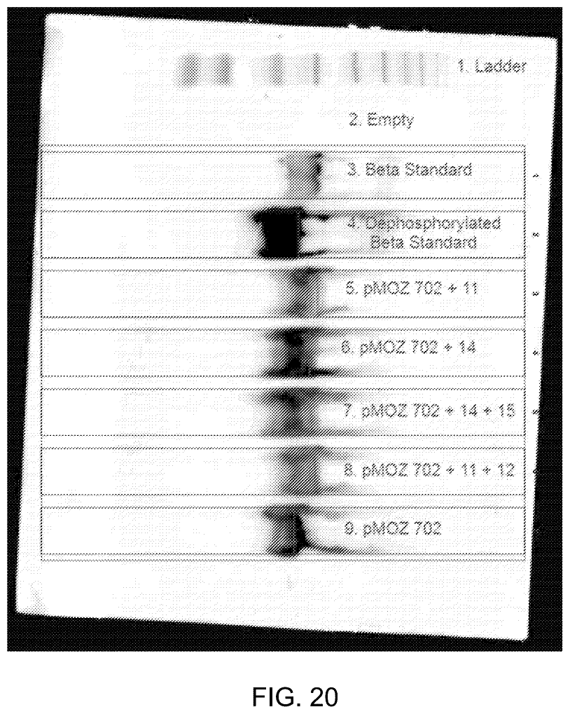

Referring to FIG. 20, therein is shown an example of the expression of recombinant Beta casein, HsFam20C, and HsFam20A in a systemically-infected N. benthamiana plant using combinations of the pMOZ11 (expresses HsFam20C), pMOZ12 (expresses HsFam20A), pMOZ14 (expresses BtFam20C), pMOZ15 (expresses BtFam20A) and pMOZ702 (expresses beta casein) expression plasmids. In this example, N. benthamiana plants were incubated in a growth room at 25° C. with a 16 hour light 8 hour dark cycle for 4 weeks. The 4-week old N. benthamiana plants were infiltrated with A. tumefaciens strain GV3101 carrying combinations of pMOZ plasmids at an OD600 of 0.1.

In one condition the plants were infiltrated with two different cultures of A. tumefaciens strain GV3101 carrying pMOZ702, pMOZ11.

In a second condition the plants were infiltrated with three different cultures of A. tumefaciens strain GV3101 carrying pMOZ702, pMOZ11, and pMOZ12.

In a third condition the plants were infiltrated with a single culture of A. tumefaciens strain GV3101 carrying pMOZ702.

In a fourth condition the plants were infiltrated with two different cultures of A. tumefaciens strain GV3101 carrying pMOZ702, pMOZ14.

In a fifth condition the plants were infiltrated with three different cultures of A. tumefaciens strain GV3101 carrying pMOZ702, pMOZ14, and pMOZ15.

Following vacuum infiltration, plants were blot-dried and returned to the growth room for 72 hours before being imaged. Infected leaves were imaged with an epifluorescent microscope with Red Fluorescent Protein (RFP) excitation and emission filters to confirm that the mScarlet protein from pMOZ702 was successfully expressing in the plant cells. Leaves that were expressing mScarlet were harvested, frozen with liquid nitrogen, crushed with a mortar and pestle. 250 mg of crushed plant tissue was transferred to a 1.7 mL tube and 300 μL of protein extraction buffer (800 uL of 500 mM sodium phosphate, 200 μL of 500 mM sodium phosphate dibasic, 1 mL 200 mM Sodium metabisulfite, 50 uL Tween-20, 5 mL 1M Trehalose, 3 mL diH2O) was added. This mixture was incubated on a rotisserie at 4 C for 1 hour and then centrifuged at 400RPM in with an Eppendorf 5415R centrifuge to pellet the solid plant material. The supernatant containing the extracted protein was transferred to a new 1.7 mL tube.

Further continuing this example, protein samples from the infected plant tissue were analyzed with sodium dodecyl sulfate polyacrylamide gel electrophoresis (SDS PAGE). 10 uL of supernatant was mixed with 30 μL of protein loading buffer (900 uL of 4× Laemmli Sample Buffer [Bio-Rad Laboratories]+100 μL of 2-mercaptoethanol) and heated for 5 minutes at 95 C. These samples were loaded onto Bio-Rad “Any kD” precast polyacrylamide gels along with a standard protein ladder and phosphorylated and dephosphorylated beta casein samples from Sigma Aldrich. The gel was run in 1× Tris/Glycine/SDS Buffer (Bio-Rad Laboratories) at 150V for 45 minutes. The gel was removed from the gel box and placed in a PVDF Transfer Pack (Bio-Rad Laboratories), the transfer pack was placed in a Trans-Blot Turbo (Bio-Rad Laboratories) and the proteins were transferred to the PVDF membrane using the “Mini TGX” settings. The PVDF membrane containing the transferred proteins was first washed in 25 mL Protein Free Blocking Buffer (ThermoFisher) for 1 hour, then incubated with 5 mL of Protein Free Blocking Buffer containing 5 μL of anti-beta-casein polyclonal rabbit IgG at room temperature for 4 hours. The membrane was then washed three times with 10 mL TBST (20 mM Tris-HCl, 150 mM NaCl, 0.1% Tween 20) for 10 minutes at room temperature. Then the membrane was washed with 25 mL of Protein Free Blocking Buffer containing 2 uL of anti-rabbit IgG secondary antibody conjugated to horseradish peroxidase for 1 hour at room temperature and then poured off. The membrane was placed in a ChemiDoc MP imaging system (Bio-Rad Laboratories) and 1 mL of SuperSignal West Pico (ThermoFisher) luminescent imaging solution was added to the membrane. Images were captured using the Chemiluminescence setting on the ChemiDoc MP.

Further continuing this example, the casein proteins expressed in plant cells show up on the anti-beta-casein Western blot with varying migration distances (FIG. 20). Lane 1: Molecular weight ladder. Lane 2: Empty. Lane 3: Purified beta casein from milk as standard. Lane 4: dephosphorylated beta casein from milk as standard. Lane 5: beta casein+human Fam20C expressed in tobacco leaf (Tobacco leaf tissue transformed with pMOZ702 and pMOZ11). Lane 6: beta casein+cow Fam20C expressed in tobacco leaf (Tobacco leaf tissue transformed with pMOZ702 and pMOZ14). Lane 7: beta casein+cow Fam20C+cow Fam20A expressed in tobacco leaf (Tobacco leaf tissue transformed with pMOZ702, pMOZ14, and pMOZ15). Lane 8: beta casein+human Fam20C+human Fam20A expressed in tobacco leaf (Tobacco leaf tissue transformed with pMOZ702, pMOZl 1, and pMOZ12). Lane 9: Tobacco leaf tissue transformed with only pMOZ702. In lanes containing beta casein coexpressed with 1 human kinase, 2 human kinases, 1 bovine kinase or 2 bovine kinases the bands are shifted upward on the gel relative to the sample transformed with only beta casein, suggesting that the molecular weight of the beta casein has increased due to phosphorylation.

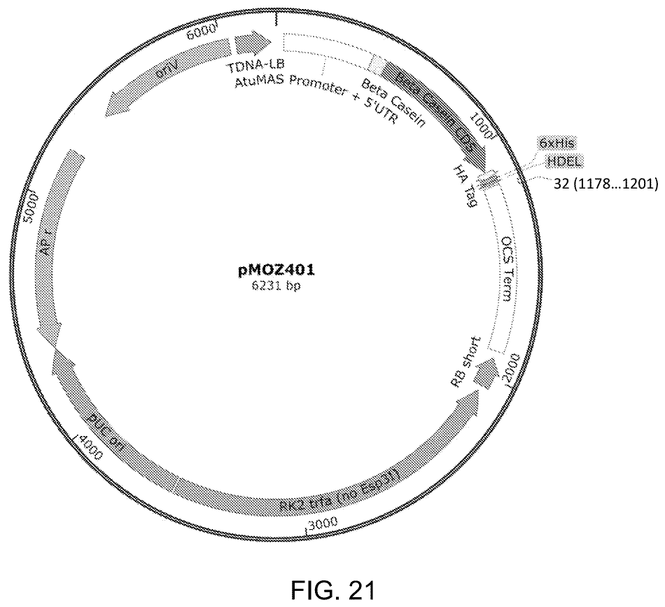

As another specific example, FIG. 21 is a map of a pMOZ401 expression plasmid, which is an example expression plasmid for the production of recombinant B. taurus Beta Casein in plants. Continuing this example, the pMOZ401 expression plasmid includes a 627 bp nucleic acid sequence encoding B. taurus β-casein [SEQ ID NO:77] that had been synthesized and was codon optimized for expression in plants using Nicotiana benthamiana as the model expression system, operatively linked to a 383 bp sequence encoding the promoter A. tumefaciens Mannopine Synthase promoter (AtuMAS Promoter) [SEQ ID NO:70], a 63 bp nucleic acid sequence encoding the Arabidopsis thaliana S2S signal peptide (AtS2S Signal Peptide) [SEQ ID NO:72] for localizing the recombinant protein to the endoplasmic reticulum of the plant cell, and a 63 bp sequence coding for an HA peptide tag, a 6× histidine peptide tag, and an endoplasmic reticulum retention peptide HDEL [SEQ ID NO:73]. These sequences code for a protein containing a signal peptide, beta casein, HA tag, 6 histidine tag, and HDEL peptide [SEQ ID NO:81]. pMOZ401 was assembled using the enzyme Eco3ll and standard GoldenGate protocols. The resulting plasmid was transformed into E. coli and later purified using a standard mini prep protocol. Sanger sequencing was used to verify the correct assembly of the plasmid.

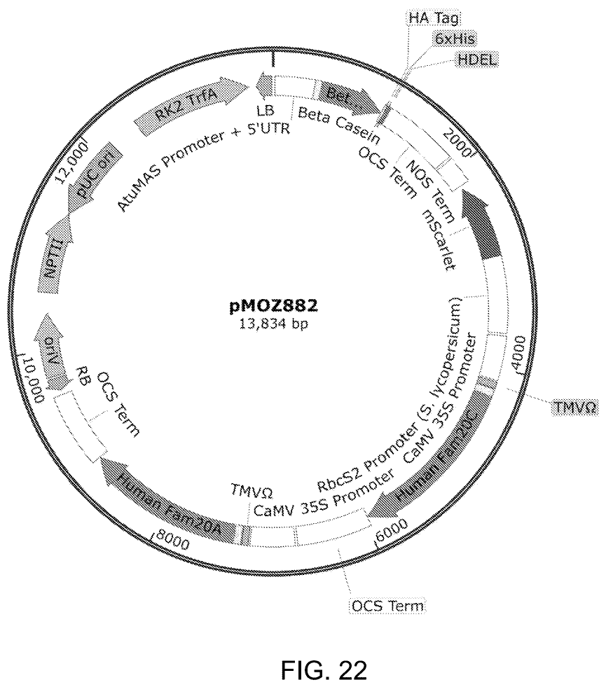

As another specific example, FIG. 22 is a map of a pMOZ882 expression plasmid, which is an example expression plasmid for the production of recombinant B. taurus Beta Casein in plants. Continuing this example, the pMOZ882 expression plasmid includes a 627 bp nucleic acid sequence encoding B. taurus β-casein [SEQ ID NO:77] that had been synthesized and was codon optimized for expression in plants using Nicotiana benthamiana as the model expression system, operatively linked to a 383 bp sequence encoding the promoter A. tumefaciens Mannopine Synthase promoter (AtuMAS Promoter) [SEQ ID NO:70], a 63 bp nucleic acid sequence encoding the Arabidopsis thaliana S2S signal peptide (AtS2S Signal Peptide) [SEQ ID NO:72] for localizing the recombinant protein to the endoplasmic reticulum of the plant cell, and a 63 bp sequence coding for an HA peptide tag, a 6× histidine peptide tag, and an endoplasmic reticulum retention peptide HDEL [SEQ ID NO:73]. These sequences code for a protein containing a signal peptide, beta casein, HA tag, 6 histidine tag, and HDEL peptide [SEQ ID NO:81]. The plasmid also contains a 732 bp nucleic acid sequence coding for the mScarlet fluorescent protein [SEQ ID NO: 75] that had been synthesized and was codon optimized for expression in plants using Nicotiana benthamiana as the model expression system, operatively linked to a 739 bp sequence encoding the S. lycopersicum RbcS2 promoter (SlRbcS2 Promoter) [SEQ ID NO: 59], and a 263 bp sequence encoding the A. tumefaciens Nopaline Synthase Terminator (NOS Terminator) [SEQ ID NO: 76]. The plasmid also contains a 1674 bp nucleic acid sequence encoding H. sapiens Fam20C (HsFam20C) [SEQ ID NO:66] that had been synthesized using only coding sequences from the original H. sapiens Fam20C gene and was codon optimized for expression in plants using Nicotiana benthamiana as the model expression system, operatively linked to a 426 bp sequence encoding the promoter Cauliflower Mosaic Virus 35S promoter (CaMV 35S Promoter) [SEQ ID NO:42], a 62 bp translational enhancer encoding the omega leader sequence of the Tobacco Mosaic Virus (Q TMV 5UTR) [SEQ ID NO: 58], a 57 bp nucleic acid sequence encoding the Arabidopsis thaliana ARA12 signal peptide gene (ARA12 Signal Peptide) [SEQ ID NO:71] for localizing the recombinant protein to the endoplasmic reticulum of the plant cell, and a 15 bp sequence coding for the endoplasmic reticulum retention peptide HDEL [SEQ ID NO: 65]. The plasmid also contains a 1524 bp nucleic acid sequence encoding H. sapiens Fam20A (HsFam20A) [SEQ ID NO:67] that had been synthesized using only coding sequences from the original H. sapiens Fam20A gene and was codon optimized for expression in plants using Nicotiana benthamiana as the model expression system, operatively linked to a 426 bp sequence encoding the promoter Cauliflower Mosaic Virus 35S promoter (CaMV 35S Promoter) [SEQ ID NO:42], a 62 bp translational enhancer encoding the omega leader sequence of the Tobacco Mosaic Virus (Q TMV 5UTR) [SEQ ID NO: 58], a 57 bp nucleic acid sequence encoding the Arabidopsis thaliana ARA12 signal peptide gene (ARA12 Signal Peptide) [SEQ ID NO:71] for localizing the recombinant protein to the endoplasmic reticulum of the plant cell, and a 15 bp sequence coding for the endoplasmic reticulum retention peptide HDEL [SEQ ID NO: 65]. pMOZ882 was assembled using the enzyme BpiI and standard GoldenGate protocols. The resulting plasmid was transformed into E. coli and later purified using a standard mini prep protocol. Sanger sequencing was used to verify the correct assembly of the plasmid.

### Example 1. Phosphorylation Increased Casein Expression

In this proposed experiment the expression level of casein proteins is shown to increase when phosphorylated. Recombinant Kappa casein and BtFam20C kinase are expressed in a systemically-infected N. benthamiana plant using combinations of the pMOZ14 (expresses BtFam20C) and pMOZ700 (expresses bovine kappa casein) expression plasmids. In this example, N. benthamiana plants were incubated in a growth room at 25° C. with a 16 hour light 8 hour dark cycle for 4 weeks. The 4-week old N. benthamiana plants were infiltrated with A. tumefaciens strain GV3101 carrying combinations of pMOZ plasmids.

In one condition the N. benthamiana plants were infiltrated with two different cultures of A. tumefaciens strain GV3101 carrying pMOZ700, pMOZ14 all grown to an OD600 of 0.1.

In a second condition N. benthamiana plants were infiltrated with a single culture of A. tumefaciens strain GV3101 carrying pMOZ700 grown to an OD600 of 0.1.

Following vacuum infiltration, plants will be blot-dried and returned to the growth room for 72 hours before being imaged. Infected leaves will be imaged with an epifluorescent microscope with Red Fluorescent Protein (RFP) excitation and emission filters to confirm that the mScarlet protein from pMOZ700 was successfully expressed in the plant cells. Leaves that express mScarlet will be harvested, frozen with liquid nitrogen, crushed with a mortar and pestle. 250 mg of crushed plant tissue will be weighed and transferred to a 1.7 mL tube and 300 μL of protein extraction buffer (800 uL of 500 mM sodium phosphate, 200 μL of 500 mM sodium phosphate dibasic, 1 mL 200 mM Sodium metabisulfite, 50 uL Tween-20, 5 mL 1M Trehalose, 3 mL diH2O) will be added. This mixture will be incubated on a rotisserie at 4 C for 1 hour and then centrifuged to pellet the solid plant material. The supernatant containing the extracted protein will be transferred to a new tube.

Continuing this example, extracted protein will be analyzed with SDS PAGE followed by a Western blot with kappa casein antibodies using a typical Western blotting protocol. After incubating with an appropriate secondary antibody conjugated to horseradish peroxidase, luminescent developing solution such as SuperSignal West (ThermoFisher) will be applied to the Western membrane and imaged on a ChemiDoc MP (Bio-Rad Laboratories) imaging system. The brightness of the kappa casein bands will be quantified using functions built into the ChemiDoc MP. Brighter bands in lanes containing protein from the plants transformed with both pMOZ700 (Kappa casein) and pMOZ14 (kinase) compared to protein from plants transformed with only pMOZ700 (kappa casein) shows that phosphorylated casein proteins are expressed at higher concentrations than non-phosphorylated casein.

In a similar experiment, N. benthamiana plants will be transformed with the same casein and kinase plasmids and protein will be extracted the same as just described. Extracted protein supernatants will be analyzed by high pressure liquid chromatography (HPLC). The supernatants will be diluted with acetate buffer and loaded into the HPLC apparatus. Eluted protein will be detected and quantified by UV absorption. Integrals will be calculated for the peaks corresponding to casein proteins to quantify the concentration of casein in each sample.

Larger integral values for casein proteins from plants transformed with casein and kinase compared to casein from plants transformed with only casein will show that phosphorylation of casein increases their expression level.

### Example 2. Phosphorylation Increased Aggregation of Casein Proteins

In this proposed experiment the aggregation of multiple caseins proteins is shown to be increased when the caseins are phosphorylated compared to non-phosphorylated caseins. Recombinant bovine alpha S1 casein casein, bovine beta casein and BtFam20C kinase will be expressed in a in a systemically-infected N. benthamiana plant using combinations of the pMOZ14 (expresses BtFam20C), pMOZ701 (expresses bovine alpha S1 casein), and pMOZ702 (expresses bovine beta casein) expression plasmids. In this example, N. benthamiana plants will be incubated in a growth room at 25° C. with a 16 hour light 8 hour dark cycle for 4 weeks. The 4-week old N. benthamiana plants will be infiltrated with A. tumefaciens strain GV3101 carrying combinations of pMOZ plasmids.

In one condition the N. benthamiana plants were infiltrated with two different cultures of A. tumefaciens strain GV3101 carrying pMOZ701 and pMOZ14 all grown to an OD600 of 0.1.

In a second condition the N. benthamiana plants were infiltrated with two different cultures of A. tumefaciens strain GV3101 carrying pMOZ702 and pMOZ14 all grown to an OD600 of 0.1.

In a third condition the N. benthamiana plants were infiltrated with a single culture of A. tumefaciens strain GV3101 carrying pMOZ701 grown to an OD600 of 0.1.

In a fourth condition the N. benthamiana plants were infiltrated with a single culture of A. tumefaciens strain GV3101 carrying pMOZ702 grown to an OD600 of 0.1.

Following vacuum infiltration, plants will be blot-dried and returned to the growth room for 72 hours before being imaged. Infected leaves will be imaged with an epifluorescent microscope with Red Fluorescent Protein (RFP) excitation and emission filters to confirm that the mScarlet protein from pMOZ701 or pMOZ702 was successfully expressed in the plant cells. Leaves that express mScarlet will be harvested, frozen with liquid nitrogen, crushed with a mortar and pestle. 250 mg of crushed plant tissue will be weighed and transferred to a 1.7 mL tube and 300 μL of protein extraction buffer (800 uL of 500 mM sodium phosphate, 200 μL of 500 mM sodium phosphate dibasic, 1 mL 200 mM Sodium metabisulfite, 50 uL Tween-20, 5 mL 1M Trehalose, 3 mL diH2O) will be added. This mixture will be incubated on a rotisserie at 4 C for 1 hour and then centrifuged to pellet the solid plant material. The supernatant containing the extracted protein will be transferred to a new tube for further analysis.

Continuing this example, various combinations of protein supernatants from the different transformation conditions will be used in a co-immunoprecipitation (CoIP) assay to measure the amount of protein-protein aggregation. In each CoIP assay protein supernatant from one sample will be mixed with magnetic anti-HA Dynabeads (ThermoFisher catalog #88837) so that the HA peptide tag attached to the casein protein expressed from either pMOZ701 or pMOZ702 plasmid is contacted with anti-HA antibodies attached to the magnetic beads. The quantity of protein added will be great enough to saturate all available HA antibodies on the surface of the beads. The HA-labeled casein proteins will stick to the magnetic beads and the rest of the supernatant will be washed away with wash buffer ((10 mM Tris pH 7.4, 1 mM EDTA, 1 mM EGTA. pH 8.0, 150 mM NaCl, 1% Triton X-100, 0.2 mM sodium orthovanadate) while the casein is retained by the magnetic beads held stationary by an external magnetic force. Protein supernatant from a second sample will then be contacted with the beads and allowed to incubate at room temperature for 1 hour, The supernatant will then be washed with wash buffer while the beads are held stationary by an external magnetic force. Any protein stuck the beads will then be released from the beads by adding 30 μL of Laemmli buffer (65.8 mM Tris-HC, pH 6.8, 2.1% SDS, 26.3% glycerol, 2% 2-mercaptoethanol) and incubating at 90 degrees celsius for 20 minutes. The beads will then be removed from the Laemmli buffer using a magnet and the remaining liquid will be analyzed by SDS PAGE and Western blot using standard protocols. The Western blot will be developed using a casein-specific antibody targeting the second casein protein that was added to the CoIP assay. Analyzing the brightness of the bands on the Western blot will show which samples captured more secondary casein protein.

In one condition, the supernatant from plants transformed with pMOZ701 will be first contacted with the beads and then supernatant from plants transformed with pMOZ702 will be contacted second.

In a second condition, the supernatant from plants transformed with pMOZ702 will be first contacted with the beads and then supernatant from plants transformed with pMOZ701 will be contacted second.

In a third condition, the supernatant from plants transformed with pMOZ702 and pMOZ14 will be first contacted with the beads and then supernatant from plants transformed with pMOZ701 and pMOZ14 will be contacted second.

In a third condition, the supernatant from plants transformed with pMOZ701 and pMOZ14 will be first contacted with the beads and then supernatant from plants transformed with pMOZ702 and pMOZ14 will be contacted second.

Further continuing this example, Western blots showing increased amounts of casein eluted from the beads from samples where both casein plasmids (pMOZ701 or pMOZ702) were co-transformed with kinase plasmids (pMOZ14) compared to samples where the casein plasmids were transformed without kinase plasmid will indicate that phosphorylation of caseins increases their ability to aggregate or bind to each other.

### Example 3. Phosphorylation Improves Casein Micelle Formation

In this proposed experiment micelles will form in vivo when casein is phosphorylated. Recombinant Beta casein, Kappa casein, Alpha S1 casein, and BtFam20C kinase are expressed in a in a systemically-infected N. benthamiana plant using combinations of the pMOZ14 (expresses BtFam20C), pMOZ702 (expresses bovine beta casein), pMOZ701 (expresses alpha casein), and pMOZ700 (expresses bovine kappa casein) expression plasmids. In this example, N. benthamiana plants were incubated in a growth room at 25° C. with a 16 hour light 8 hour dark cycle for 4 weeks. The 4-week old N. benthamiana plants were infiltrated with A. tumefaciens strain GV3101 carrying combinations of pMOZ plasmids.

In one condition the N. benthamiana plants were infiltrated with four different cultures of A. tumefaciens strain GV3101 carrying pMOZ700, pMOZ701, pMOZ702, and pMOZ14 all grown to an OD600 of 0.1.

In a second condition N. benthamiana plants were infiltrated with three different cultures of A. tumefaciens strain GV3101 carrying pMOZ700, pMOZ701, and pMOZ702 all grown to an OD600 of 0.1.

Following vacuum infiltration, plants will be blot-dried and returned to the growth room for 72 hours before being imaged. Infected leaves will be imaged with an epifluorescent microscope with Red Fluorescent Protein (RFP) excitation and emission filters to confirm that the mScarlet protein from pMOZ700 or pMOZ701 or pMOZ702 were successfully expressed in the plant cells. Leaves that express mScarlet will be cut from the plant and then fixed in formaldehyde and osmium tetroxide using standard fixation and clearing protocols. The fixed tissue will then be sectioned and imaged on a transmission electron microscope. Comparison of images of leaves transformed with and without pMOZ14 will show that casein micelles form when kinase is present to phosphorylate the casein protein.

### Example 4. Phosphorylation Increased Viscosity (Quality/Mouthfeel)

In this proposed experiment the amount of calcium bound to casein proteins is shown to be increased when casein is phosphorylated. Recombinant Kappa casein and BtFam20C kinase are expressed in a in a systemically-infected N. benthamiana plant using combinations of the pMOZ14 (expresses BtFam20C) and pMOZ702 (expresses bovine alpha casein) expression plasmids. In this example, N. benthamiana plants were incubated in a growth room at 25° C. with a 16 hour light 8 hour dark cycle for 4 weeks. The 4-week old N. benthamiana plants were infiltrated with A. tumefaciens strain GV3101 carrying combinations of pMOZ plasmids.

In one condition the N. benthamiana plants were infiltrated with two different cultures of A. tumefaciens strain GV3101 carrying pMOZ702, pMOZ14 all grown to an OD600 of 0.1.

In a second condition N. benthamiana plants were infiltrated with a single culture of A. tumefaciens strain GV3101 carrying pMOZ702 grown to an OD600 of 0.1.

Following vacuum infiltration, plants will be blot-dried and returned to the growth room for 72 hours before being imaged. Infected leaves will be imaged with an epifluorescent microscope with Red Fluorescent Protein (RFP) excitation and emission filters to confirm that the mScarlet protein from pMOZ700 was successfully expressed in the plant cells. Leaves that express mScarlet will be harvested, frozen with liquid nitrogen, crushed with a mortar and pestle. 250 mg of crushed plant tissue will be weighed and transferred to a 1.7 mL tube and 300 uL of protein extraction buffer (800 uL of 500 mM sodium phosphate, 200 uL of 500 mM sodium phosphate dibasic, 1 mL 200 mM Sodium metabisulfite, 50 uL Tween-20, 5 mL 1M Trehalose, 3 mL diH2O) will be added. This mixture will be incubated on a rotisserie at 4 C for 1 hour and then centrifuged to pellet the solid plant material. The supernatant containing the extracted protein will be transferred to a new tube.

Continuing this example, the viscosity of protein supernatants of samples transformed with and without the kinase encoded by pMOZ14 will be measured and compared. 10 uL of each supernatant will be loaded into RheoSense microVISC viscometer and the viscosities will be measured. Results showing higher values for samples co-transformed with pMOZ14 indicate that phosphorylation of caseins increases viscosity of solutions containing those casein proteins.

### Example 5. Phosphorylated Casein Increased Calcium Binding

In this proposed experiment the amount of calcium bound to casein proteins is shown to be increased when casein is phosphorylated. Recombinant Kappa casein and BtFam20C kinase are expressed in a in a systemically-infected N. benthamiana plant using combinations of the pMOZ14 (expresses BtFam20C) and pMOZ700 (expresses bovine kappa casein) expression plasmids. In this example, N. benthamiana plants were incubated in a growth room at 25° C. with a 16 hour light 8 hour dark cycle for 4 weeks. The 4-week old N. benthamiana plants were infiltrated with A. tumefaciens strain GV3101 carrying combinations of pMOZ plasmids.

In one condition the N. benthamiana plants were infiltrated with two different cultures of A. tumefaciens strain GV3101 carrying pMOZ700, pMOZ14 all grown to an OD600 of 0.1.

In a second condition N. benthamiana plants were infiltrated with a single culture of A. tumefaciens strain GV3101 carrying pMOZ700 grown to an OD600 of 0.1.

Following vacuum infiltration, plants will be blot-dried and returned to the growth room for 72 hours before being imaged. Infected leaves will be imaged with an epifluorescent microscope with Red Fluorescent Protein (RFP) excitation and emission filters to confirm that the mScarlet protein from pMOZ700 was successfully expressed in the plant cells. Leaves that express mScarlet will be harvested, frozen with liquid nitrogen, crushed with a mortar and pestle. 250 mg of crushed plant tissue will be weighed and transferred to a 1.7 mL tube and 300 uL of protein extraction buffer (800 uL of 500 mM sodium phosphate, 200 uL of 500 mM sodium phosphate dibasic, 1 mL 200 mM Sodium metabisulfite, 50 uL Tween-20, 5 mL 1M Trehalose, 3 mL diH2O) will be added. This mixture will be incubated on a rotisserie at 4 C for 1 hour and then centrifuged to pellet the solid plant material. The supernatant containing the extracted protein will be transferred to a new tube.

Continuing this example, protein supernatants will be assayed for calcium content by first purifying the protein using anti-HA magnetic beads, then by a colorimetric assay specific for calcium. Supernatant from either plants transformed with both pMOZ702 and pMOZ14 or plants only transformed with pMOZ702 will be mixed with magnetic anti-HA Dynabeads (ThermoFisher catalog #88837) so that the HA peptide tag attached to the casein protein expressed from either pMOZ702 plasmid is contacted with anti-HA antibodies attached to the magnetic beads. The HA-labeled casein proteins will stick to the magnetic beads and the rest of the supernatant will be washed away with wash buffer ((10 mM Tris pH 7.4, 1 mM EDTA, 1 mM EGTA, pH 8.0, 150 mM NaCl, 1% Triton X-100, 0.2 mM sodium orthovanadate) while the casein is retained by the magnetic beads held stationary by an external magnetic force. Any protein stuck to the beads will then be released from the beads by adding 30 uL Tris buffer (10 mM Tris-HCl, pH 7.5) and incubating at 90 degrees celsius for 20 minutes. The beads will then be removed from the Tris buffer using a magnet. The remaining liquid will be assayed for calcium concentration using a Calcium Assay Kit from Abcam (catalog #ab102505). The protocol provided with the kit will be followed and the color intensity will be read out by a colorimetric plate reader. The values will be compared to a standard curve to calculate calcium concentrations. Results showing increased calcium in samples where both pMOZ702 and pMOZ14 were transformed compared to samples where only pMOZ702 was transformed will indicate that phosphorylated caseins bind calcium to a greater degree than non-phosphorylated caseins.

### Example 6 Coexpression of Casein and Human Kinase in Soybean

Example 6 is an example of the expression of recombinant Beta casein, HsFam20A, and HsFam20C in Glycine max (l.) merr protoplasts isolated from immature cotyledon nodes using combinations of the pMOZl 1 (expresses HsFam20C), pMOZ702 (expresses beta casein), pMOZ401 (expresses beta casein), and pMOZ882 (expresses beta casein, HsFam20A and HsFam20C) expression plasmids. In this example, Glycine max (l.) merr plants are incubated in a growth room at 28° C. with a 12 hour light 12 hour dark cycle for about 4 weeks. Seeds are removed from the plant and dissected to reveal the cotyledon tissue. The cotyledons are incubated in an enzyme solution (7.5 g/L cellulase R-10, 5 g/L macerozyme R-10, 1.4 g/L calcium chloride, 1 g/L BSA, 116.75 g/L mannitol, 4.25 g/L MES, and 1.5 g/L potassium chloride) overnight to digest the cell walls and isolate the protoplasts. After incubating in enzyme solution overnight, the protoplasts are strained to remove undigested tissue, spun down for 3 minutes at 0.1 RCF and washed with CPW9M (27.2 mg/L potassium phosphate monobasic, 101 mg/L potassium nitrate, 1.47 g/L calcium chloride dihydrate, 246 mg/L magnesium sulfate heptahydrate, 100 μL of 1 mM stock/L of copper sulfate pentahydrate, 93.2 g/L mannitol, and 980 mg/L MES, pH 5.7) three times. After the washes, the samples are incubated on ice for 30 minutes and then washed 2 times with MMG (90.5 g/L mannitol, 1.428 g/L magnesium chloride, 780 mg/L MES, pH 5.7). After the final wash with MMG, 20 μL of protoplast sample is imaged under an EVOS microscope using DHC-F0l disposable hemocytometers to get an approximate cell count per sample. Each sample is diluted to about 800,000 cells per mL. Once diluted, the protoplasts are incubated with 10 uL of purified DNA from E. coli, containing various combinations of pMOZ plasmids and PEG solution (14.8 g/L calcium chloride, 26.6 g/L mannitol, and 400 g/L PEG 4000) to initiate PEG mediated gene transfer.

In one condition the protoplasts are transformed with purified DNA containing pMOZ401. In a second condition the protoplasts are transformed with purified DNA containing pMOZ702 and pMOZl1. In a third condition the protoplasts are transformed with purified DNA containing pMOZ882.

Following incubation with PEG solution and DNA containing pMOZ plasmids, the protoplasts are washed three times with CPW9M and incubated at 25° C. in the dark for 72 hours. After 72 hours, the samples are spun down at 16.1 RCF for 5 minutes. The supernatant is removed and 50 uL of protein extraction buffer (800 uL of 500 mM sodium phosphate, 200 uL of 500 mM sodium phosphate di basic, 1 mL 200 mM Sodium metabisulfite, 50 uL Tween-20, 5 mL IM Trehalose, 3 mL diH2O) is added. The samples go through 3 cycles of 2 minutes in liquid nitrogen followed by 3 minutes in a 37° C. water bath. After three freeze-thaw cycles are complete, the mixtures are incubated on a rotisserie at 4 C for 1 hour and then centrifuged at 4000RPM in with an Eppendorf 54 1 5R centrifuge to pellet the transformed protoplasts. The supernatant containing the extracted protein is transferred to a new 1.7 mL tube.

Further continuing this example, protein samples from transformed protoplasts are analyzed with sodium dodecyl sulfate polyacrylamide gel electrophoresis (SDS PAGE). 20 μL of supernatant is mixed with 6.6 uL of protein loading buffer (900 uL of 4× Laemmli Sample Buffer [Bio-Rad Laboratories]+lO 0 uL of 2-mercaptoethanol) and heated for 5 minutes at 95 C. These samples are loaded onto Bio-Rad “Any kD” precast polyacrylamide gels along with a standard protein ladder and phosphorylated and dephosphorylated beta casein samples from Sigma Aldrich. The gel is run in 1× Tris/Glycine/SOS Buffer (Bio-Rad Laboratories) at 150V for 45 minutes. The gel is removed from the gel box and placed in a PVDF Transfer Pack (Bio-Rad Laboratories), the transfer pack is placed in a Trans-Blot Turbo (Bio-Rad Laboratories) and the proteins are transferred to the PVDF membrane using the “Mini TGX” settings. The PVDF membrane containing the transferred proteins is first washed in 25 mL Protein Free Blocking Buffer (Thermo Fisher) for 1 hour, then incubated with 5 mL of Protein Free Blocking Buffer containing 5 μL of anti-beta-casein poly clonal rabbit IgG at room temperature for 4 hours. The membrane is then washed three times with 10 mL TBST (20 mM Tris-HCl, 150 mM NaCl, 0.1% Tween 20) for 10 minutes at room temperature. Then the membrane is washed with 25 mL of Protein Free Blocking Buffer containing 2 μL of anti-rabbit IgG secondary antibody conjugated to horseradish peroxidase for 1 hour at room temperature and then poured off. The membrane is placed in a ChemiDoc MP imaging system (Bio-Rad Laboratories) and 1 mL of SuperSignal West Pico (ThermoFisher) luminescent imaging solution is added to the membrane. Images are captured using the Chemiluminescence setting on the ChemiDoc MP.

Further continuing this example, the casein proteins expressed in plant cells are probed on the anti-beta-casein Western blot to measure varying migration distances. In lanes containing beta casein coexpressed with a human kinase, two human kinases, a bovine kinase, or two bovine kinases, the bands are shifted upward on the gel relative to the sample transformed with only beta casein, showing that the molecular weight of the beta casein has increased suggesting the casein proteins are phosphorylated by the kinase or kinases.

As used herein, a “vector” is a plasmid comprising operably linked polynucleotide sequences that facilitate expression of a coding sequence in a particular host organism (e.g., a bacterial expression vector or a plant expression vector). Polynucleotide sequences that facilitate expression in prokaryotes can include, e.g., a promoter, an enhancer, an operator, and a ribosome binding site, often along with other sequences. Eukaryotic cells can use promoters, enhancers, termination and polyadenylation signals and other sequences that are generally different from those used by prokaryotes.

A specified nucleic acid is “derived from” a given nucleic acid when it is constructed using the given nucleic acid's sequence, or when the specified nucleic acid is constructed using the given nucleic acid. For example, a cDNA or EST is derived from an expressed mRNA.

As used herein, the term “plant” includes whole plant, plant organ, plant tissues, and plant cell and progeny of same, but is not limited to angiospems and gymnosperms such as Arabidopsis, potato, tomato, tobacco, alfalfa, lemice, carrot, strawberry, sugarbeet, cassava, sweet potato, soybean, lima bean, pea, chick pea, maize (com), turf grass, wheat, rice, barley, sorghum, oat, oak, eucalyptus, walnut, palm and duckweed a well as fern and moss. Thus, a plant may be a monocot, a dicot, a vascular plant reproduced from spores such as fern or a nonvascular plant such as moss, liverwort, hornwort and algae. The term “plant,” as used herein, also encompasses plant cells, seeds, plant progeny, propagule whether generated sexually or asexually, and descendants of any of these, such as cuttings or seed. Plant cells include suspension cultures, callus, embryos, meristematic regions, callus tissue, leaves, roots, shoots, gametophytes, sporophytes, pollen, seeds and microspores. Plants may be at various stages of maturity and may be grown in liquid or solid culture, or in soil or suitable media in pots, greenhouses or fields.

As used herein, the term “dicot” refers to a flowering plant whose embryos have two seed leaves or cotyledons. Examples of dicots include Arabidopsis, tobacco, tomato, potato, sweet potato, cassava, alfalfa, lima bean, pea, chick pea, soybean, carrot, strawberry, lettuce, oak, maple, walnut, rose, mint, squash, daisy, quinoa, buckwheat, mung bean, cow pea, lentil, lupin, peanut, fava bean, French beans, mustard, or cactus.

As used herein, the term “monocot” refers to a flowering plant whose embryos have one cotyledon or seed leaf. Examples of monocots include turf grass, maize (corn), rice, oat, wheat, barley, sorghum, orchid, iris, lily, onion, palm, and duckweed.

As used herein, the term “transgenic plant” means a plant that has been transformed with one or more exogenous nucleic acids. “Transformation” refers to a process by which a nucleic acid is stably integrated into the genome of a plant cell. “Stably transformed” refers to the permanent, or non-transient, retention, expression, or a combination thereof of a polynucleotide in and by a cell genome. A stably integrated polynucleotide is one that is a fixture within a transformed cell genome and can be replicated and propagated through successive progeny of the cell or resultant transformed plant. Transformation can occur under natural or artificial conditions using various methods. Transformation can rely on any method for the insertion of nucleic acid sequences into a prokaryotic or eukaryotic host cell, including Agrobacterium-mediated transformation as illustrated in U.S. Pat. Nos. 5,159,135; 5,824,877; 5,591,616 and 6,384,301, all of which are incorporated herein by reference in its entirety.

Methods for plant transformation also include microprojectile bombardment as illustrated in U.S. Pat. Nos. 5,015,580; 5,550,318; 5,538,880; 6,153,812; 6,160,208; 6,288,312 and 6,399,861, all of which are incorporated herein by reference in its entirety. Recipient cells for the plant transformation include meristem cells, callus, immature embryos, hypocotyls explants, cotyledon explants, leaf explants, and gametic cells such as microspores, pollen, sperm and egg cells, and any cell from which a fertile plant can be regenerated, as described in U.S. Pat. Nos. 6,194,636; 6,232,526; 6,541,682 and 6,603,061 and U.S. Patent Application publication US 2004/0216189 A1, all of which are incorporated herein by reference in its entirety.

As used herein, the term “stably expressed” refers to expression and accumulation of a protein in a plant cell over time. As an example, a recombinant protein may accumulate because it is not degraded by endogenous plant proteases. As a further example, a recombinant protein is considered to be stably expressed in a plant if it is present in the plant in an amount of 1% or higher per total protein weight of soluble protein extractable from the plant.

As used herein, the term “recombinant” refers to nucleic acids or proteins formed by laboratory methods of genetic recombination (e.g., molecular cloning) to bring together genetic material from multiple sources, creating sequences that would otherwise not be found in the genome. Recombinant proteins may be expressed in vivo in various types of host cells, including plant cells, bacterial cells, fungal cells, avian cells, and mammalian cells. Recombinant proteins may also be generated in vitro. As used herein, the term “tagged protein” refers to a recombinant protein that includes additional peptides that are not part of the native protein and that remain after post-translational processing.

These and other valuable aspects of the embodiments of the present disclosure consequently further the state of the technology to at least the next level. While the disclosure has been described in conjunction with a specific best mode, it is to be understood that many alternatives, modifications, and variations will be apparent to those skilled in the art in light of the descriptions herein. Accordingly, it is intended to embrace all such alternatives, modifications, and variations that fall within the scope of the included claims. All matters set forth herein or shown in the accompanying drawings are to be interpreted in an illustrative and non-limiting sense.

As used herein, the phrases “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

As used herein, the singular forms “a”, “an” and “the” are intended to include the plural forms as well, unless the context clearly indicates otherwise. Furthermore, to the extent that the terms “including”, “includes”, “having”, “has”, “with”, or variants thereof are used in either the detailed description and/or the claims, such terms are intended to be inclusive in a manner similar to the term “comprising.”

Use of absolute or sequential terms, for example, “will,” “will not,” “shall,” “shall not,” “must,” “must not,” “first,” “initially,” “next,” “subsequently,” “before,” “after,” “lastly,” and “finally,” are not meant to limit scope of the present embodiments disclosed herein but as exemplary.

While some instances of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

These and other valuable aspects of the embodiments of the present disclosure consequently further the state of the technology to at least the next level. While the disclosure has been described in conjunction with a specific best mode, it is to be understood that many alternatives, modifications, and variations will be apparent to those skilled in the art in light of the descriptions herein. Accordingly, it is intended to embrace all such alternatives, modifications, and variations that fall within the scope of the included claims. All matters set forth herein or shown in the accompanying drawings are to be interpreted in an illustrative and non-limiting sense.

1. An expression system for phosphorylating heterologous proteins comprising:
a plant cell comprising a transfected bacterium inserted into the plant cell, wherein the transfected bacterium comprises polynucleotides for expressing the heterologous protein selected from:
one or more heterologous ruminant casein proteins; and
a non-plant heterologous kinase;
wherein the plant cell is an Arabidopsis thaliana cell;
wherein the transfected bacterium is E. coli or Agrobacterium.

2. The expression system of claim 1, wherein the Arabidopsis thaliana cell comprises:
the one or more heterologous ruminant casein proteins selected from the group consisting of k-casein, αS1-casein, αS2-casein, and β-casein; and
the heterologous non-plant kinase;
wherein both proteins are expressed in the plant cell;
wherein the heterologous non-plant kinase phosphorylates the heterologous ruminant casein protein in vivo.

3. The expression system of claim 1, wherein αS1 casein is most abundant, followed by β casein as second next most abundant, αS2-casein as second least abundant, and κ casein as the least abundant heterologous ruminant casein proteins expressed in the Arabidopsis thaliana cell.

4. The expression system of claim 1, wherein the polynucleotides regulate cytoplasmic concentrations of minerals.

5. The expression system of claim 4, wherein the regulated cytoplasmic concentration of the minerals enhances formation of the one or more heterologous ruminant casein proteins.

6. The expression system of claim 1, wherein the one or more heterologous ruminant casein proteins are localized to endoplasmic reticulum.

7. The expression system of claim 1, wherein the heterologous non-plant kinase is a human kinase.

8. The expression system of claim 1, wherein the heterologous non-plant kinase is a ruminant kinase.

9. A method for modifying a plant cell comprising:
introducing nucleic acids into the plant cell, wherein the nucleic acids encode for one or more heterologous ruminant casein proteins; and a non-plant heterologous kinase; and
expressing the one or more heterologous ruminant casein proteins and the non-plant heterologous kinase in the plant cell, thereby phosphorylating the one or more heterologous ruminant casein proteins with the non-plant heterologous kinase in vivo;
wherein the plant cell is wherein the plant cell is an Arabidopsis thaliana cell.

10. The method of claim 9, wherein the one or more heterologous ruminant casein proteins is selected from the group consisting of k-casein, αS1-casein, αS2-casein, and β-casein.

11. The method of claim 9, wherein introducing the nucleic acids into the plant cell comprises: regulating cytoplasmic concentrations of minerals.

12. The method of claim 9, wherein expressing the one or more heterologous ruminant casein proteins and the non-plant heterologous kinase in the plant cell comprises: localizing the one or more heterologous ruminant casein proteins and the non-plant heterologous kinase to a vacuole of the plant cell.

13. The method of claim 11, wherein regulating the cytoplasmic concentration of the minerals enhances formation of the one or more heterologous ruminant casein proteins.

14. The method of claim 9, phosphorylating the one or more heterologous ruminant casein proteins with the non-plant heterologous kinase in vivo increases aggregation of the one or more heterologous ruminant casein proteins with each other.

15. The method of claim 9, phosphorylating the one or more heterologous ruminant casein proteins with the non-plant heterologous kinase in vivo increases viscosity of a solution containing the phosphorylated one or more heterologous ruminant casein proteins.

16. The method of claim 9, phosphorylating the one or more heterologous ruminant casein proteins with the non-plant heterologous kinase in vivo increases calcium binding.

17. An isolated phosphorylated heterologous ruminant casein protein produced in an Arabidopsis thaliana cell.

18. The isolated phosphorylated heterologous ruminant casein protein of claim 17 comprises calcium and phosphate.

19. The isolated phosphorylated heterologous ruminant casein protein of claim 17 comprises a phosphorylation level substantially identical to a phosphorylation level of β-casein from milk.

20. The isolated phosphorylated heterologous ruminant casein protein of claim 17 comprises a phosphorylation level substantially identical to a phosphorylation level of k-casein from milk.
